SEQUENCE LISTING (11 sequences)

SEQ ID NO: 1 - protein, 297 aa, Homo sapiens
GNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFLALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTN

SEQ ID NO: 2 - protein, 320 aa, Homo sapiens
GHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVE

SEQ ID NO: 3 - protein, 309 aa, Homo sapiens
GNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKRPPGLEYSYNPSHNPEEQLSSRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRR

SEQ ID NO: 4 - protein, 293 aa, Homo sapiens
GAMDPSSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVSAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKRGT

SEQ ID NO: 5 - protein, 268 aa, Homo sapiens
PNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAPAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMF

SEQ ID NO: 6 - protein, 272 aa, Homo sapiens
EYDLKWEFPRENLEFGKVLGSGAFGKVMNATAYGISKTGVSIQVAVKMLKEREALMSELKMMTQLGSHENIVNLLGACTLSGPIYLIFEYCCYGDLLNYLRSKREKFLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQL

SEQ ID NO: 7 - protein, 264 aa, Homo sapiens
DDWEIPDGQITVGQRIGSGSFGTVYKGKWHGDVAVKMLNVTAPTPQQLQAFKNEVGVLRKTRHVNILLFMGYSTAPQLAIVTQWCEGSSLYHHLHIIETKFEMIKLIDIARQTAQGMDYLHAKSIIHRDLKSNNIFLHEDLTVKIGDFGLATVLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPK

SEQ ID NO: 8 - protein, 5 aa, Homo sapiens
NCIHR

SEQ ID NO: 9 - protein, 8 aa, Homo sapiens
ICDFGLAR

SEQ ID NO: 10 - protein, 4 aa, Artificial Sequence
  Description of Artificial Sequence Synthetic peptide
GSGS

SEQ ID NO: 11 - protein, 298 aa, Homo sapiens
HKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKTSPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHI

(19) United States
(12) Patent Application Publication
(10) Pub. No.: US 2010/0185419 A1
(43) Pub. Date: Jul. 22, 2010

(54) ALGORITHM FOR DESIGNING IRREVERSIBLE INHIBITORS

(75) Inventors: Juswinder Singh, Ashland, MA (US); Russell Colyn Petter, Stow, MA (US); Dequiang Niu, Lexington, MA (US)

Correspondence Address: McDermott Will & Emery, 600 13th Street, NW, Washington, DC, 20005-3096, US

(73) Assignee: Avila Therapeutics, Inc., Waltham, MA (US)

(21) Appl. No.: 12/554,433

(22) Filed: Sep. 4, 2009

Related U.S. Application Data
(60) Provisional application No. 61/094,782, filed on Sep. 5, 2008.

(51) Int. Cl. G06F 17/50; C07D 209/52; C07D 215/12; C07K 14/00

(52) U.S. Cl. 703/1; 548/515; 546/173; 530/300; 703/2

(57) ABSTRACT

The invention is an algorithm and method for designing an inhibitor that covalently binds a target polypeptide. The algorithm and method can be used to rapidly and efficiently convert reversible inhibitors into irreversible inhibitors.

### BACKGROUND OF THE INVENTION

Compounds that inhibit the activity of polypeptides, such as enzymes, are important therapeutic agents. Most inhibitors reversibly bind to their target polypeptides and reversibly inhibit the activity of their target polypeptides.

Although reversible inhibitors have been developed that are efficacious therapeutic agents, reversible inhibitors have certain disadvantages. For example, many reversible inhibitors of kinases interact with the ATP-binding site. Because the structure of the ATP-binding site is highly conserved among kinases, it has been very challenging to develop reversible inhibitors that selectively inhibit one or more desired kinases. In addition, because reversible inhibitors dissociate from their target polypeptides, the duration of inhibition may be shorter than desired. Thus, when reversible inhibitors are used as therapeutic agents higher quantities and/or more frequent dosing than is desired may be required in order to achieve the intended biological effect. This may produce toxicity or result in other undesirable effects.

Irreversible inhibitors that covalently bind to their target polypeptides have been described. Covalent irreversible inhibitors of drug targets have a number of important advantages over their reversible counterparts as therapeutics. Prolonged suppression of the drug targets may be necessary for maximum pharmacodynamic effect and an irreversible inhibitor can provide this advantage by permanently eliminating existing drug target activity, which will return only when new target polypeptide is synthesized. When an irreversible inhibitor is administered, the therapeutic plasma concentration of the irreversible inhibitor would need to be attained only long enough to briefly expose the target polypeptides to the inhibitor, which would irreversibly suppress activity of the target. Plasma levels could then rapidly decline while the target polypeptide would remain inactivated. This has the potential advantage of lowering the minimal plasma concentration at which therapeutic activity occurs, minimizing multiple dosing requirements and eliminating the requirement for long plasma half-lives without compromising efficacy. All of these considerations could reduce toxicity due to any nonspecific off target interactions that may occur at high or prolonged plasma levels. Irreversible inhibitors would also likely have advantages in overcoming drug resistance.

US 2007/0082884 describes the use of structural bioinformatics to identify a Cys in a binding site of several kinases that is available for modification by a small molecule inhibitor. The document also describes the preparation of compounds that form a covalent bond with the identified Cys. Pan et al. ChemMedChem 2(1):58-61 (2007), describe the identification of a scaffold capable of inhibiting Bruton's tyrosine kinase (BTK) from a screening campaign, the preparation of a series of compounds based on the scaffold, and the identification of covalent inhibitors of BTK. Wissner et al., J. Med. Chem. 48(24):7560-81 (2005), describe the preparation of a series of compounds that are covalent irreversible inhibitors of vascular endothelial growth factor receptor-2 (VEGFR2) kinase. The compounds contain a quinazoline core structure and a highly reactive quinone. None of these references describe a generalizable method for designing irreversible inhibitors, or for designing an irreversible analog of a known reversible inhibitor. Such a method would substantially reduce the time and cost of developing irreversible inhibitors.

### SUMMARY OF THE INVENTION

The invention relates to an algorithm and method for designing irreversible inhibitors of a target polypeptide. The irreversible inhibitors designed by the algorithm and methods described herein form a covalent bond with an amino acid side chain in the target polypeptide. Now, using the invention, it is possible to efficiently design an irreversible inhibitor starting from a known reversible inhibitor. This approach reduces the time and costs associated with traditional screening and structure activity relationship development approaches to drug discovery and development. The algorithm and method include forming a bond between the candidate irreversible inhibitor and the target polypeptide.

The algorithm and method comprises A) providing a structural model of a reversible inhibitor bound to a binding site in a target polypeptide, wherein the reversible inhibitor makes non-covalent contacts with the binding site; B) identifying a Cys residue in the binding site of the target polypeptide that is adjacent to the reversible inhibitor when the reversible inhibitor is bound to the binding site; C) producing structural models of candidate inhibitors that covalently bind the target polypeptide, wherein each candidate inhibitor contains a warhead that is bonded to a substitutable position of the reversible inhibitor, the warhead comprising a reactive chemical functionality and optionally a linker that positions the reactive chemical functionality within bonding distance of the Cys residue in the binding site of the target polypeptide; D) determining the substitutable positions of the reversible inhibitor that result in the reactive chemical functionality of the warhead being within bonding distance of the Cys residue in the binding site of the target polypeptide when the candidate inhibitor is bound to the binding site; E) for a candidate inhibitor that contains a warhead that is within bonding distance of the Cys residue in the binding site of the target polypeptide when the candidate inhibitor is bound to the binding site, forming a covalent bond between the sulfur atom of the Cys residue in the binding site and the reactive chemical functionality of the warhead when the candidate inhibitor is bound to the binding site. A covalent bond length of less than about 2 angstroms for the bond formed between the sulfur atom of the Cys residue in the binding site and the reactive chemical functionality of the warhead, indicates that the candidate inhibitor is an inhibitor that will covalently bind a target polypeptide.

### BRIEF DESCRIPTION OF THE DRAWINGS

FIG. 1A-1Q illustrates the structures of 114 exemplary warheads that can be used in the invention, and the thiol adducts that each warhead forms with a Cys residue in a target polypeptide. In the thiol adducts, the sulfur atom of the Cys side chain is bonded to the warhead and to the 13 carbon of the Cys reside, and the 13 carbon of the Cys reside is bonded to R. R represents the remainder of the target polypeptide.

FIG. 2A is an image of a model of Compound 1 in the ATP-binding site of c-KIT. The target Cys residue, Cys788 of c-KIT, also shown.

FIG. 2B is an image of a model of Compound 1 in the ATP-binding site of c-KIT. In this image, Compound 1 has formed a covalent bond with Cys788 of c-KIT.

FIG. 3A is an image of a model of Compound 4 in the ATP-binding site of FLT3. The target Cys residue, Cys828 of FLT3, is also shown.

FIG. 3B is an image of a model of Compound 4 in the ATP-binding site of FLT3. In this image, Compound 4 has formed a covalent bond with Cys828 of FLT3.

FIG. 4A is an image of a model of Compound 5 in the binding site of Hepatitis C Virus (HCV) protease, more specifically the NS3/4A HCV protease component of the virus. The target Cys residue, Cys159 of HCV protease, is also shown.

FIG. 4B is an image of a model of Compound 5 in the binding site of HCV protease. In this image, Compound 5 has formed a covalent bond with Cys159 of HCV protease.

FIG. 5 depicts the dose response inhibition of cell proliferation of EOL-1 cells with reference compound and Compound 2.

FIG. 6 depicts the inhibition of PDGFR with reference compound and Compound 2 in a “washout” experiment using EOL-1 cells.

FIG. 7 depicts the results of mass spectral analysis of a tryptic digest of PDGFR that was treated with Compound 3. The results confirm that Compound 3 formed a bond with Cys814.

FIG. 8 depicts the results of mass spectral analysis of NS3/4A HCV protease that was treated with Compound 5. The results show that Compound 5-treated HCV protease increased in mass, consistent with the formation of an adduct between the protein and Compound 5. The adduct was not formed with a mutant form of HCV protease in which Cys 159 was replaced with Ser.

FIG. 9 depicts the results of mass spectral analysis of HCV NS3/4A protease that was treated with Compound 6. The results show that Compound 6-treated HCV protease increased in mass, consistant with the formation of an adduct between the protein and Compound 6. The adduct was not formed with a mutant form of HCV protease in which Cys159 was replaced with Ser.

FIGS. 10A and 10B are histograms showing prolonged inhibition of cKIT activity by the irreversible inhibitor Compound 7 relative to sorafenib in a cKIT phosphorylation assay (10A) and downstream signaling assay that measured ERK phosphorylation (10B).

FIG. 11 depicts the results of mass spectral analysis of HCV NS3/4A protease that was treated with Compound 8. The results show that Compound 8-treated HCV protease increased in mass, consistent with the formation of an adduct between the protein and Compound 8.

### DETAILED DESCRIPTION OF THE INVENTION

### Definitions

As used herein, “adjacent” refers to an amino acid residue in a target polypeptide that is near a reversible inhibitor when the reversible inhibitor is bound to the target polypeptide. For example, an amino acid residue in a target polypeptide is adjacent to a reversible inhibitor when any non-hydrogen atom of the amino acid residue is within about 20A, about 18A, about 16A, about 14A, about 12A, about 10A, about 8A, about 6A, about 4A, or about 2A, of any non-hydrogen atom of a reversible inhibitor when the reversible inhibitor is bound to the target polypeptide. An amino acid residue in a target polypeptide that contacts a reversible inhibitor when the reversible inhibitor is bonded to the target polypeptide is adjacent to the reversible inhibitor.

As used herein, “substitutable position” refers to non-hydrogen atoms in a reversible inhibitor that are bonded to other atoms or chemical groups (e.g., Hydrogen) that can be replaced and/or removed without affecting binding of the reversible inhibitor to the target polypeptide.

As used herein, binding of a reversible inhibitor is “not affected” when the binding mode and residence time of the reversible inhibitor in the target binding site is substantially unchanged. Binding of a reversible inhibitor is not affected, for example, when the potency of the inhibitor in a suitable assay (e.g., IC50, Ki) is changed by less than a factor of 1000, less than a factor of 100 or less than a factor of 10.

As used herein, “bonding distance” refers to a distance of not more than about 6A, not more than about 4A, or not more than about 2A.

As used herein, “covalent bond” and “valence bond” refer to a chemical bond between two atoms created by the sharing of electrons, usually in pairs, by the bonded atoms.

As used herein, “non-covalent bond” refers to an interaction between atoms and/or molecules that does not involve the formation of a covalent bond between them.

As used herein, an “irreversible inhibitor” is a compound that covalently binds a target polypeptide through a substantially permanent covalent bond and inhibits the activity of the target polypeptide for a period of time that is longer than the functional life of the protein. Irreversible inhibitors usually are characterized by time dependency, i.e., the degree of inhibition of the target polypeptide increases, until activity is eradicated, with the time that the target polypeptide is in contact with the irreversible inhibitor. Recovery of target polypeptide activity when inhibited by an irreversible inhibitor is dependent upon new protein synthesis. Target polypeptide activity that is inhibited by an irreversible inhibitor remains substantially inhibited in a “wash out” study. Suitable methods for determining if a compound is an irreversible inhibitor are well-known in the art. For example, irreversible inhibition can be identified or confirmed using kinetic analysis (e.g., competitive, uncompetitive, non-competitive) of the inhibition profile of the compound with the target polypeptide, the use of mass spectrometry of the protein drug target modified in the presence of the inhibitor compound, discontinuous exposure, also known as “washout” studies, and the use of labeling, such as radiolabelled inhibitor, to show covalent modification of the enzyme, or other methods known to one of skill in the art. In certain preferred embodiments, the target polypeptide has catalytic activity and the irreversible inhibitor forms a covalent bond with a Cys reside that is not a catalytic residue.

As used herein, a “reversible inhibitor” is a compound that reversibly binds a target polypeptide and inhibits the activity of the target polypeptide. A reversible inhibitor may bind its target polypeptide non-covalently or through a mechanism that includes a transient covalent bond. Recovery of target polypeptide activity when inhibited by a reversible inhibitor can occur by dissociation of the reversible inhibitor from the target polypeptide. Target polypeptide activity is recovered when a reversible inhibitor is “washed out” in a wash out study. Preferred reversible inhibitors are “potent” inhibitors of the activity of their target polypeptides. A “potent” reversible inhibitor inhibits the activity of its target polypeptide with an IC₅₀ of about 50 μM or less, about 1 μM or less, about 100 nM, or less, or about 1 nM or less, and/or a Kᵢ of about 50 μM or less, about 1 μM or less, about 100 nM, or less, or about 1 nM or less.

The terms “IC₅₀” and “inhibitory concentration 50” are terms of art that are well-understood to mean the concentration of a molecule that inhibits 50% of the activity of a biological process of interest, including, without limitation, catalytic activity, cell viability, protein translation activity and the like.

The term “Kᵢ” and “inhibition constant” are terms of art that are well-understood to be the dissociation constant for the polypeptide (e.g., enzyme)-inhibitor complex.

As used herein, a “substantially permanent covalent bond” is a covalent bond between an inhibitor and the target polypeptide that persists under physiological conditions for a period of time that is longer than the functional life of the target polypeptide.

As used herein, a “transient covalent bond” is a covalent bond between an inhibitor and the target polypeptide that persists under physiological conditions for a period of time that is shorter than the functional life of the target polypeptide.

As used herein, a “warhead” is a chemical group comprising a reactive chemical functionality or functional group and optionally containing a linker moiety. The reactive functional group can form a covalent bond with an amino acid residue such as cysteine (i.e., the —SH group in the cysteine side chain), or other amino acid residue capable of being covalently modified that is present in the binding pocket of the target protein, thereby irreversibly inhibiting the target polypeptide. It will be appreciated that the -L-Y group, as defined and described herein below, provides such warhead groups for covalently, and irreversibly, inhibiting the protein.

The term “in silico” is a term of art that is understood to refer to methods and processes that are performed on a computer, for example, using computational modeling programs, computational chemistry, molecular graphics, molecular modeling, and the like to produce computer simulations.

As used herein, the term “computational modeling programs” refers to computer software programs that deal with the visualization and engineering of proteins and small molecules, including but not limited to computational chemistry, chemoinformatics, energy calculations, protein modeling, and the like. Examples of such programs are known to one of ordinary skill in the art, and certain examples are provided herein.

As used herein, the term “sequence alignment” refers to an arrangement of two or more protein or nucleic acid sequences, which allows comparison and highlighting of their similarity (or difference). Methods and computer programs for sequence alignment are well known (e.g., BLAST). Sequences may be padded with gaps (usually denoted by dashes) so that wherever possible, columns contain identical or similar characters from the sequences involved.

As used herein, the term “crystal” refers to any three-dimensional ordered array of molecules that diffracts X-rays.

As used herein, the terms “atomic co-ordinates” and “structure co-ordinates” refers to mathematical co-ordinates (represented as “X,” “Y” and “Z” values) that describe the positions of atoms in a three-dimensional model/structure or experimental structure of a protein.

As used herein, the term “homology modeling” refers to the practice of deriving models for three-dimensional structures of macromolecules from existing three-dimensional structures for their homologues. Homology models are obtained using computer programs that make it possible to alter the identity of residues at positions where the sequence of the molecule of interest is not the same as that of the molecule of known structure.

As used herein, “computational chemistry” refers to calculations of the physical and chemical properties of molecules.

As used herein, “molecular graphics” refers to two or three dimensional representations of atoms, preferably on a computer screen.

As used herein, “molecular modeling” refers to methods or procedures that can be performed with or without a computer to make one or more models, and, optionally, to make predictions about structure activity relationships of ligands. The methods used in molecular modeling range from molecular graphics to computational chemistry.

The invention relates to algorithms and methods for designing irreversible inhibitors of target polypeptides, such as enzymes. The irreversible inhibitors designed using the invention are capable of potent and selective inhibition of the target polypeptide. In general, the invention is a rational algorithm and design method in which design choices are guided by the structure of the target polypeptide, the structure of a reversible inhibitor of the target polypeptide, and the interaction of the reversible inhibitor with the target polypeptide. Irreversible inhibitors, or candidate irreversible inhibitors, designed using the method of the invention comprise a template or scaffold to which one or more warheads are bonded. The resulting compound has binding affinity for the target polypeptide and once bound, the warhead reacts with a Cys residue in the binding site of the target polypeptide to form a covalent bond, resulting in irreversible inhibition of the target polypeptide.

The invention provides a method for designing an inhibitor that covalently binds a target polypeptide. The method includes providing a structural model of a reversible inhibitor bound to a binding site in a target polypeptide. The reversible inhibitor makes non-covalent contacts with the binding site. Using the structural model, a Cys residue in the binding site of the target polypeptide that is adjacent to the reversible inhibitor when the reversible inhibitor is bound to the binding site is identified. A single Cys residue, all Cys residues or a desired number of Cys residues that are adjacent to the reversible inhibitor when the reversible inhibitor is bound to the binding site can be identified.

Structural models of one or more candidate inhibitors that are designed to covalently bind the target polypeptide are produced. The candidate inhibitors contain a warhead that is bonded to a substitutable position of the reversible inhibitor. The warhead contains a reactive chemical functionality capable or reacting with and forming a covalent bond with the thiol group in the side chain of a Cys reside, and optionally a linker that positions the reactive chemical functionality within bonding distance of one of the identified Cys residue in the binding site of the target polypeptide. Substitutable positions of the reversible inhibitor that result in the reactive chemical functionality of the warhead being within bonding distance of an identified Cys residue in the binding site of the target polypeptide when the candidate inhibitor is bound to the binding site are identified.

A determination of whether a candidate irreversible inhibitor containing a warhead that is attached to an identified substitutable position and is within bonding distance of an identified Cys residue in the binding site of the target polypeptide when the candidate inhibitor is bound to the binding site is likely to be an inhibitor that covalently binds the target polypeptide, and preferably is an irreversible inhibitor of the target polypeptide, is made by forming a covalent bond between the sulfur atom of the Cys residue in the binding site and the reactive chemical functionality of the warhead when the candidate inhibitor is bound to the binding site. A covalent bond length of about 2.1 angstroms to about 1.5 angstroms, or of less than about 2 angstroms, for the bond formed between the sulfur atom of the Cys residue in the binding site and the reactive chemical functionality of the warhead, indicates that the candidate inhibitor is an inhibitor that covalently binds a target polypeptide.

The method of the invention can be performed using any suitable structural model, such as physical models or preferably molecular graphics. The method can be performed manually or can be automated. Preferably, the method is performed in silico.

As will be apparent from the foregoing and more detailed description that follows, conceptually the algorithm and method of the invention comprises A) providing a target and reversible inhibitor, B) identifying a target Cysteine, C) producing structural models of candidate inhibitors that contain a warhead, D) determining proximity of warhead to target Cysteine, and E) forming a covalent bond.

### A) Provide a Target and Reversible Inhibitor

The invention comprises providing a structural model of a reversible inhibitor bound to a binding site in a target polypeptide, in which the reversible inhibitor makes non-covalent contacts with the binding site. Any suitable structural model of a reversible inhibitor bound to a binding site in a target polypeptide can be provided and used. Generally, a known or pre-existing potent reversible inhibitor of a target polypeptide is used to provide a starting point (e.g., a template or scaffold) for designing an inhibitor that covalently binds a target polypeptide using the invention. Thus, for example, when a reversible inhibitor of a target protein has previously been identified (e.g., reported in the literature or identified by any method known to one of ordinary skill in the art), the known reversible inhibitor can be used to generate a structural model of the target polypeptide complexed with the inhibitor. However, if desired, a new or previously unknown reversible inhibitor can be used to generate a structural model of the target polypeptide complexed with the inhibitor.

The algorithm and method can be used to design irreversible inhibitors using any suitable reversible inhibitor, such as a potent reversible inhibitor, a weak reversible inhibitor or a reversible inhibitor of moderate potency. For example, as described in Example 8 the algorithm and method of the invention can be used to increase potency of reversible inhibitors by designing in the capability to covalently bind to the target protein. In some embodiments, the algorithm and method employs the structure of a potent reversible inhibitor. In other embodiments, the algorithm and method are used to improve potency by designing in covalent binding, and employs the structure of an inhibitor of weak or moderate potency, such as an inhibitor with an IC₅₀ or Kᵢ that is ≧10 nM, ≧100 nM, between about 1 μM and about 10 nM, between about 1 μM and about 100 nM, between about 100 μM and 1 μM, or between about 1 mM and about 1 μM.

The three-dimensional structure of many suitable target polypeptides are known and readily available from public sources, such as the Research Collaboratory for Structural Bioinformatics Protein Data Bank (RCSB PDB available on line at world wide web pdb.org; see also H. M. Berman et al.,: Nucleic Acids Research, 28 pp. 235-242 (2000) and www.rcsb.org), and worldwide Protein Data Bank (wwPDB; Berman et al, Nature Structural Biology 10(12):980 (2003)). A nonlimiting list of suitable target polypeptides for which structures are available in the Protein Data Bank is presented in Table 1. If desired, the three-dimensional structure of a target protein can be obtained using any suitable method. Suitable methods for determining structure are well-known and conventional in the art, such as solution-phase nuclear magnetic resonance (NMR) spectroscopy, solid-phase NMR spectroscopy, X-ray crystallography, and the like. (See, e.g., Blow, D, Outline of Crystallography for Biologists. Oxford: Oxford University Press. ISBN 0-19-851051-9 (2002).)

Structural models of target polypeptides can also be generated using well-known and conventional methods of computer modeling, such as homology modeling, or folding studies, based on, e.g., the primary and secondary structure of the protein. Suitable methods for producing homology models are well-known in the art. (See, e.g., John, B. and Sali, A. Nucleic Acids Res 31(14):3982-92 (2003).) Suitable programs for homology modeling include, for example, Modeler (Accelrys, Inc. San Diego) and Prime (Schrodinger Inc., New York). For example, as described herein in Example 3, a homology model of FLT3 kinase was produced based upon the known structure of Aurora kinase. A nonlimiting list of suitable target polypeptides for which sequence information is available that can be used to produce homology models is presented in Table 2. Preferred structural models are produced using the atomic coordinates for the target polypeptide, or at least the binding site of the target polypeptide, in complex with the reversible inhibitor. These atomic co-ordinates are available in the Protein Data Bank for many target polypeptides complexed with reversible inhibitors, and can be determined using X-ray crystallography, nuclear magnetic resonance spectroscopy, using homology modeling and the like.

Similarly, structural models of reversible inhibitors alone or complexed to a target polypeptide, can be generated based on known atomic coordinates or using other suitable methods. Suitable methods and programs for docking inhibitors onto target proteins are well-known in the art. (See, e.g., Perola et al., Proteins: Structure, Function, and Bioinformatics 56:235-249 (2004).) Generally, if the structure of a reversible inhibitor complexed to a target polypeptide is not known, a model of the complex can be prepared based on the possible or probable binding mode of the reversible inhibitor. Possible or probable binding modes for reversible inhibitors can be easily identified by a person of ordinary skill in the art, for example, based on structural similarity of the reversible inhibitor to another inhibitor with a known binding mode. For example, as described in Example 5, the structures of the complexes of HCV protease with more then 10 different inhibitors are known, and reveal that the inhibitors all have structural similarities in their binding modes to the protease. Based on this knowledge of the probable binding-mode of the reversible inhibitor V-1, a structural model of V-1 complexed to HCV protease was produced and used to successfully design an irreversible inhibitor that covalently bound Cys159 of HCV protease.

The structural model of a reversible inhibitor bound to a binding site in a target polypeptide is preferably a computer model. Computer models can be produced and visualized using any suitable software, such as, VIDA™, visualization software, (OpenEye Scientific Software, New Mexico), Insight II® or Discovery Studio®, graphic molecular modeling software (Accelrys Software Inc., San Diego, Calif.).

TABLE 1
Exemplary target polypeptides with structures in Protein
Data Bank

| | |
|---|---|
| Target Polypeptide | PDB CODE |

PROTEASE

| | |
|---|---|
| HUMAN CYTOMEGALOVIRUS | 2WPO |
| PROTEASE | |
| HEPATITIS C VIRUS NS3/4A | 2OC8 |
| PROTEASE | |
| HERPES PROTEASE | 1AT3 |
| KSHV PROTEASE | 1FL1 |
| VARICELLA-ZOSTER VIRUS | 1VZV |
| PROTEASE | |
| EPSTEIN BAR VIRUS PROTEASE | 1O6E |
| PROTEASOME | 1JD2 |
| CASPASE-1 | 1RWK |

PROTEIN KINASES

| | |
|---|---|
| MEK | 1S9J |
| BTK | 1K2P |
| CKIT | 1T46 |
| FLT3 | 1RJB |
| VEGFR2 | 1YWN |
| ZAP70 | 1U59 |
| C-SRC | 2SRC |
| JAK3 | 1YVJ |
| FAK | 1MP8 |
| EGFR | 1M17 |
| FGFR | 2FG1 |
| GSK3B | 1R0E |
| JNK3 | 1PMQ |

LIPID KINASES

| | |
|---|---|
| PI-3 kinase | 2CHW |

PHOSPHODIESTERASES

| | |
|---|---|
| PDE5 | 1TBF |

DEACETYLASES

| | |
|---|---|
| HDAC8 | 1VKG |

HEAT SHOCK PROTEINS

| | |
|---|---|
| HSP70 | 1S3X |

G PROTEIN-COUPLED RECEPTORS

| | |
|---|---|
| BETA ADRENERGIC RECEPTOR | 2VT4 |

TRANSFERASES

| | |
|---|---|
| FARNESYL TRANSFERASE | 1JCQ |

METALLOENZYMES

| | |
|---|---|
| CARBONIC ANHYDRASE | 1A42 |

NUCLEAR HORMONE RECEPTORS

| | |
|---|---|
| ANDROGEN RECEPTOR | 2AMB |
| ESTROGEN RECEPTOR ALPHA | 1R5K |
| PPAR DELTA | 1Y0S |

TABLE 2
Exemplary target polypeptides and sequence accession numbers

| Target Polypeptide | Sequence Accession Number |
|---|---|
| ZAP70 | P43403 |
| C-SRC | P12931 |
| ITK | NM_005546 |
| BTK | SWS: BTK_HUMAN |
| RON | Q04912 |
| JNK3 | SWS: MK10_HUMAN |
| FGFR1 | NM_000604 |
| FGFR2 | NM_000141 |
| FGFR3 | M58051 |
| FGFR4 | L03840 |
| KDR(FLK1, VEGFR, | NM_002253 |
| VEGFR2) | |
| FLT1 | AF063657 |
| FLT3 | NM_004119 |
| C-KIT | X06182 |
| EGFR | SWS: EGRF_HUMAN |
| PDFGR-A | M21574 |
| PDGFR-B | J03278 |
| GSK3A | NM_019884 |
| GSK3B | P49841 |
| RON | NM_002447 |
| C-YES | M15990 |
| Iuka | AF012890 |
| Icky | AF080158 |
| ALK | NM_004304 |
| JAK1 | P23458 |
| JAK2 | NM_004972 |
| JAK3 | AF513860 |
| TYK2 | P29597 |
| JNK1 | L26318 |
| JNK3 | P45984 |
| FAK | Q05397 |
| LIMK | NM_002314 |
| MEK1 | Q02750 |
| MEK2 | P36507 |
| MELK | NM_014791 |
| PBK | Q96KB5 |
| PDK2 | NM_016457 |
| PKR | P19525 |
| PLK | NM_004073 |
| PI3-KINASE | Q9BZC8 |
| hepatitis C virus | SWS: POLG_HCVJA |
| NS3/4A protease | |
| herpes simplex virus | GB: AAA92139 |
| protease | |
| KSHV protease | GB: AAC05223 |
| Varicella-Zoster virus | SWS: VP40_VZVD |
| protease | |
| Epstein-bar virus protease | SWS: VP40_EBV |
| human cytomegalovirus | SWS: VP40_HCMVA |
| protease | |

### B) Identify Target Cysteine

The invention comprises identifying a Cys residue in the binding site of the target polypeptide that is adjacent to the reversible inhibitor when the reversible inhibitor is bound to the binding site. Using the structural model of the target polypeptide complexed with the reversible inhibitor, Cys residues of the target polypeptide that are suitable for targeting for covalent bond formation with a warhead are identified. Cys residues that are suitable for targeting for covalent bond formation with a warhead are adjacent to the reversible inhibitor in the structural model. Cys residues that are adjacent to the reversible inhibitor in the structural model can be identified using any suitable method of determining intermolecular distances. Several programs for computing intermolecular distances in computer models are well-known in the art, such as, VIDA™, visualization software, (OpenEye Scientific Software, New Mexico), Discovery Studio, visualization software (Accelrys, Inc. San Diego), and the like.

In one example, the intermolecular distance (e.g., in angstroms) is determined between all non-hydrogen atoms of all Cys residues in the target polypeptide binding site and all non-hydrogen atoms of the reversible inhibitor. Cys residues that are adjacent to the reversible inhibitor are readily identified from these intermolecular distances. It is generally preferred that the adjacent Cys residue is within about 10 angstroms, about 8 angstroms, or about 6 angstroms of the reversible inhibitor.

If desired, Cys residues that are adjacent to the reversible inhibitor can be identified by analyzing changes in the accessible surface of the Cys residues in the target polypeptide. This can be achieved, for example, by determining the accessible surface area of the Cys residues in the target polypeptide (e.g., the inhibitor binding site of the target polypeptide) when the target polypeptide is complexed with the reversible inhibitor, and when the target polypeptide is not complexed with the reversible inhibitor. Cys residues that have a change in the accessible surface area when the reversible inhibitor is complexed to the target polypeptide are likely to be adjacent to the reversible inhibitor. See, e.g., Lee, B. and Richared, F. M., J. Mol. Biol. 55:379-400 (1971) regarding surface accessibility. This can be confirmed by determining intermolecular distances if desired.

C) Produce Structural Models of Candidate Inhibitors that Contain a Warhead

The invention comprises producing structural models of candidate inhibitors that are designed to covalently bind the target polypeptide, wherein each candidate inhibitor contains a warhead that is bonded to a substitutable position of the reversible inhibitor. Candidate inhibitors that can form a covalent bond with an adjacent Cys residue are designed by adding a warhead group to a substitutable position on the reversible inhibitor. For example, a warhead can be bonded to an unsaturated carbon atom that is adjacent to a Cys residue in the target polypeptide. In another example, in the reversible inhibitor target polypeptide complex, a Cys residue is occluded or partly occluded by a portion of the reversible inhibitor. In this situation, a portion of the reversible inhibitor can be removed and replaced with a suitable warhead to produce an inhibitor that covalently binds the Cys residue that is occluded or partially occluded by the reversible inhibitor. This approach is suitable when the portion of the reversible inhibitor that is removed and replaced with the warhead, can be removed without affecting binding of the reversible inhibitor. Portions of a reversible inhibitor that can be removed without affecting binding can be readily identified, and include, for example, portions that are not involved in hydrogen bonding, van der Waals interactions and/or hydrophobic interactions with the target polypeptide.

The warhead comprises a reactive chemical functionality that can react with the Cys side chain to form a covalent bond between the reactive chemical functionality and the sulfur atom of the Cys side chain. The warhead optionally contains a linker that positions the reactive chemical functionality within bonding distance of a Cys side chain in the target polypeptide binding site. The warhead can be selected based on the desired degree of reactivity with the Cys side chain. When present, the linker serves to position the reactive chemical functionality within bonding distance of the target Cys residue. For example, when the adjacent Cys residue is too far from the reversible inhibitor for the reactive chemical functionality to be directly bonded to a substitutable position of the reversible inhibitor, the reactive chemical functionality can be bonded to the substitutable position of the reversible inhibitor through a suitable linker, such as a bivalent C₁ to C₁₈ saturated or unsaturated, straight or branched, hydrocarbon chain.

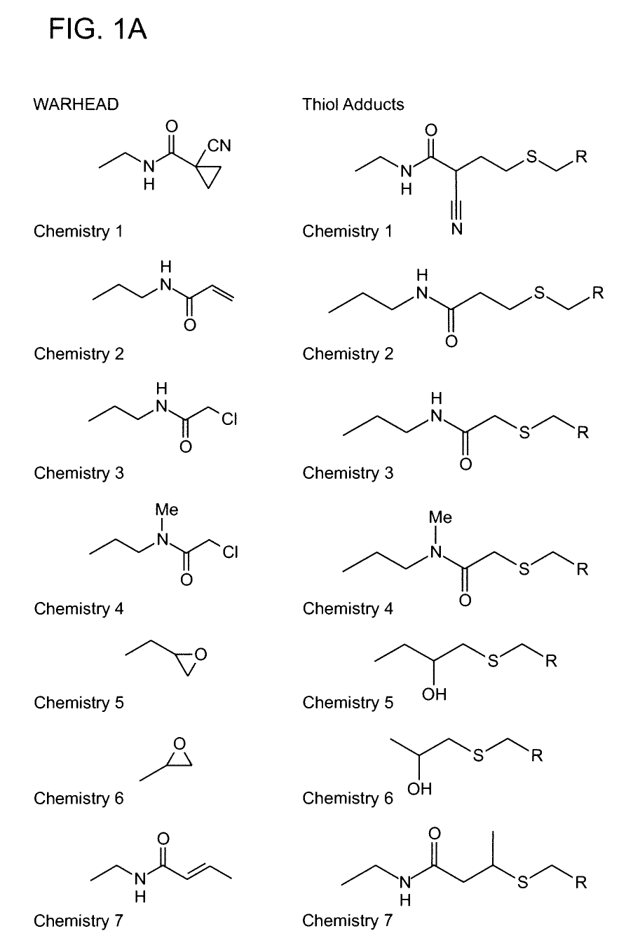

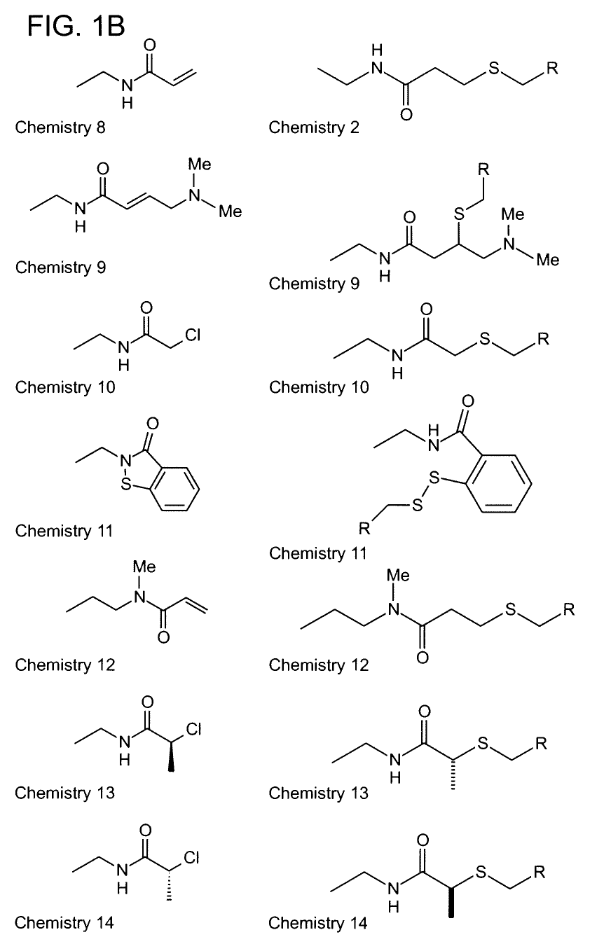

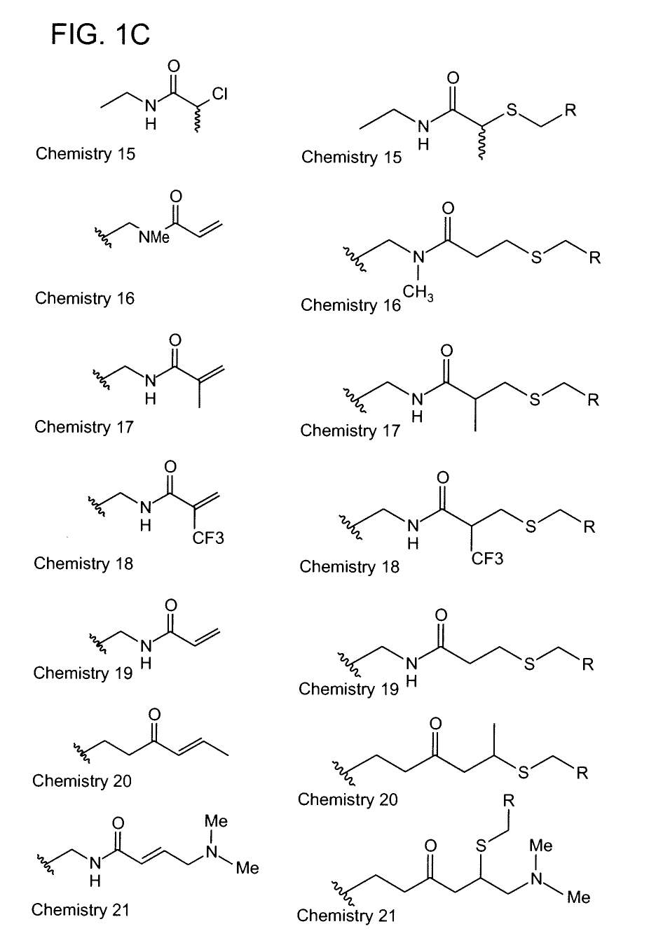

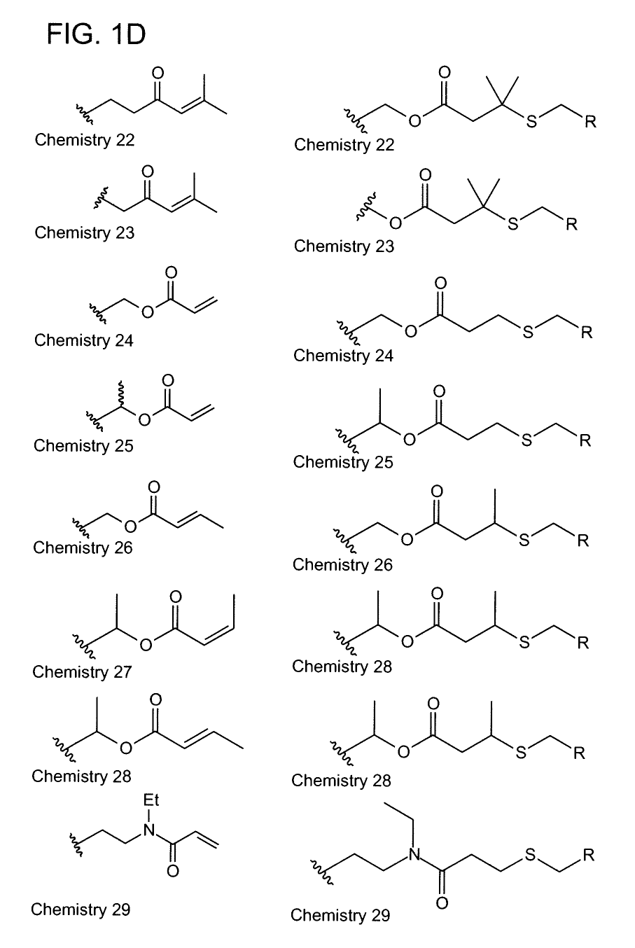

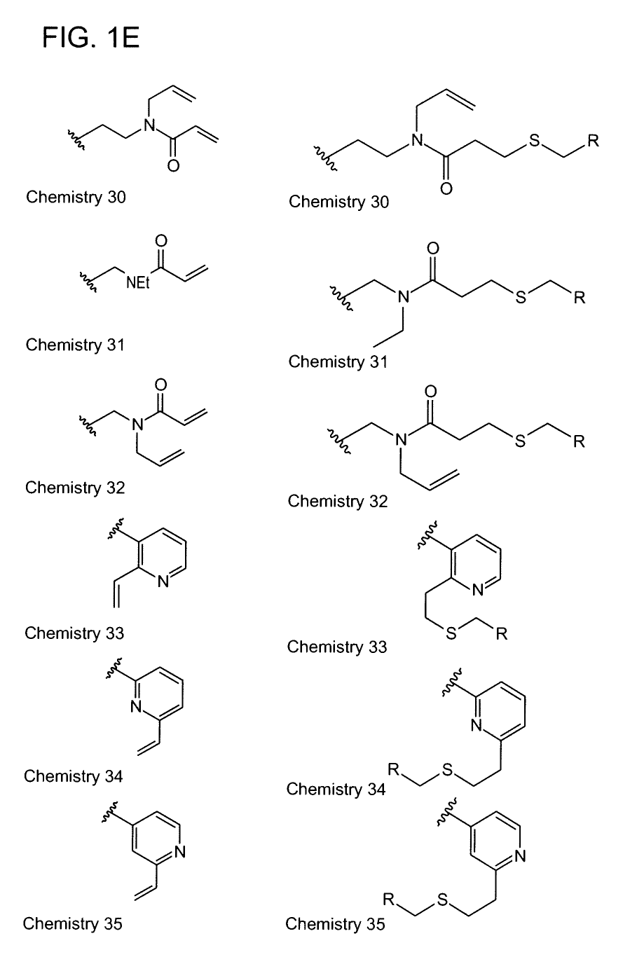

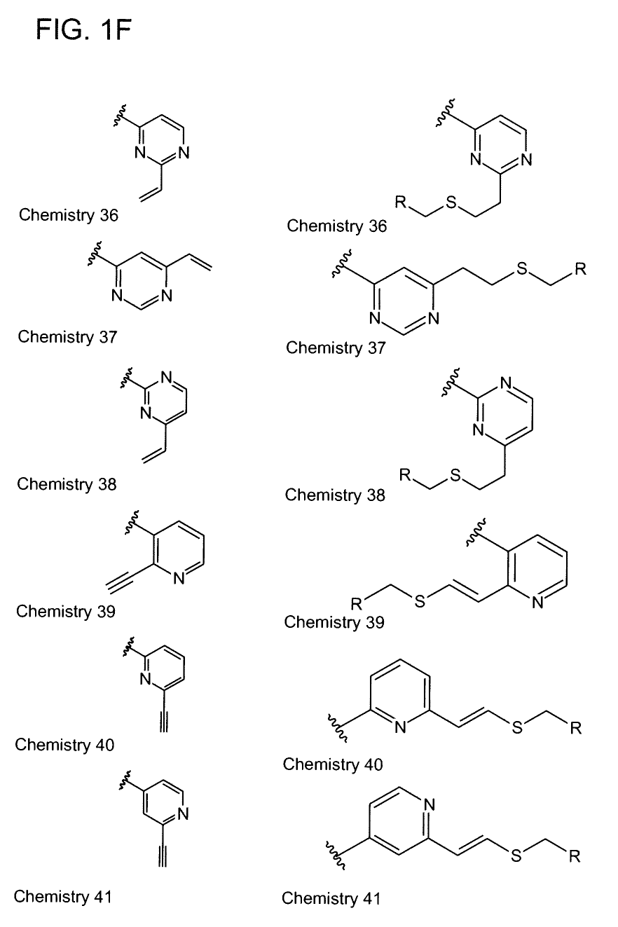

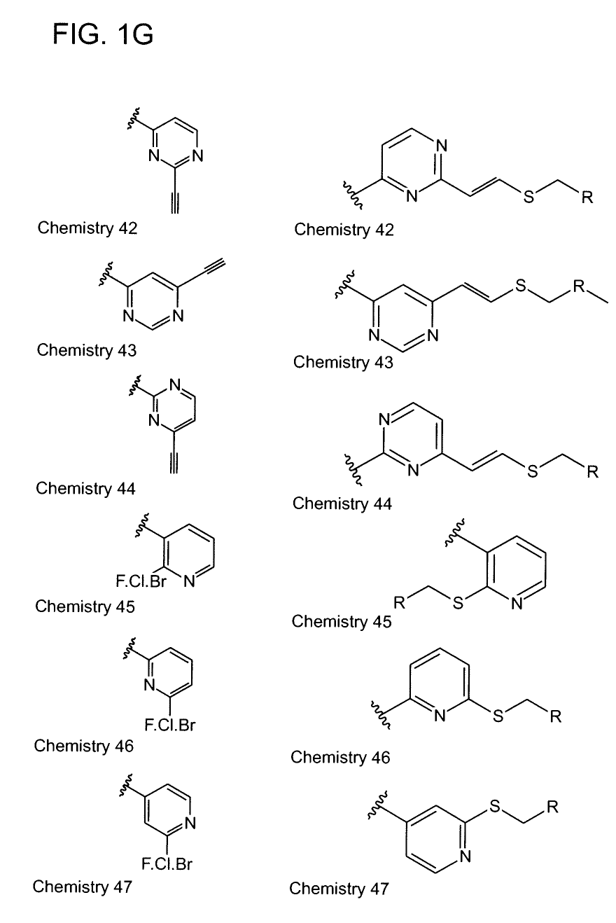

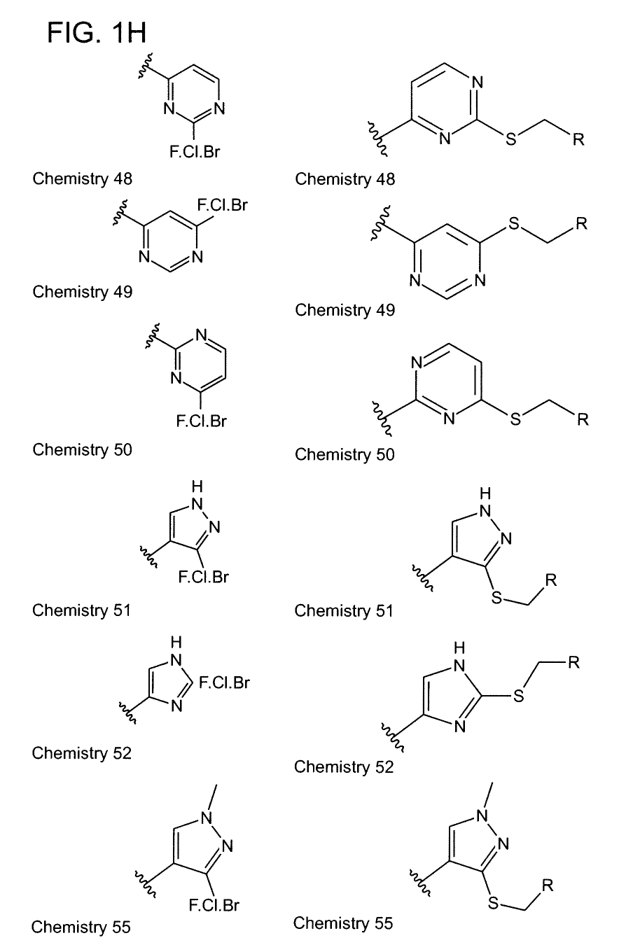

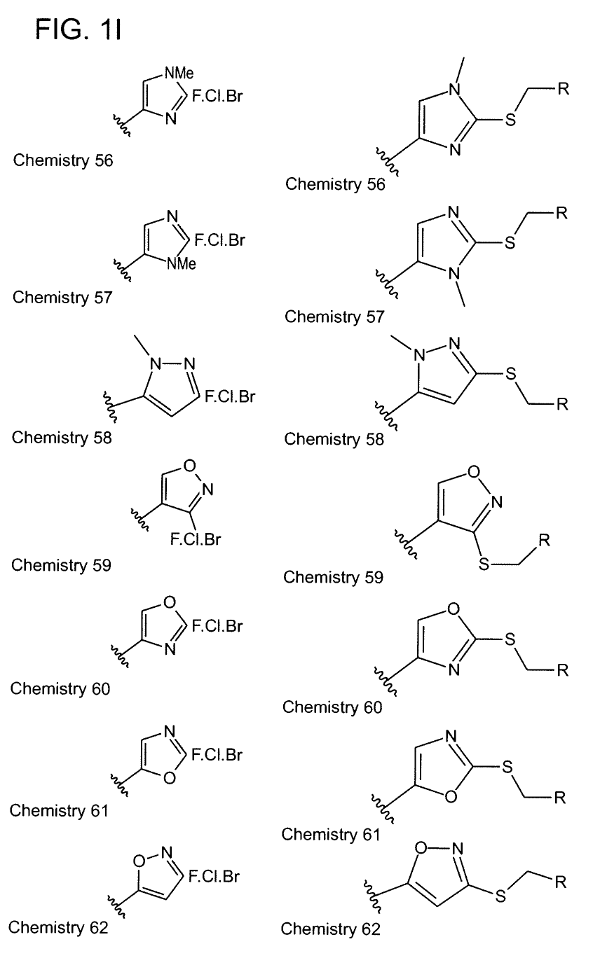

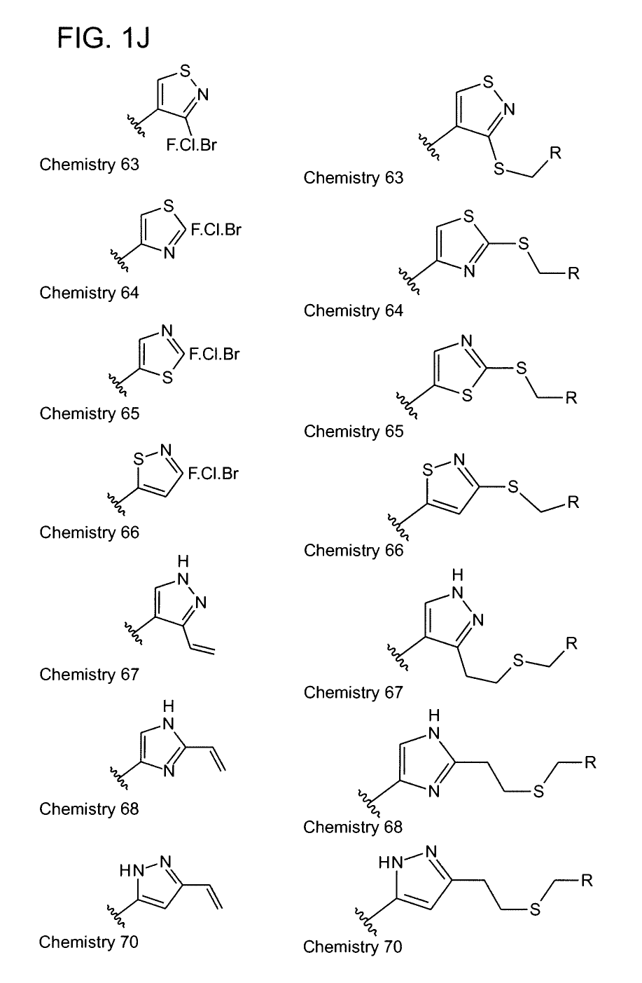

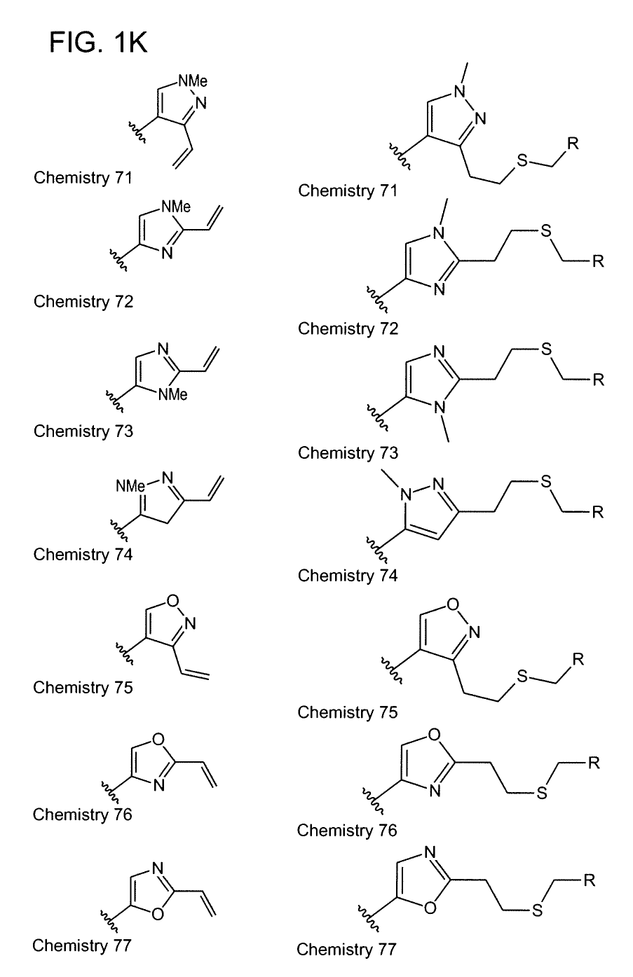

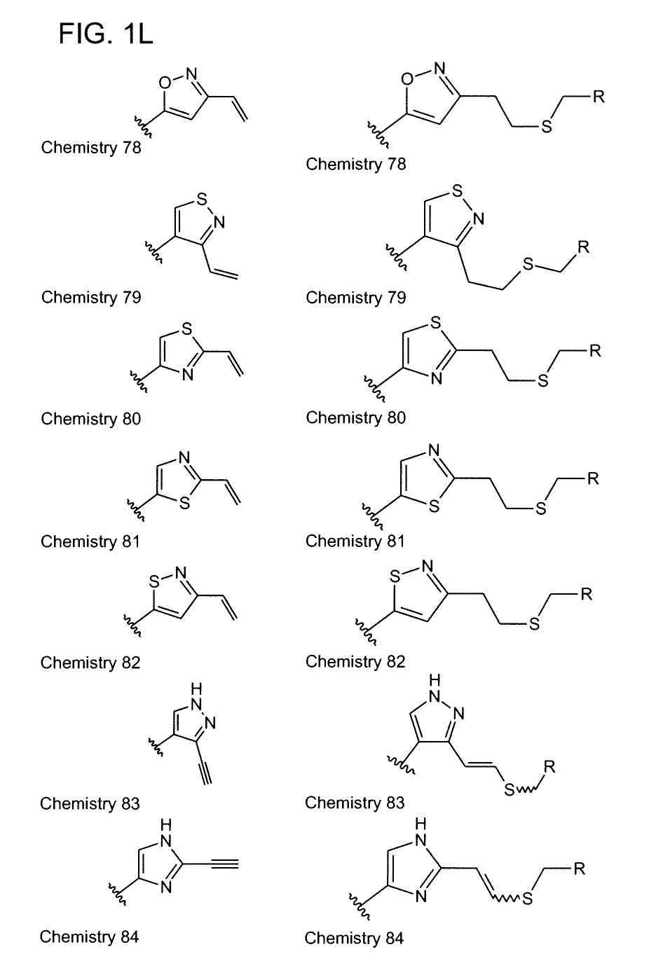

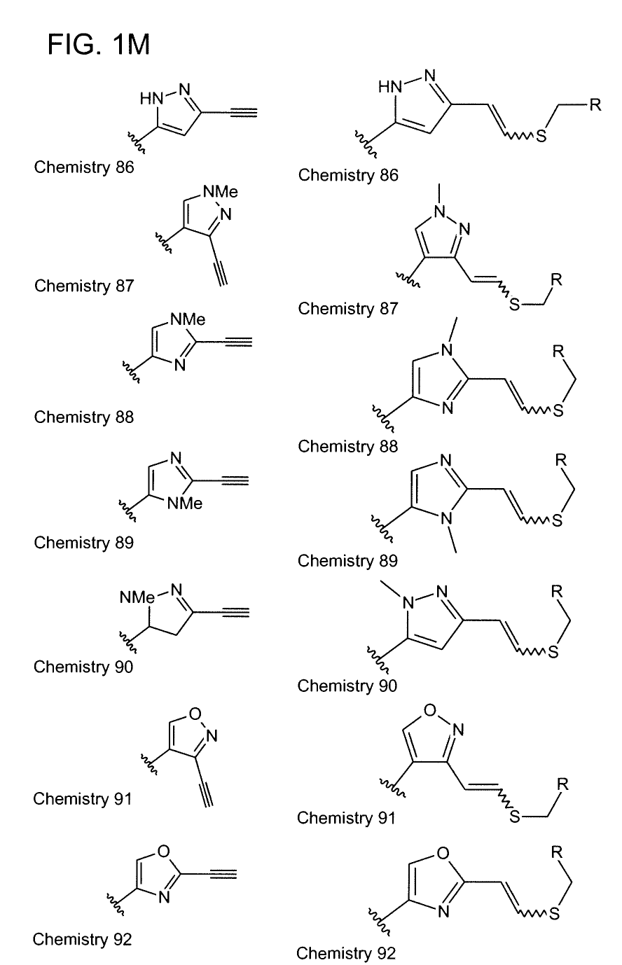

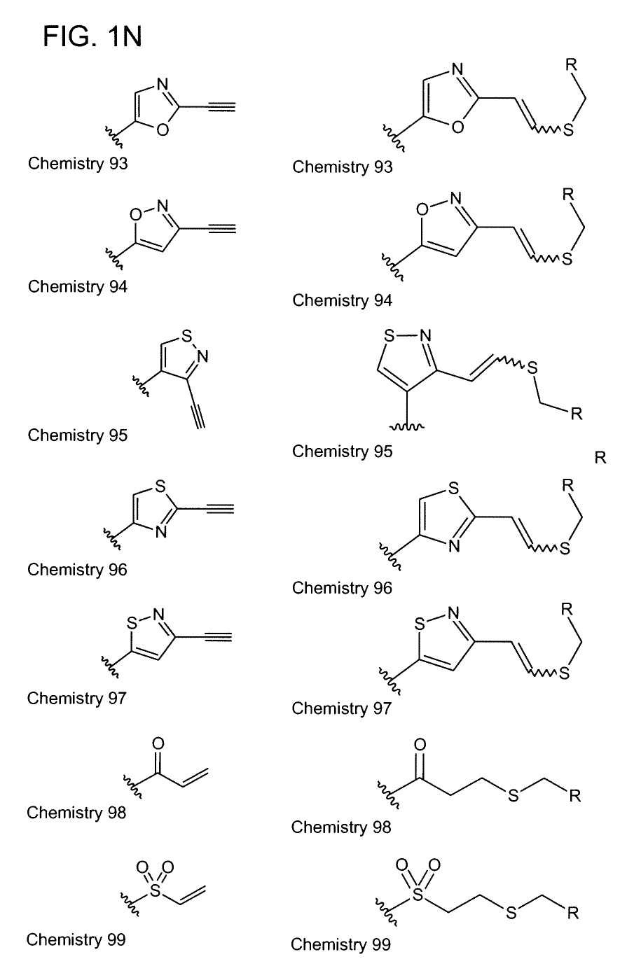

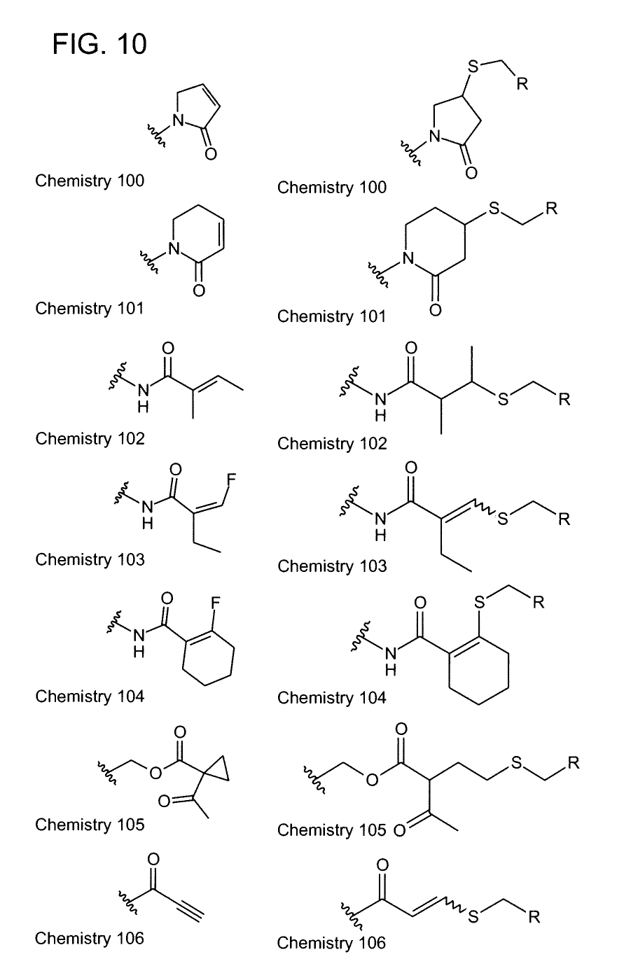

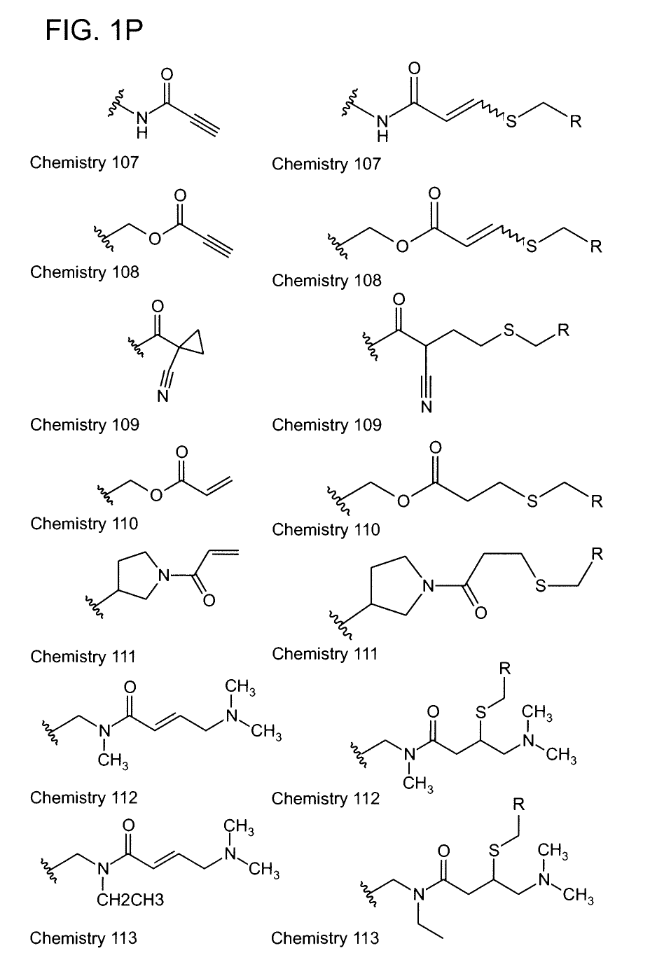

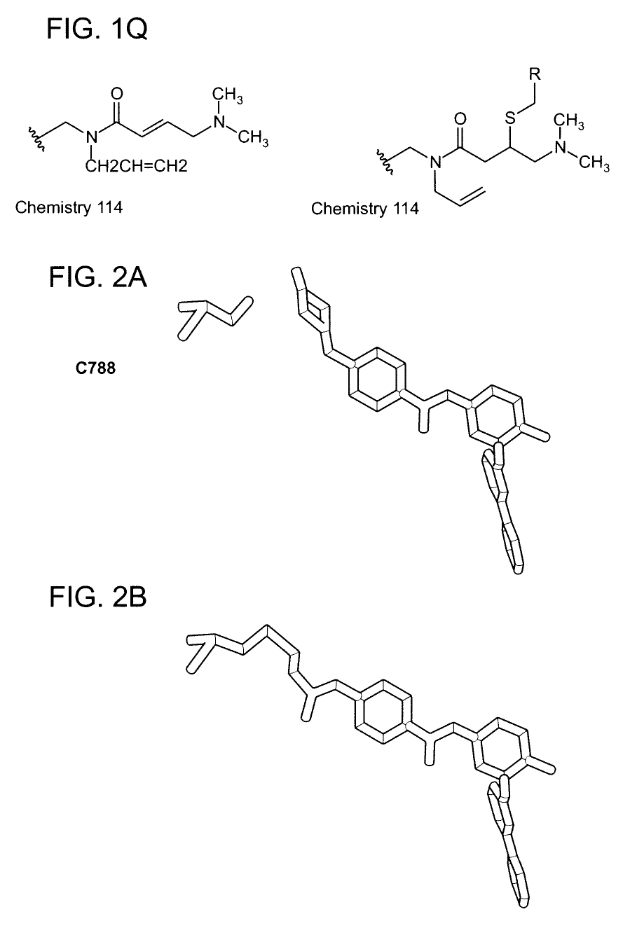

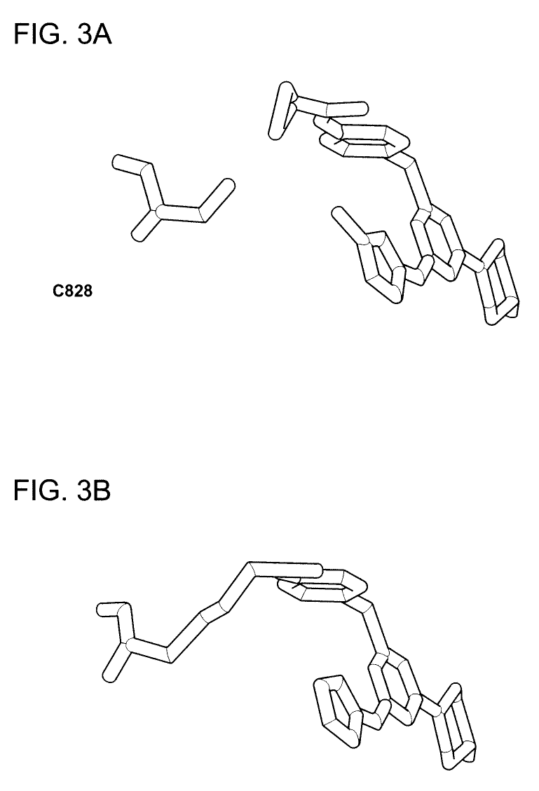

Suitable examples of warhead include those disclosed herein, for example in FIG. 1. Some suitable warheads have the formula *—X-L-Y, wherein * indicates the point of attachment to the substitutable position of the reversible inhibitor.

X is a bond or a bivalent C₁-C₆ saturated or unsaturated, straight or branched hydrocarbon chain wherein optionally one, two or three methylene units of the hydrocarbon chain are independently replaced by NR—, —O—, —C(O)—, —OC(O)—, —C(O)O—, —S—, —SO—, —SO₂—, —C(═S)—, —C(═NR)—, —N═N—, or —C(═N₂)—;

L is a covalent bond or a bivalent C₁₋₈ saturated or unsaturated, straight or branched, hydrocarbon chain, wherein one, two, or three methylene units of L are optionally and independently replaced by cyclopropylene, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—;

Y is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 R^{e} groups; and

each R^{e} is independently selected from -Q-Z, oxo, NO₂, halogen, CN, a suitable leaving group, or a C₁₋₆ aliphatic optionally substituted with oxo, halogen, NO₂, or CN, wherein:

Q is a covalent bond or a bivalent C₁₋₆ saturated or unsaturated, straight or branched, hydrocarbon chain, wherein one or two methylene units of Q are optionally and independently replaced by N(R)—, —S—, —O—, —C(O)—, —OC(O)—, —C(O)O—, —SO—, or —SO₂—, —N(R)C(O)—, —C(O)N(R)—, —N(R)SO₂—, or SO₂N(R)—; and

Z is hydrogen or C₁₋₆ aliphatic optionally substituted with oxo, halogen, NO₂, or CN.

In some embodiments X is a bond, —O—, —NH—, —S—, —O—CH₂—CH₂—O—, O—(CH₂)₃—, or —O—(CH₂)₂—C(CH₃)₂—.

In certain embodiments, L is a covalent bond.

In certain embodiments, L is a bivalent C₁₋₈ saturated or unsaturated, straight or branched, hydrocarbon chain. In certain embodiments, L is —CH₂—.

In certain embodiments, L is a covalent bond, —CH₂—, —NH—, —CH₂NH—, —NHCH₂—, —NHC(O)—, —NHC(O)CH₂OC(O)—, —CH₂NHC(O)—, —NHSO₂—, —NHSO₂CH₂—, —NHC(O)CH₂OC(O)—, or —SO₂NH—.

In some embodiments, L is a bivalent C₂₋₈ straight or branched, hydrocarbon chain wherein L has at least one double bond and one or two additional methylene units of L are optionally and independently replaced by —NRC(O)—, —C(O)NR—, —N(R)SO₂—, —SO₂N(R)—, S—, —S(O)—, —SO₂—, —OC(O)—, C(O)O—, cyclopropylene, —O—, —N(R)—, or —C(O)—.

In certain embodiments, L is a bivalent C₂₋₈ straight or branched, hydrocarbon chain wherein L has at least one double bond and at least one methylene unit of L is replaced by —C(O)—, —NRC(O)—, —C(O)NR—, —N(R)SO₂—, —SO₂N(R)—, S—, —S(O)—, —SO₂—, —OC(O)—, or —C(O)O—, and one or two additional methylene units of L are optionally and independently replaced by cyclopropylene, —O—, —N(R)—, or —C(O)—.

In some embodiments, L is a bivalent C₂₋₈ straight or branched, hydrocarbon chain wherein L has at least one double bond and at least one methylene unit of L is replaced by —C(O)—, and one or two additional methylene units of L are optionally and independently replaced by cyclopropylene, —O—, —N(R)—, or —C(O)—.

As described above, in certain embodiments, L is a bivalent C₂₋₈ straight or branched, hydrocarbon chain wherein L has at least one double bond. One of ordinary skill in the art will recognize that such a double bond may exist within the hydrocarbon chain backbone or may be “exo” to the backbone chain and thus forming an alkylidene group. By way of example, such an L group having an alkylidene branched chain includes —CH₂C(═CH₂)CH₂—. Thus, in some embodiments, L is a bivalent C₂₋₈ straight or branched, hydrocarbon chain wherein L has at least one alkylidenyl double bond. Exemplary L groups include —NHC(O)C(═CH₂)CH₂—.

In certain embodiments, L is a bivalent C₂₋₈ straight or branched, hydrocarbon chain wherein L has at least one double bond and at least one methylene unit of L is replaced by —C(O)—. In certain embodiments, L is —C(O)CH═CH(CH₃)—, —C(O)CH═CHCH₂NH(CH₃)—, —C(O)CH═CH(CH₃)—, —C(O)CH═CH—, —CH₂C(O)CH═CH—, —CH₂C(O)CH═CH(CH₃)—, —CH₂CH₂C(O)CH═CH—, —CH₂CH₂C(O)CH═CHCH₂—, —CH₂CH₂C(O)CH═CHCH₂NH(CH₃)—, or —CH₂CH₂C(O)CH═CH(CH₃)—, or —CH(CH₃)OC(O)CH═CH—.

In certain embodiments, L is a bivalent C₂₋₈ straight or branched, hydrocarbon chain wherein L has at least one double bond and at least one methylene unit of L is replaced by —OC(O)—.

In some embodiments, L is a bivalent C₂₋₈ straight or branched, hydrocarbon chain wherein L has at least one double bond and at least one methylene unit of L is replaced by —NRC(O)—, —C(O)NR—, —N(R)SO₂—, —SO₂N(R)—, S—, —S(O)—, —SO₂—, —OC(O)—, or —C(O)O—, and one or two additional methylene units of L are optionally and independently replaced by cyclopropylene, —O—, —N(R)—, or —C(O)—. In some embodiments, L is CH₂OC(O)CH═CHCH₂—, —CH₂—OC(O)CH═CH—, or —CH(CH═CH₂)OC(O)CH═CH—.

In certain embodiments, L is —NRC(O)CH═CH—, —NRC(O)CH═CHCH₂N(CH₃)—, —NRC(O)CH═CHCH₂O—, —CH₂NRC(O)CH═CH—, —NRSO₂CH═CH—, —NRSO₂CH═CHCH₂—, —NRC(O)(C═N₂)C(O)—, —NRC(O)CH═CHCH₂N(CH₃)—, —NRSO₂CH═CH—, —NRSO₂CH═CHCH₂—, —NRC(O)CH═CHCH₂O—, —NRC(O)C(═CH₂)CH₂—, —CH₂NRC(O)—, —CH₂NRC(O)CH═CH—, —CH₂CH₂NRC(O)—, or —CH₂NRC(O)cyclopropylene-, wherein each R is independently hydrogen or optionally substituted C₁₋₆ aliphatic.

In certain embodiments, L is —NHC(O)CH═CH—, —NHC(O)CH═CHCH₂N(CH₃)—, —NHC(O)CH═CHCH₂O—, —CH₂NHC(O)CH═CH—, —NHSO₂CH═CH—, —NHSO₂CH═CHCH₂—, —NHC(O)(C═N₂)C(O)—, —NHC(O)CH═CHCH₂N(CH₃)—, —NHSO₂CH═CH—, —NHSO₂CH═CHCH₂—, —NHC(O)CH═CHCH₂O—, —NHC(O)C(═CH₂)CH₂—, —CH₂NHC(O)—, —CH₂NHC(O)CH═CH—, —CH₂CH₂NHC(O)—, or —CH₂NHC(O)cyclopropylene-.

In some embodiments, L is a bivalent C₂₋₈ straight or branched, hydrocarbon chain wherein L has at least one triple bond. In certain embodiments, L is a bivalent C₂₋₈ straight or branched, hydrocarbon chain wherein L has at least one triple bond and one or two additional methylene units of L are optionally and independently replaced by —NRC(O)—, —C(O)NR—, —S—, —S(O)—, —SO₂—, —C(═S)—, —C(═NR)—, —O—, —N(R)—, or —C(O)—. In some embodiments, L has at least one triple bond and at least one methylene unit of L is replaced by —N(R)—, —N(R)C(O)—, —C(O)—, —C(O)O—, or —OC(O)—, or —O—.

Exemplary L groups include —C≡C—, —C≡CCH₂N(isopropyl)-, —NHC(O)C≡CCH₂CH₂—, —CH₂—C≡C—CH₂—, —C≡CCH₂O—, —CH₂C(O)C≡C—, —C(O)C≡C—, or —CH₂OC(═O)C≡C—.

In certain embodiments, L is a bivalent C₂₋₈ straight or branched, hydrocarbon chain wherein one methylene unit of L is replaced by cyclopropylene and one or two additional methylene units of L are independently replaced by —C(O)—, —NRC(O)—, —C(O)NR—, —N(R)SO₂—, or —SO₂N(R)—. Exemplary L groups include —NHC(O)-cyclopropylene-SO₂— and —NHC(O)-cyclopropylene-.

As defined generally above, Y is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with at 1-4 R^{e} groups, each R^{e} is independently selected from -Q-Z, oxo, NO₂, halogen, CN, or C₁₋₆ aliphatic, wherein Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂—, —N(R)C(O)—, —C(O)N(R)—, —N(R)SO₂—, or —SO₂N(R)—; and, Z is hydrogen or C₁₋₆ aliphatic optionally substituted with oxo, halogen, NO₂, or CN.

In certain embodiments, Y is hydrogen.

In certain embodiments, Y is C₁₋₆ aliphatic optionally substituted with oxo, halogen, NO₂, or CN. In some embodiments, Y is C₂₋₆ alkenyl optionally substituted with oxo, halogen, NO₂, or CN. In other embodiments, Y is C₂₋₆ alkynyl optionally substituted with oxo, halogen, NO₂, or CN. In some embodiments, Y is C₂₋₆ alkenyl. In other embodiments, Y is C₂₋₄ alkynyl.

In other embodiments, Y is C₁₋₆ alkyl substituted with oxo, halogen, NO₂, or CN. Such Y groups include —CH₂F, —CH₂Cl, —CH₂CN, and —CH₂NO₂. In certain embodiments, Y is a saturated 3-6 membered monocyclic ring having 0-3 heteroatoms independently selected from nitrogen, oxygen, or sulfur, wherein Y is substituted with 1-4 R^{e} groups, wherein each R^{e} is as defined above and described herein.

In some embodiments, Y is a saturated 3-4 membered heterocyclic ring having 1 heteroatom selected from oxygen or nitrogen wherein said ring is substituted with 1-2 R^{e} groups, wherein each R^{e} is as defined above and described herein. Exemplary such rings are epoxide and oxetane rings, wherein each ring is substituted with 1-2 R^{e} groups, wherein each R^{e} is as defined above and described herein.

In other embodiments, Y is a saturated 5-6 membered heterocyclic ring having 1-2 heteroatom selected from oxygen or nitrogen wherein said ring is substituted with 1-4 R^{e} groups, wherein each R^{e} is as defined above and described herein. Such rings include piperidine and pyrrolidine, wherein each ring is substituted with 1-4 R^{e} groups, wherein each R^{e} is as defined above and described herein. In certain embodiments, Y is

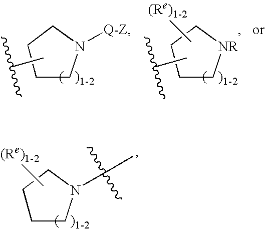

wherein each R, Q, Z, and R^{e} is as defined above and described herein.

In some embodiments, Y is a saturated 3-6 membered carbocyclic ring, wherein said ring is substituted with 1-4 R^{e} groups, wherein each R^{e} is as defined above and described herein. In certain embodiments, Y is cyclopropyl, cyclobutyl, cyclopentyl, or cyclohexyl, wherein each ring is substituted with 1-4 R^{e} groups, wherein each R^{e} is as defined above and described herein. In certain embodiments, Y is

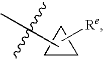

wherein R^{e} is as defined above and described herein. In certain embodiments, Y is cyclopropyl optionally substituted with halogen, CN or NO₂.

In certain embodiments, Y is a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein.

In some embodiments, Y is a partially unsaturated 3-6 membered carbocyclic ring, wherein said ring is substituted with 1-4 R^{e} groups, wherein each R^{e} is as defined above and described herein. In some embodiments, Y is cyclopropenyl, cyclobutenyl, cyclopentenyl, or cyclohexenyl wherein each ring is substituted with 1-4 R^{e} groups, wherein each R^{e} is as defined above and described herein. In certain embodiments, Y is

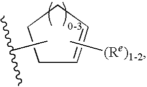

wherein each R^{e} is as defined above and described herein.

In certain embodiments, Y is a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein. In certain embodiments, Y is selected from:

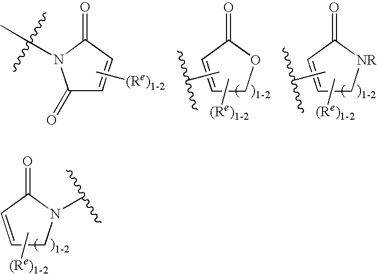

wherein each R and R^{e} is as defined above and described herein.

In certain embodiments, Y is a 6-membered aromatic ring having 0-2 nitrogens wherein said ring is substituted with 1-4 R^{e} groups, wherein each R^{e} group is as defined above and described herein. In certain embodiments, Y is phenyl, pyridyl, or pyrimidinyl, wherein each ring is substituted with 1-4 R^{e} groups, wherein each R^{e} is as defined above and described herein.

In some embodiments, Y is selected from:

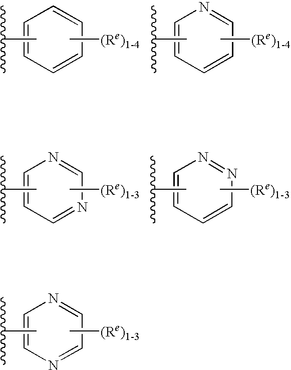

wherein each R^{e} is as defined above and described herein.

In other embodiments, Y is a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein. In some embodiments, Y is a 5 membered partially unsaturated or aryl ring having 1-3 heteroatoms independently selected from nitrogen, oxygen, and sulfur, wherein said ring is substituted with 1-4 R^{e} groups, wherein each R^{e} group is as defined above and described herein. Exemplary such rings are isoxazolyl, oxazolyl, thiazolyl, imidazolyl, pyrazolyl, pyrrolyl, furanyl, thienyl, triazole, thiadiazole, and oxadiazole, wherein each ring is substituted with 1-3 R^{e} groups, wherein each R^{e} group is as defined above and described herein. In certain embodiments, Y is selected from:

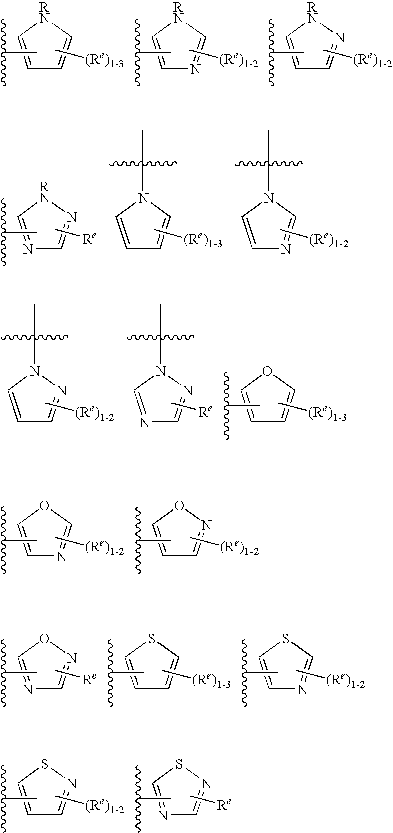

wherein each R and R^{e} is as defined above and described herein.

In certain embodiments, Y is an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein. According to another aspect, Y is a 9-10 membered bicyclic, partially unsaturated, or aryl ring having 1-3 heteroatoms independently selected from nitrogen, oxygen, or sulfur, wherein said ring is substituted with 1-4 R^{e} groups, wherein R^{e} is as defined above and described herein. Exemplary such bicyclic rings include 2,3-dihydrobenzo[d]isothiazole, wherein said ring is substituted with 1-4 R^{e} groups, wherein R^{e} is as defined above and described herein.

As defined generally above, each R^{e} group is independently selected from -Q-Z, oxo, NO₂, halogen, CN, a suitable leaving group, or C₁₋₆ aliphatic optionally substituted with oxo, halogen, NO₂, or CN, wherein Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂—, —N(R)C(O)—, —C(O)N(R)—, —N(R)SO₂—, or —SO₂N(R)—; and Z is hydrogen or C₁₋₆ aliphatic optionally substituted with oxo, halogen, NO₂, or CN.

In certain embodiments, R^{e} is C₁₋₆ aliphatic optionally substituted with oxo, halogen, NO₂, or CN. In other embodiments, R^{e} is oxo, NO₂, halogen, or CN. In some embodiments, R^{e} is -Q-Z, wherein Q is a covalent bond and Z is hydrogen (i.e., R^{e} is hydrogen). In other embodiments, R^{e} is -Q-Z, wherein Q is a bivalent C₁₋₆ saturated or unsaturated, straight or branched, hydrocarbon chain, wherein one or two methylene units of Q are optionally and independently replaced by —NR—, —NRC(O)—, —C(O)NR—, —S—, —O—, —C(O)—, —SO—, or —SO₂—. In other embodiments, Q is a bivalent C₂₋₆ straight or branched, hydrocarbon chain having at least one double bond, wherein one or two methylene units of Q are optionally and independently replaced by —NR—, —NRC(O)—, —C(O)NR—, —S—, —O—, —C(O)—, —SO—, or —SO₂—. In certain embodiments, the Z moiety of the R^{e} group is hydrogen. In some embodiments, -Q-Z is —NHC(O)CH═CH₂ or —C(O)CH═CH₂.

In certain embodiments, each R^{e} is independently selected from oxo, NO₂, CN, fluoro, chloro, —NHC(O)CH═CH₂, —C(O)CH═CH₂, —CH₂CH═CH₂, —C≡CH, —C(O)OCH₂Cl, —C(O)OCH₂F, —C(O)OCH₂CN, —C(O)CH₂Cl, —C(O)CH₂F, —C(O)CH₂CN, or —CH₂C(O)CH₃.

In certain embodiments, R^{e} is a suitable leaving group, ie a group that is subject to nucleophilic displacement. A “suitable leaving group” is a chemical group that is readily displaced by a desired incoming chemical moiety such as the thiol moiety of a cysteine of interest. Suitable leaving groups are well known in the art, e.g., see, “Advanced Organic Chemistry,” Jerry March, 5^{th} Ed., pp. 351-357, John Wiley and Sons, N.Y. Such leaving groups include, but are not limited to, halogen, alkoxy, sulphonyloxy, optionally substituted alkylsulphonyloxy, optionally substituted alkenylsulfonyloxy, optionally substituted arylsulfonyloxy, acyl, and diazonium moieties. Examples of suitable leaving groups include chloro, iodo, bromo, fluoro, acetyl, methanesulfonyloxy (mesyloxy), tosyloxy, triflyloxy, nitro-phenylsulfonyloxy (nosyloxy), and bromo-phenylsulfonyloxy (brosyloxy).

In certain embodiments, the following embodiments and combinations of -L-Y apply:

(a) L is a bivalent C₂₋₈ straight or branched, hydrocarbon chain wherein L has at least one double bond and one or two additional methylene units of L are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—; and Y is hydrogen or C₁₋₆ aliphatic optionally substituted with oxo, halogen, NO₂, or CN; or

(b) L is a bivalent C₂₋₈ straight or branched, hydrocarbon chain wherein L has at least one double bond and at least one methylene unit of L is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L are optionally and independently replaced by cyclopropylene, —O—, —N(R)—, or —C(O)—; and Y is hydrogen or C₁₋₆ aliphatic optionally substituted with oxo, halogen, NO₂, or CN; or

(c) L is a bivalent C₂₋₈ straight or branched, hydrocarbon chain wherein L has at least one double bond and at least one methylene unit of L is replaced by —C(O)—, and one or two additional methylene units of L are optionally and independently replaced by cyclopropylene, —O—, —N(R)—, or —C(O)—; and Y is hydrogen or C₁₋₆ aliphatic optionally substituted with oxo, halogen, NO₂, or CN; or

(d) L is a bivalent C₂₋₈ straight or branched, hydrocarbon chain wherein L has at least one double bond and at least one methylene unit of L is replaced by —C(O)—; and Y is hydrogen or C₁₋₆ aliphatic optionally substituted with oxo, halogen, NO₂, or CN; or

(e) L is a bivalent C₂₋₈ straight or branched, hydrocarbon chain wherein L has at least one double bond and at least one methylene unit of L is replaced by —OC(O)—; and Y is hydrogen or C₁₋₆ aliphatic optionally substituted with oxo, halogen, NO₂, or CN; or

(f) L is —NRC(O)CH═CH—, —NRC(O)CH═CHCH₂N(CH₃)—, —NRC(O)CH═CHCH₂O—, —CH₂NRC(O)CH═CH—, —NRSO₂CH═CH—, —NRSO₂CH═CHCH₂—, —NRC(O)(C═N₂)—, —NRC(O)(C═N₂)C(O)—, —NRC(O)CH═CHCH₂N(CH₃)—, —NRSO₂CH═CH—, —NRSO₂CH═CHCH₂—, —NRC(O)CH═CHCH₂O—, —NRC(O)C(═CH₂)CH₂—, —CH₂NRC(O)—, —CH₂NRC(O)CH═CH—, —CH₂CH₂NRC(O)—, or —CH₂NRC(O)cyclopropylene-; wherein R is H or optionally substituted C₁₋₆ aliphatic; and Y is hydrogen or C₁₋₆ aliphatic optionally substituted with oxo, halogen, NO₂, or CN; or

(g) L is —NHC(O)CH═CH—, —NHC(O)CH═CHCH₂N(CH₃)—, —NHC(O)CH═CHCH₂O—, —CH₂NHC(O)CH═CH—, —NHSO₂CH═CH—, —NHSO₂CH═CHCH₂—, —NHC(O)(C═N₂)—, —NHC(O)(C═N₂)C(O)—, —NHC(O)CH═CHCH₂N(CH₃)—, —NHSO₂CH═CH—, —NHSO₂CH═CHCH₂—, —NHC(O)CH═CHCH₂O—, —NHC(O)C(═CH₂)CH₂—, —CH₂NHC(O)—, —CH₂NHC(O)CH═CH—, —CH₂CH₂NHC(O)—, or —CH₂NHC(O)cyclopropylene-; and Y is hydrogen or C₁₋₆ aliphatic optionally substituted with oxo, halogen, NO₂, or CN; or

(h) L is a bivalent C₂₋₈ straight or branched, hydrocarbon chain wherein L has at least one alkylidenyl double bond and at least one methylene unit of L is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L are optionally and independently replaced by cyclopropylene, —O—, —N(R)—, or —C(O)—; and Y is hydrogen or C₁₋₆ aliphatic optionally substituted with oxo, halogen, NO₂, or CN; or

(i) L is a bivalent C₂₋₈ straight or branched, hydrocarbon chain wherein L has at least one triple bond and one or two additional methylene units of L are optionally and independently replaced by —NRC(O)—, —C(O)NR—, —N(R)SO₂—, —SO₂N(R)—, —S—, —S(O)—, —SO₂—, —OC(O)—, or —C(O)O—, and Y is hydrogen or C₁₋₆ aliphatic optionally substituted with oxo, halogen, NO₂, or CN; or

(j) L is —C≡C—, —C≡CCH₂N(isopropyl)-, —NHC(O)C≡CCH₂CH₂—, —CH₂—C≡C—CH₂—, —C≡CCH₂O—, —CH₂C(O)C≡C—, —C(O)C≡C—, or —CH₂OC(═O)C≡C—; and Y is hydrogen or C₁₋₆ aliphatic optionally substituted with oxo, halogen, NO₂, or CN; or

(k) L is a bivalent C₂₋₈ straight or branched, hydrocarbon chain wherein one methylene unit of L is replaced by cyclopropylene and one or two additional methylene units of L are independently replaced by —NRC(O)—, —C(O)NR—, —N(R)SO₂—, —SO₂N(R)—, —S—, —S(O)—, —SO₂—, —OC(O)—, or —C(O)O—; and Y is hydrogen or C₁₋₆ aliphatic optionally substituted with oxo, halogen, NO₂, or CN; or

(l) L is a covalent bond and Y is selected from:
(i) C₁₋₆ alkyl substituted with oxo, halogen, NO₂, or CN;
(ii) C₂₋₆ alkenyl optionally substituted with oxo, halogen, NO₂, or CN; or
(iii) C₂₋₆ alkynyl optionally substituted with oxo, halogen, NO₂, or CN; or
(iv) a saturated 3-4 membered heterocyclic ring having 1 heteroatom selected from oxygen or nitrogen wherein said ring is substituted with 1-2 R^{e} groups, wherein each R^{e} is as defined above and described herein; or
(v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

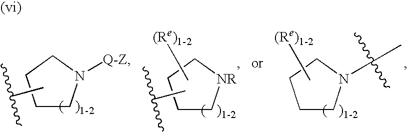

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or

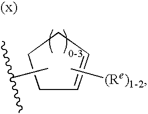

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

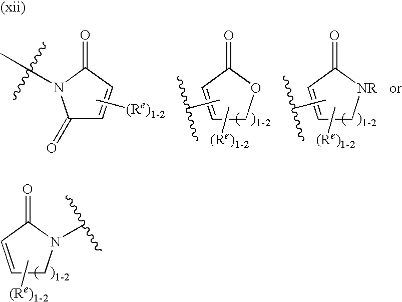

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

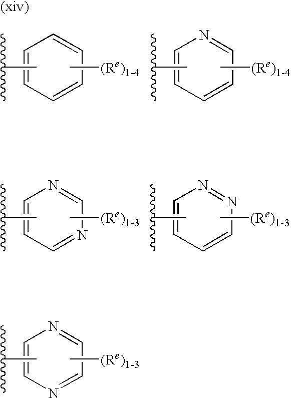

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

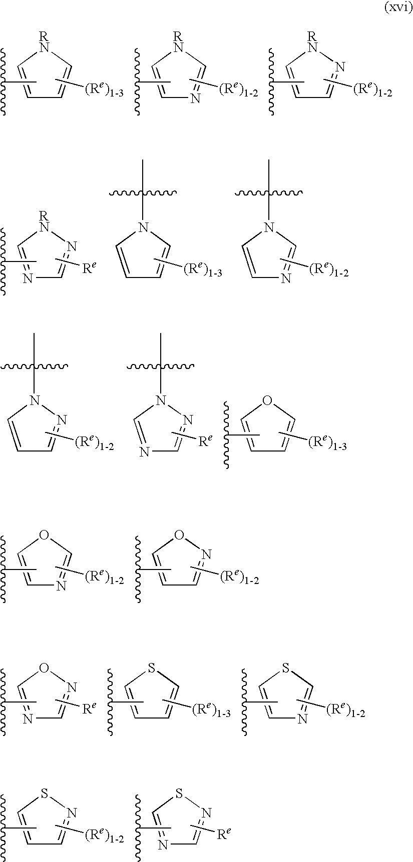

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;

(m) L is —C(O)— and Y is selected from:
(i) C₁₋₆ alkyl substituted with oxo, halogen, NO₂, or CN; or
(ii) C₂₋₆ alkenyl optionally substituted with oxo, halogen, NO₂, or CN; or
(iii) C₂₋₆ alkynyl optionally substituted with oxo, halogen, NO₂, or CN; or
(iv) a saturated 3-4 membered heterocyclic ring having 1 heteroatom selected from oxygen or nitrogen wherein said ring is substituted with 1-2 R^{e} groups, wherein each R^{e} is as defined above and described herein; or
(v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

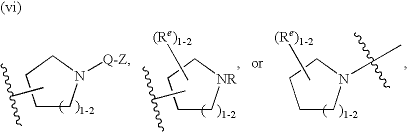

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein;

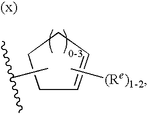

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

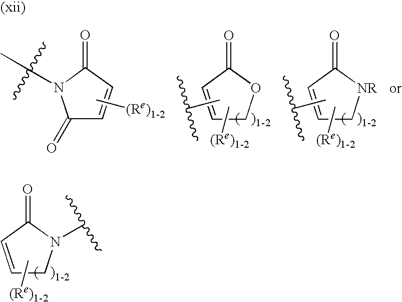

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

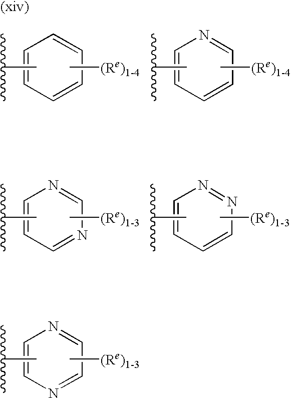

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

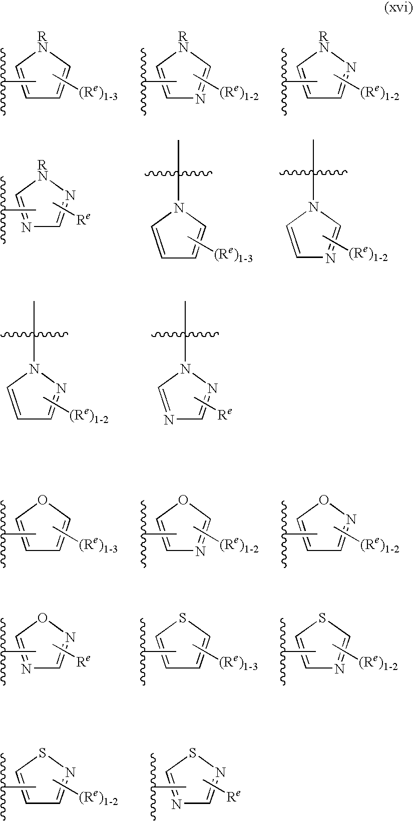

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;

(n) L is —N(R)C(O)— and Y is selected from:
(i) C₁₋₆ alkyl substituted with oxo, halogen, NO₂, or CN; or
(ii) C₂₋₆ alkenyl optionally substituted with oxo, halogen, NO₂, or CN; or
(iii) C₂₋₆ alkynyl optionally substituted with oxo, halogen, NO₂, or CN; or
(iv) a saturated 3-4 membered heterocyclic ring having 1 heteroatom selected from oxygen or nitrogen wherein said ring is substituted with 1-2 R^{e} groups, wherein each R^{e} is as defined above and described herein; or
(v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

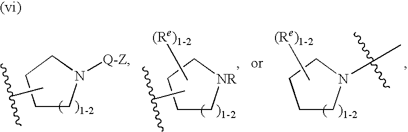

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein;

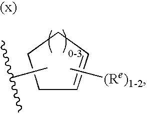

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

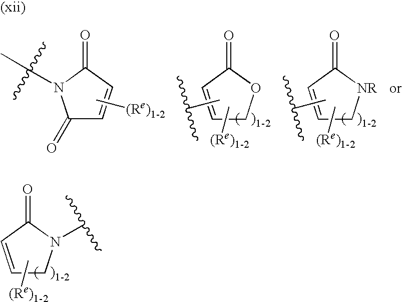

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

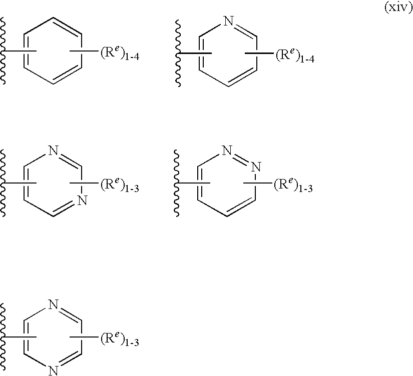

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

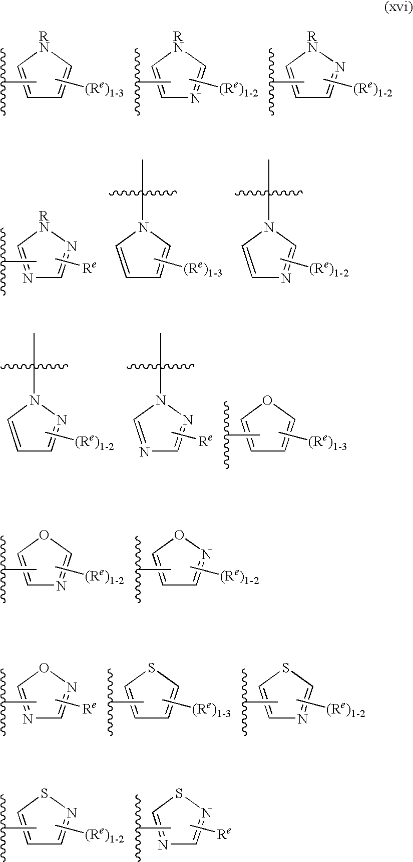

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;

(o) L is a bivalent C₁₋₈ saturated or unsaturated, straight or branched, hydrocarbon chain; and Y is selected from:
(i) C₁₋₆ alkyl substituted with oxo, halogen, NO₂, or CN;
(ii) C₂₋₆ alkenyl optionally substituted with oxo, halogen, NO₂, or CN; or
(iii) C₂₋₆ alkynyl optionally substituted with oxo, halogen, NO₂, or CN; or
(iv) a saturated 3-4 membered heterocyclic ring having 1 heteroatom selected from oxygen or nitrogen wherein said ring is substituted with 1-2 R^{e} groups, wherein each R^{e} is as defined above and described herein; or
(v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

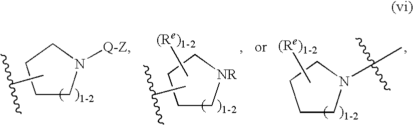

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein;

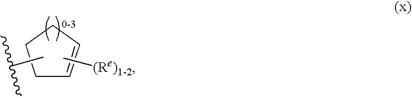

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

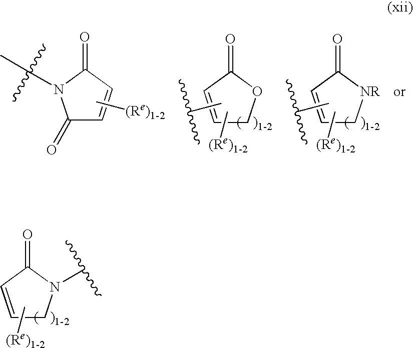

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

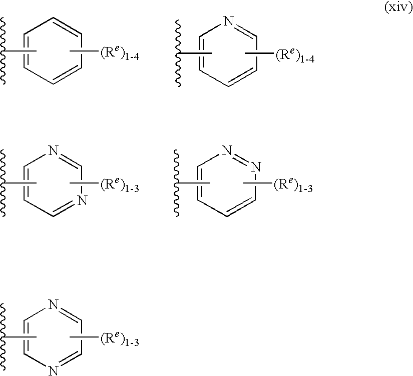

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

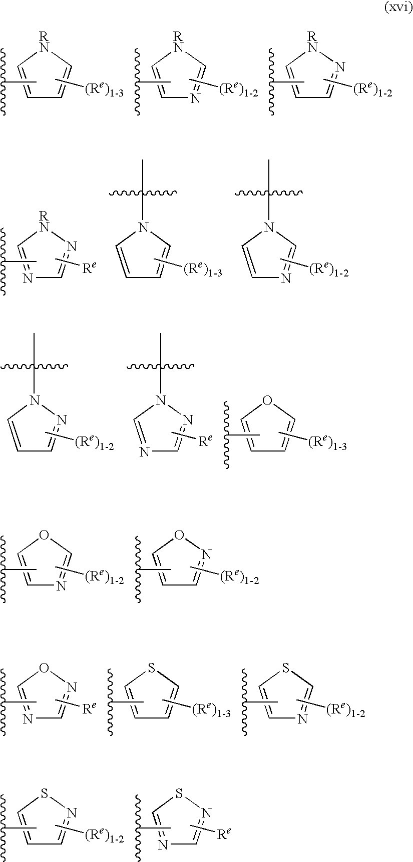

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;

(p) L is a covalent bond, —CH₂—, —NH—, —C(O)—, —CH₂NH—, —NHCH₂—, —NHC(O)—, —NHC(O)CH₂OC(O)—, —CH₂NHC(O)—, —NHSO₂—, —NHSO₂CH₂—, —NHC(O)CH₂OC(O)—, or —SO₂NH—; and Y is selected from:
(i) C₁₋₆ alkyl substituted with oxo, halogen, NO₂, or CN; or
(ii) C₂₋₆ alkenyl optionally substituted with oxo, halogen, NO₂, or CN; or
(iii) C₂₋₆ alkynyl optionally substituted with oxo, halogen, NO₂, or CN; or
(iv) a saturated 3-4 membered heterocyclic ring having 1 heteroatom selected from oxygen or nitrogen wherein said ring is substituted with 1-2 R^{e} groups, wherein each R^{e} is as defined above and described herein; or
(v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

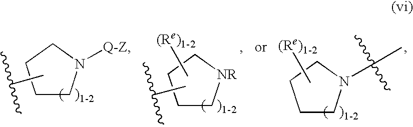

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein;

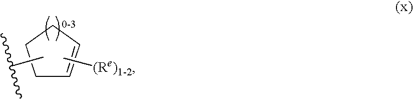

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

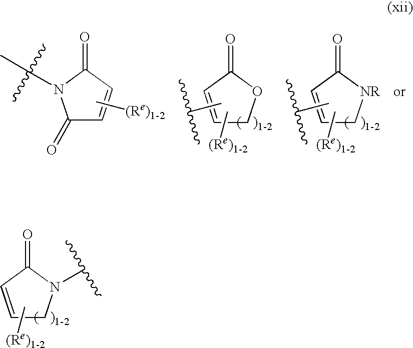

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

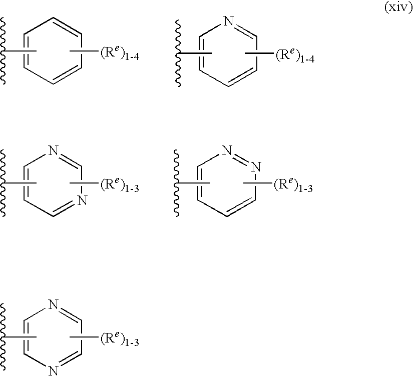

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

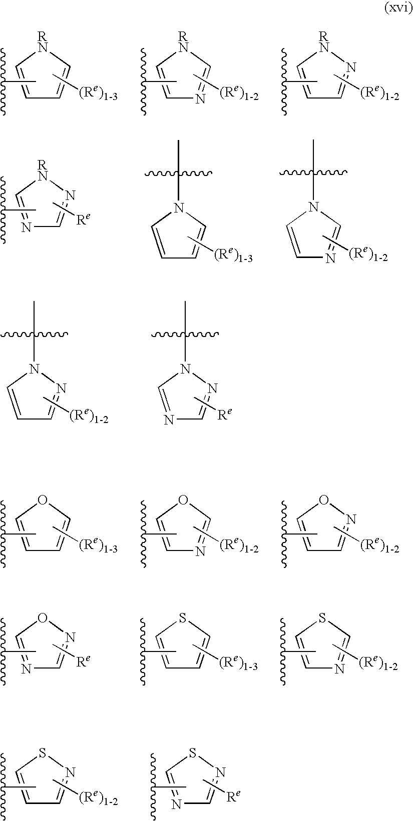

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein.

In certain embodiments, the Y group of formula I is selected from those set forth in Table 3, below, wherein each wavy line indicates the point of attachment to the rest of the molecule. Each R^{e} group depicted in Table 2 is independently selected from halogen.

TABLE 3
Exemplary Y groups:

| | |
|---|---|
| 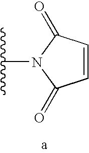 | 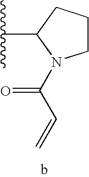 |
| 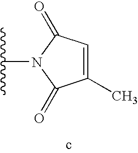 | 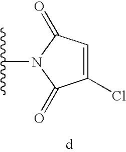 |
| 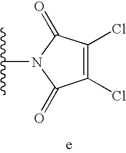 | 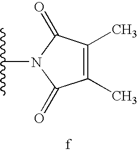 |
| 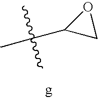 | 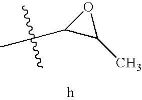 |
| 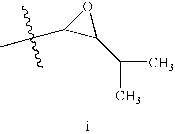 | 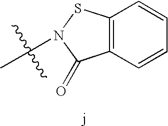 |
| 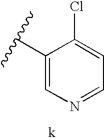 | 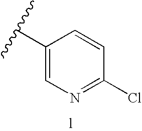 |
| 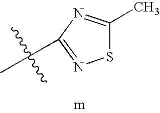 | 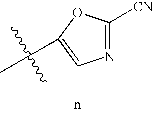 |
| 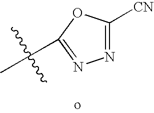 | 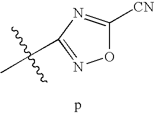 |
| 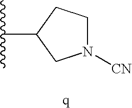 | 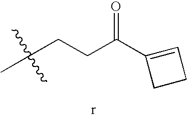 |
| 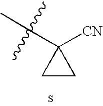 | 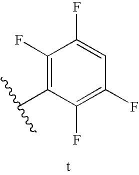 |
| 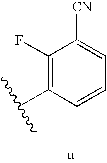 | 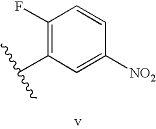 |
| 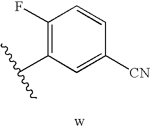 | 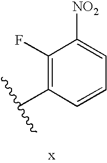 |
| 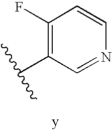 | 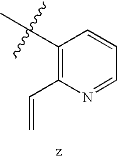 |
| 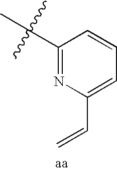 | 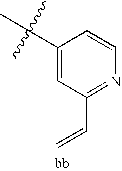 |
| 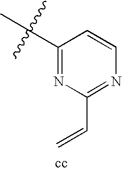 | 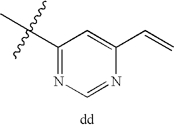 |
| 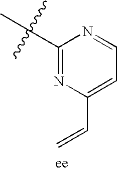 | 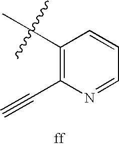 |
| 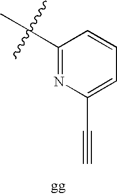 | 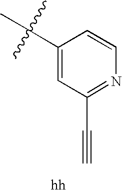 |
| 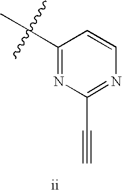 | 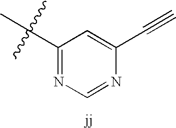 |
| 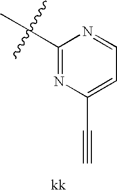 | 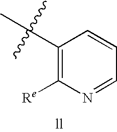 |
| 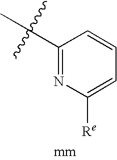 | 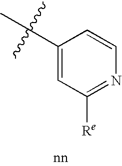 |
| 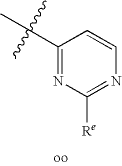 | 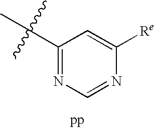 |
| 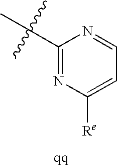 | 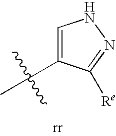 |
| 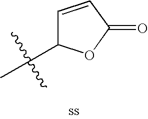 | 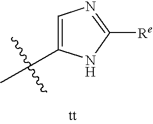 |
| 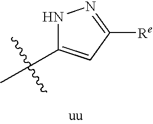 | 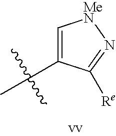 |
| 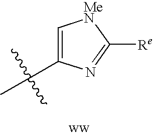 | 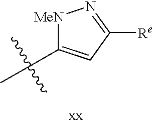 |
| 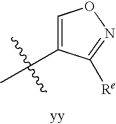 | 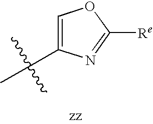 |
| 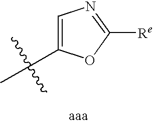 | 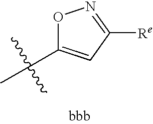 |
| 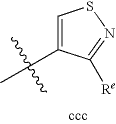 | 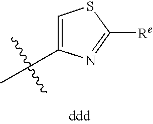 |
| 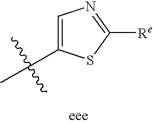 | 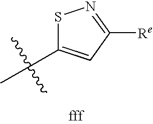 |
| 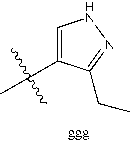 | 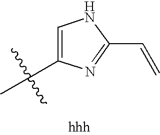 |
| 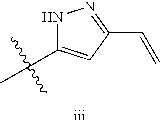 | 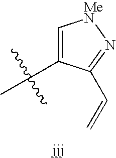 |
| 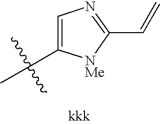 | 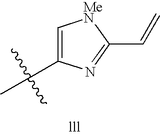 |
| 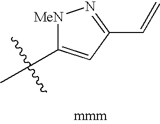 | 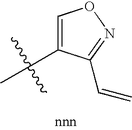 |
| 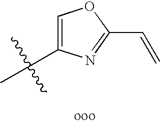 | 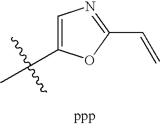 |
| 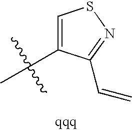 | 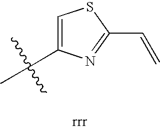 |
| 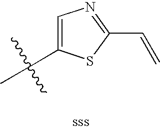 | 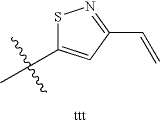 |
| 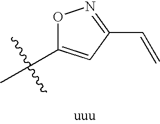 | 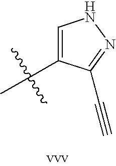 |
| 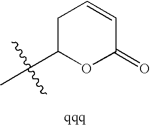 | 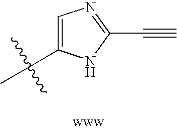 |
| 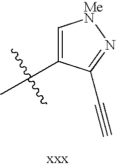 | 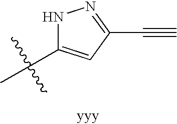 |
| 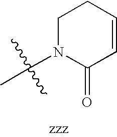 | 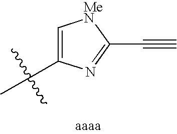 |
| 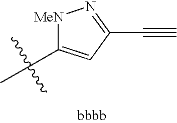 | 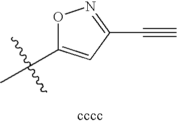 |
| 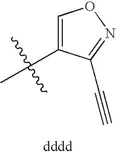 | 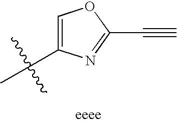 |
| 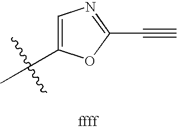 | 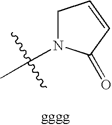 |
| 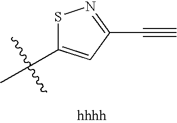 | 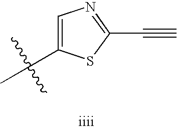 |
| 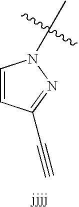 | 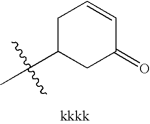 |
| 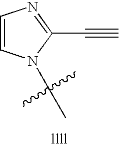 | 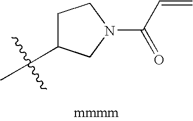 |
| 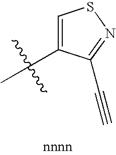 | 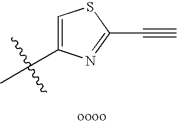 |
| 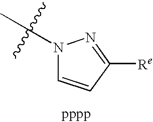 | 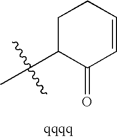 |
| 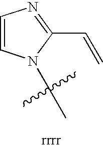 | 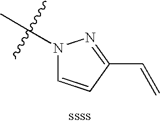 |
| 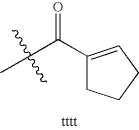 | 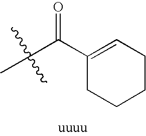 |
| 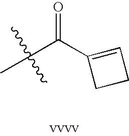 | 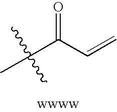 |
| 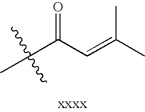 | 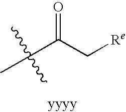 |
| 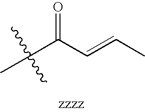 | 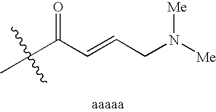 |
| 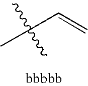 | 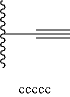 |

In certain embodiments, R¹ is —C≡CH, —C≡CCH₂NH(isopropyl), —NHC(O)C≡CCH₂CH₃, —CH₂—C≡C—CH₃, —C≡CCH₂OH, —CH₂C(O)C≡CH, —C(O)C≡CH, or —CH₂C(═O)C≡CH. In some embodiments, R¹ is selected from —NHC(O)CH═CH₂, —NHC(O)CH═CHCH₂N(CH₃)₂, or —CH₂NHC(O)CH═CH₂.

In certain embodiments, R¹ is selected from those set forth in Table 4, below, wherein each wavy line indicates the point of attachment to the rest of the molecule.

TABLE 4
Exemplary R¹ Groups

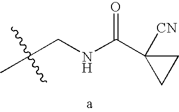

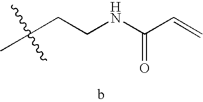

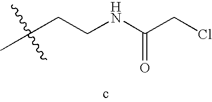

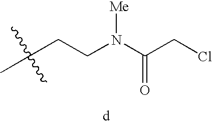

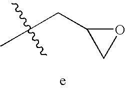

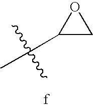

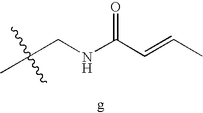

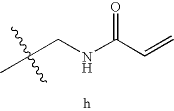

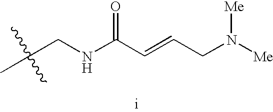

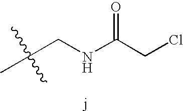

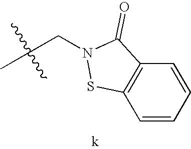

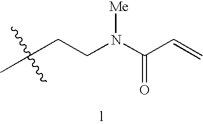

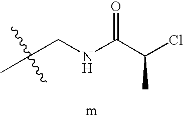

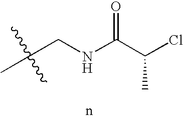

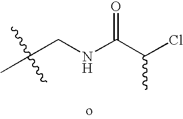

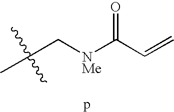

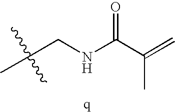

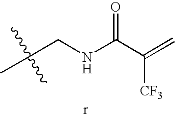

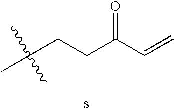

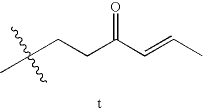

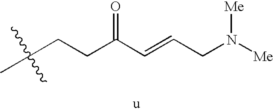

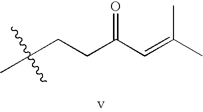

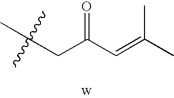

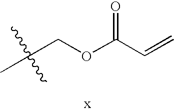

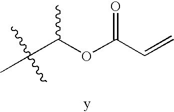

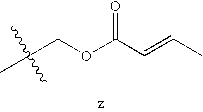

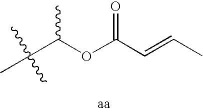

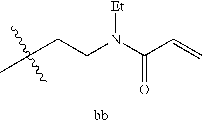

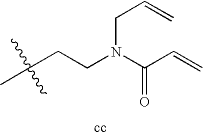

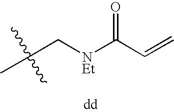

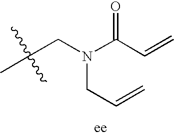

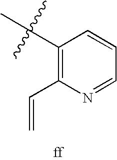

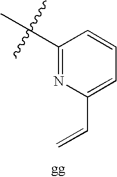

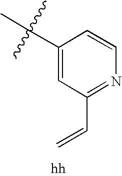

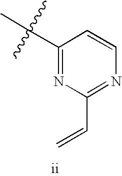

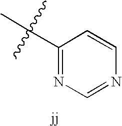

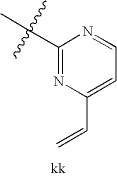

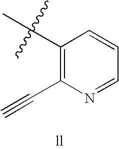

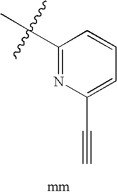

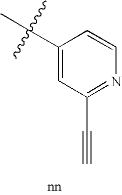

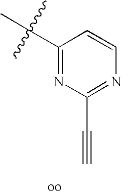

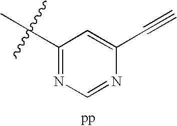

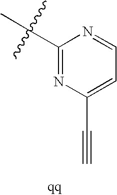

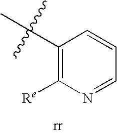

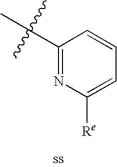

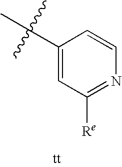

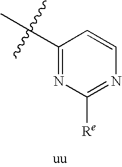

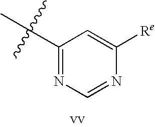

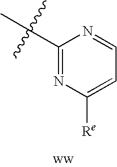

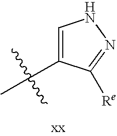

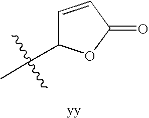

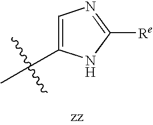

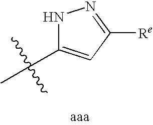

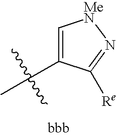

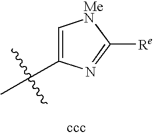

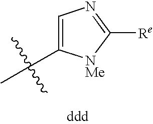

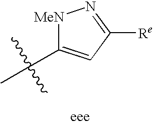

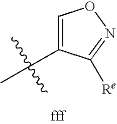

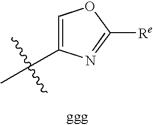

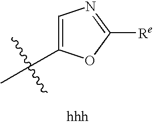

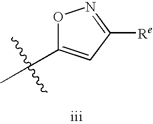

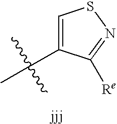

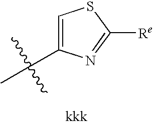

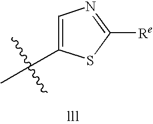

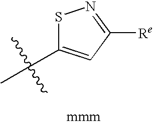

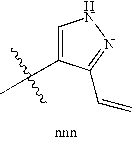

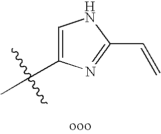

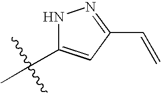

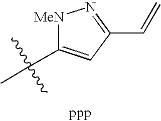

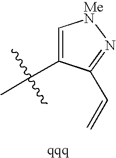

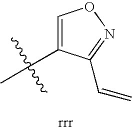

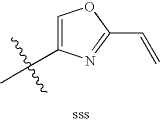

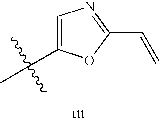

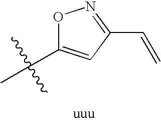

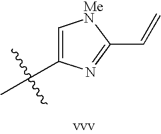

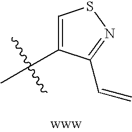

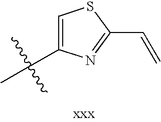

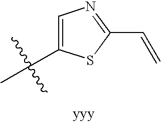

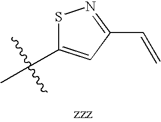

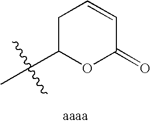

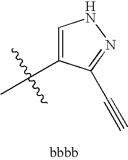

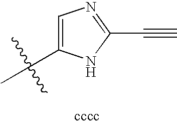

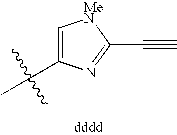

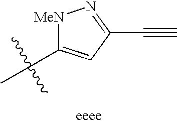

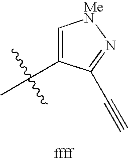

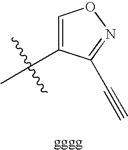

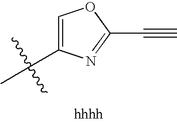

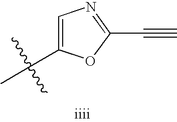

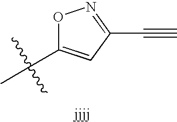

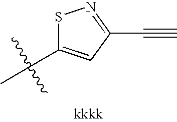

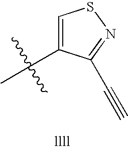

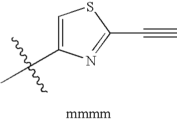

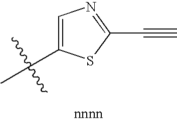

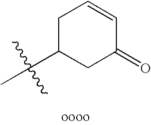

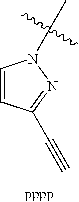

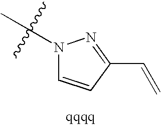

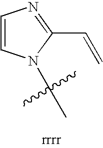

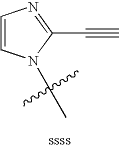

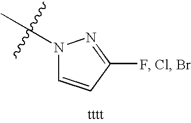

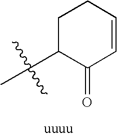

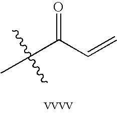

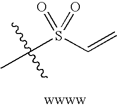

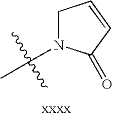

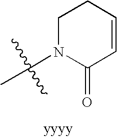

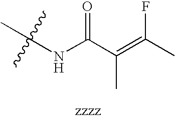

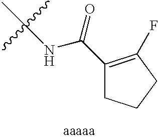

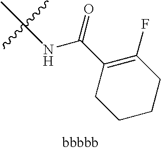

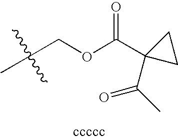

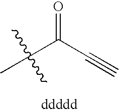

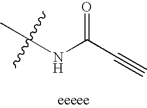

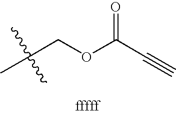

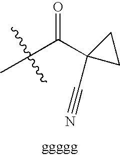

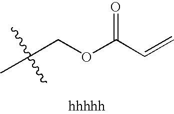

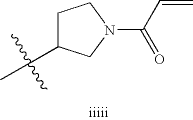

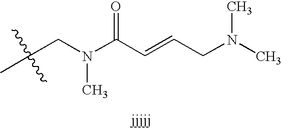

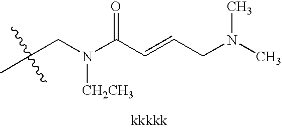

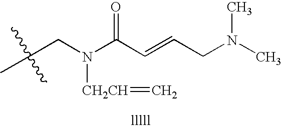

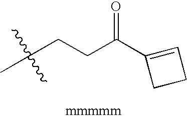

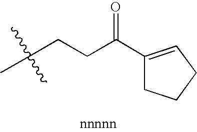

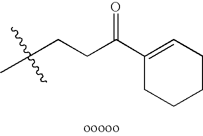

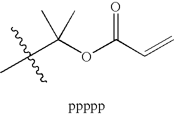

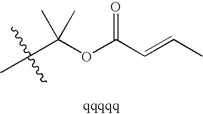

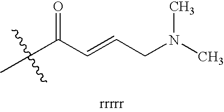

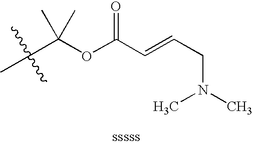

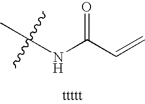

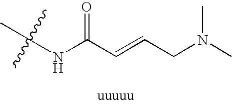

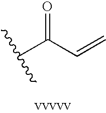

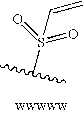

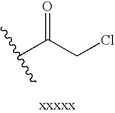

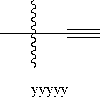

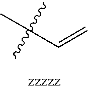

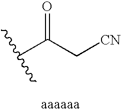

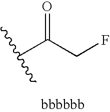

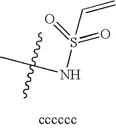

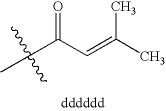

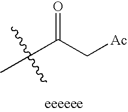

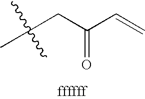

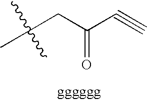

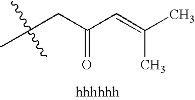

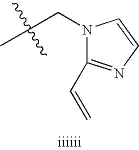

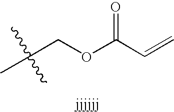

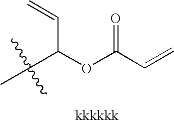

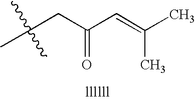

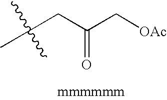

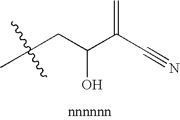

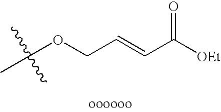

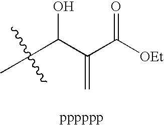

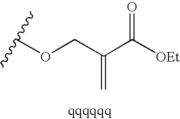

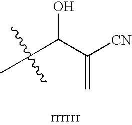

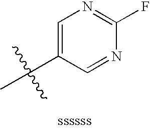

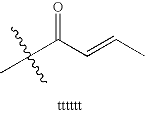

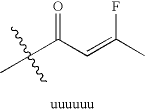

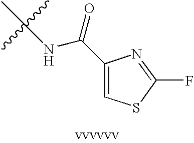

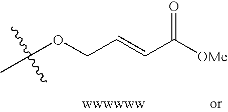

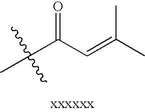

wherein each R^{e} is independently a suitable leaving group, NO₂, CN, or oxo.

Structural models of candidate inhibitors that contain a warhead can be prepared using any suitable method. For example, as described and exemplified herein, warheads can be built in three dimensions onto a reversible inhibitor template using a suitable molecular modeling program. Suitable modeling programs include Discovery Studio® and Pipeline Pilot™ (molecular modeling software, Accelrys Inc., San Diego, Calif.), Combibuild, Combilibmaker 3D, (software for producing compound libraries, Tripos L. P., St. Louis, Mo.), SMOG (small molecule computational combinatorial design program; DeWitte and Shakhnovich, J. Am. Chem. Soc. 118:11733-11744 (1996); DeWitte et al., J. Am. Chem. Soc. 119:4608-4617 (1997); Shimada et al., Protein Sci. 9:765-775 (2000); Maestro™, CombiGlide™, Glide™ and Jaguar™ (Modeling softwar packages, Schrödinger, LLC. 120 West 45th Street, New York, N.Y. 10036-4041)). Warheads can be attached to each substitutable position that is adjacent to a Cys residue in the target polypeptides, or to selected substitutable positions or a single substitutable position as desired. Warheads can be attached to compounds using any suitable method or program, such as FROG (3D conformation generator; Bohme et al., Nucleic Acids Res. 35(web server issue):W568-W572 (2007).), Discovery Studio® or Pipline Pilot™ (Accelrys, Inc., San Diego), Combilibmaker 3D (Tripos, St. Louis), SMOG (DeWitte and Shakhnovich, J. Am. Chem. Soc. 118:11733-11744 (1996); DeWitte et al., J. Am. Chem. Soc. 119:4608-4617 (1997); Shimada et al., Protein Sci. 9:765-775 (2000)), and the like. Warheads can be attached manually, as for example with Discovery Studio®, or in automated fashion, as for example with Pipline Pilot™ (Accelrys, Inc., San Diego).

In some preferred embodiments, structural models of a plurality of candidate inhibitors are produced. The structural models including compounds in which the warhead is attached to a different substitutable position, and attachment to each possible substitutable position is represented by at least one compound.

### D) Determine Proximity of Warhead to Target Cysteine

The invention comprises determining the substitutable positions of the reversible inhibitor that result in the reactive chemical functionality of the warhead being within bonding distance of the Cys residue in the binding site of the target polypeptide when the candidate inhibitor is bound to the binding site. Structural models of candidate inhibitors are analyzed to determine which substitutable positions in the reversible inhibitor result in the reactive chemical functionality of the warhead being within bonding distance of a Cys residue in the binding site of the target polypeptide. Cys residue—substitutable position combinations that result in the reactive chemical functionality being within bonding distance of the Cys residue in the structural model can be identified using any suitable method of determining intermolecular distances with or without constraints. For example, Cys residue—substitutable position combinations that results in the reactive chemical functionality being within bonding distance of the Cys residue can be identified using a suitable computational method in which 1) the target polypeptide is held fixed except the Cys side chain is allowed to flex, and the candidate inhibitor is held fixed except the warhead is allowed to flex, 2) the target polypeptide is allowed to flex and the candidate inhibitor is allowed to flex, 3) the target polypeptide is allowed to flex and the candidate inhibitor is held fixed except the warhead is allowed to flex, or 4) target polypeptide is held fixed except the Cys side chain is allowed to flex, and the candidate inhibitor is allowed to flex. Preferably, the target polypeptide is held fixed except the Cys side chain is allowed to flex, and the candidate inhibitor is held fixed except the warhead is allowed to flex.

Several computational methods that are suitable for identifying Cys residue—substitutable position combinations that results in the reactive chemical functionality being within bonding distance of the Cys residue are well-known in the art. For example, programs that are suitable for computing intermolecular distances, molecular dynamics, energy minimizations, systematic conformational searches and manual modeling, are well known in the art. Suitable programs include, for example, Discovery Studio® and Chammn (Accelrys, Inc. San Diego), Amber (Amber Software Administrator, USSF, 600 16th Street, Room 552, San Fransico, Calif. 94158 and ambermd.org/) and the like. Computer programs that can evaluate compound deformation energy and electrostatic interactions are available in the art and include, for example, Gaussian 92, revision C (M. J. Frisch, Gaussian, Inc., Pittsburgh, Pa.); AMBER, version 4.0 (P. A. Kollman, University of California at San Francisco, Calif.); QUANTA/CHARMM (Accelrys, Inc., Burlington, Mass.). These programs may be implemented, for instance, using a computer workstation. Other suitable hardware systems and software packages are known to those skilled in the art. Docking of candidate inhibitors may be accomplished using suitable software, such as Flexx (Tripos, St. Louis, Mo.), Glide (Schrodinger, New York), ICM-Pro (Molsoft, California) and the like, and followed by energy minimization and molecular dynamics with standard molecular mechanics forcefields, such as OPLS-AA, CHARMM or AMBER.

### E) Form a Covalent Bond

The invention comprises forming a covalent bond between the sulfur atom of the Cys residue in the binding site and the reactive chemical functionality of the warhead. Identifying Cys residue—substitutable position combinations that results in the reactive chemical functionality being within bonding distance of the Cys residue identifies candidate inhibitors that are likely to covalently modify the Cys residue. However, spherical proximity of the reactive chemical functionality and the Cys side chain in the model alone is not a sufficient indicator that a covalent bond will form between the reactive chemical functionality and the Cys side chain. Accordingly, in the algorithm and method of the invention a bond is formed between the reactive chemical functionality and the Cys side chain, and the length of the formed bond is analyzed. A covalent bond length of about 2.1 angstroms to about 1.5 angstroms, or preferably less than about 2 angstroms, for the bond formed between the sulfur atom of the Cys residue in the binding site and the reactive chemical functionality of the warhead, indicates that the candidate inhibitor is an inhibitor that will covalently bind a target polypeptide. Preferably, the length of the bond formed between the reactive chemical functionality and the Cys side chain is about 2 angstroms, about 1.9 angstroms, about 1.8 angstroms, about 1.7 angstroms, about 1.6 angstroms, or about 1.5 angstroms. Suitable methods and programs for forming a bond and analyzing bond length are well-known in the art, and include Discovery Studio® and Charmm (Accelrys, Inc. San Diego), Amber (Amber Software Administrator, USSF, 600 16th Street, Room 552, San Fransico, Calif. 94158 and http://ambermd.org/), Guassian (340 Quinnipiac St. Bldg 40, Wallingford Conn. 06292 USA and www.gaussian.com/), Qsite (Schrodinger Inc., New York), and covalent docking programs (BioSolvIT GmbH, Germany www.biosolveit.de), Maestro™, MacroModel™ and Jaguar™ (Modeling softwar packages, Schrodinger, LLC. 120 West 45th Street, New York, N.Y. 10036-4041).

If desired, the compounds designed using the method can be further analyzed and/or refined structurally. For example, if desired, the invention can include the further step of determining whether the binding site of the target polypeptide is blocked (i.e., ligand, substrate or cofactor is not able to bind to the binding site) when a covalent bond is formed between the sulfur atom of the Cys residue in the binding site and the reactive chemical functionality of the warhead. This step can be performed using a structural model of the target polypeptide—irreversible inhibitor that covalently binds a Cys residue complex. It is possible that the binding of the inhibitor to the target polypeptide will be altered upon formation of a covalent bond between the reactive chemical functionality and the Cys residue. However, in most cases, the compound will still block the binding site of the target polypeptide and prevent ligands, substrates or cofactors from binding to the binding site. Alterations in the binding mode of the inhibitor upon formation of a covalent bond, and whether the binding site remains blocked, can be determined by analysis of the structural model of the inhibitor complexed to the target polypeptide after covalent bond formation using suitable methods and programs disclosed herein.

In another example, compounds designed using the invention are further analyzed for favorable or preferred characteristics, such as the conformation of the covalent bond formed. As described in Examples 1 and 6, covalent bonds formed between a Cys and an acrylamide warhead can have a cis-conformation or trans-conformation of the amide, with the trans-conformation being preferred. In another example, preferred compounds are selected from compounds that have similar structures based on the energy of product formed by reaction of the warhead and the Cys residue, with lower energy products being preferred. The energy of the products can be determined using any suitable method, such as using quantum mechanics or molecular mechanics.

The invention can be used to design inhibitors that covalently bind any desired target polypeptide by forming a covalent bond with a Cys residue in a binding site of the target polypeptide. It is preferred that the Cys residue that forms a covalent bond with the inhibitor designed according to the invention is not conserved in the protein family that contains the target polypeptide. By virtue of the Cys residue not being conserved, it is possible to convert promiscuous reversible inhibitors which inhibit several members of a protein family into more selective irreversible inhibitors which inhibit fewer members or even a single member of the protein family.

In some applications of the invention, the target polypeptide has a catalytic activity. For example, the target polypeptide can be a kinase, a protease, such as a viral protease, a phosphatase, or other enzyme. When the target polypeptide has catalytic activity it is preferred that the Cys reside that forms a covalent bond with the inhibitor designed according to the invention is not a catalytic residue. In certain preferred embodiments, the irreversible inhibitor designed using the invention is not a suicide or mechanism-based inhibitor, which are inhibitors resulting from the process of an enzyme converting a substrate into a covalent inactivator during the catalytic process.

Preferably, the reversible inhibitor binds to a site on the target polypeptide that is a binding site for a ligand, cofactor or substrate. When the target polypeptide is a kinase, it is preferred that the reversible inhibitor binds to or interacts with the ATP-binding site of the kinase. For example, the reversible inhibitor can interact with the hinge region of the ATP binding site.

The algorithm and method described herein can be performed using the complete structure of the binding site of the target polypeptide and the structure of a reversible inhibitor. Optionally, the structure of the reversible inhibitor and only the Cys of the binding site of the target polypeptide is considered when the algorithm is performed. In this option, the three dimensional orientation of the Cys residue and the reversible inhibitor are the same as they are in the presence of the rest of the structure of the binding site of the target polypeptide. Once an irreversible inhibitor or candidate irreversible inhibitor is designed by considering only the Cys of the binding site, the full model of the binding site can be considered, if desired, to provide additional structural information and constraints that may identify steric clashes that reduces the number of substitutable positions that will result in the warhead being within bonding distance of a Cys in the binding site. In the examples described herein, the algorithm was performed considering the structure of the reversible inhibitor and the Cys of the binding site of the target polypeptide. This approach successfully produced irreversible inhibitors of several target polypeptides. The number of substitutable positions on the reversible inhibitors that were identified in the work described in the examples was small, so the additional constrains that might be imposed by the full model of the binding site were not needed, but could have been used.

For convenience, the steps of the algorithm and method are described herein in an order that allows for a clear and concise description of the invention. However, while it is preferred that the method steps are performed sequentially in the order described, they may be performed in any suitable order. For example, the method can be performed by forming a bond between a warhead and a Cys residue to form an adduct, and then bonding the warhead to a substitutable position on the reversible inhibitor, optionally through a linker.

### Enone-Containing Warheads, Irreversible Inhibitors and Conjugates

The invention also relates to irreversible inhibitors that have a warhead that contains a conjugated enone, an α, β unsaturated carbonyl. The invention also relates to polypeptide conjugates formed by the reaction of a conjugated enone warhead with the —SH of a Cysteine residue in a polypeptide. Enones are a class of reactive functionalities that contain the structure —C(O)—CH═CH—. This structure can be part of a linear, branched or cyclic chemical moiety. Enones provide the advantage that they are generally of low reactivity and do not react with the —SH of cysteine in solution. However, when positioned within bonding distance of a Cys in a polypeptide, the enone can selectively react with the —SH of the cysteine residue. Thus, conjugated enones can be used to provide highly selective warheads and irreversible inhibitors.

In one aspect, the warhead comprising a conjugated enone has the formula

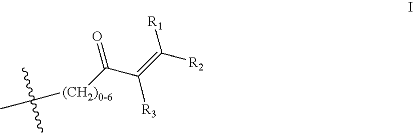

wherein R₁, R₂ and R₃ are independently hydrogen, C₁-C₆ alkyl, or C₁-C₆ alkyl that is substituted with —NRxRy; Rx and Ry are independently hydrogen or C₁-C₆ alkyl.

Exemplary warheads comprising a conjugated enone include I-a-I-h.

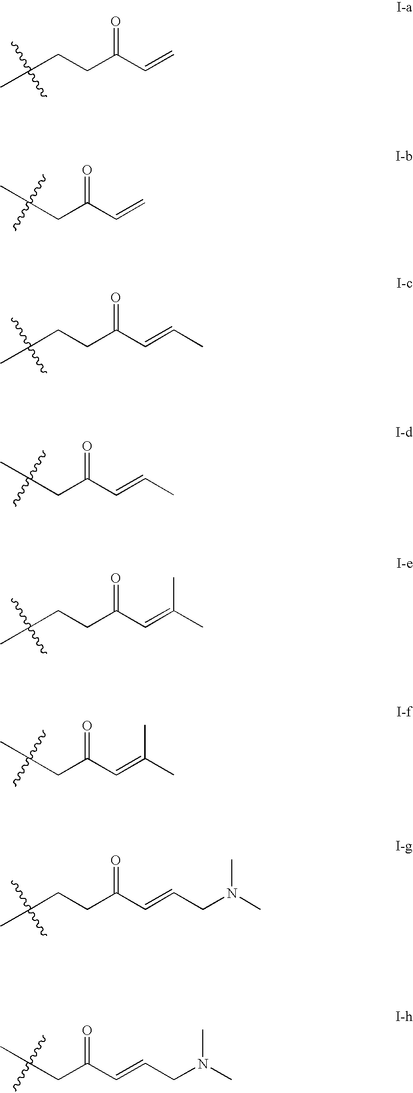

The invention relates to irreversible inhibitors that comprise a conjugated enone warhead that forms a covalent bond with cysteine residue of a target polypeptide, such as irreversible inhibitors designed using the algorithm of the invention. In some embodiments, the conjugated enone warhead is of formula I. In particular embodiments, the conjugated enone war head is selected from I-a, I-b, I-c, I-d, I-e, I-f and I-g.

The invention also relates to a method of irreversibly inhibiting a target polypeptide by contacting a polypeptide containing a binding site that has a cysteine residue with an irreversible inhibitor that comprises a conjugated enone warhead that forms a covalent bond with the cysteine residue of the target polypeptide, such as an irreversible inhibitor designed using the algorithm of the invention.

The invention also relates to polypeptide conjugates formed by the reaction of a conjugated enone-containing warhead with the —SH group of a Cys residue. Such conjugates have a variety of uses. For example, the amount of conjugated target polypeptide relative to unconjugated target polypeptide in a biological sample obtained from a patient that has been treated with an irreversible inhibitor that contains a conjugated enone warhead can be used to tailor dosing (e.g., quantity administered and/or time interval between administrations). In one aspect the conjugate has the formula

X-M-S—CH₂—R

wherein:

X is a chemical moiety that binds to the binding site of a target polypeptide, wherein the binding site contains a cysteine residue.

M is a modifier moiety formed by the covalent bonding of a conjugated enone-containing warhead group with the sulfur atom of said cysteine residue;

S—CH₂ is the side chain sulfur-methylene of said cysteine residue; and

R is the remainder of the target polypeptide.

In some embodiments, the conjugated enone-containing warhead is of formula I, and the conjugate is of formula II:

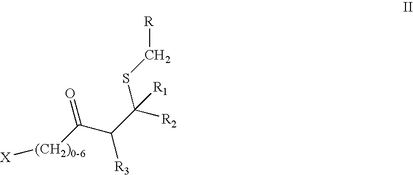

wherein X is a chemical moiety that binds to the binding site of a target polypeptide, wherein the binding site contains a cysteine residue;

S—CH₂ is the side chain of said cysteine residue;

R is the remainder of the target polypeptide;

R₁, R₂ and R₃ are independently hydrogen, C₁-C₆ alkyl, or C₁-C₆ alkyl that is substituted with —NRxRy; and Rx and Ry are independently hydrogen or C₁-C₆ alkyl.

In particular embodiments, the conjugate has a formula selected from II-a, II-b, II-c, II-d, II-e, II-f, II-g and II-h, wherein X and R are as defined in Formula II.

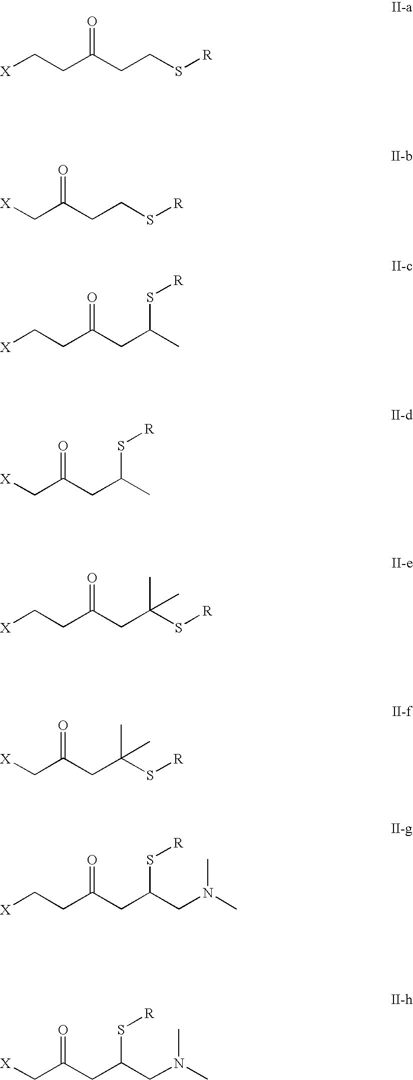

### EXEMPLIFICATION

### Example 1

### Irreversible Imatinib

Imatinib is a potent reversible inhibitor of cKIT, PDGFR, ABL, and CSF1R kinases. Using the design algorithm described herein, this reversible inhibitor was rapidly and efficiently converted into an irreversible inhibitor of cKit, PDGFR and CSF1R kinases. In addition, it is shown that the subject method identifies when it is not possible to readily convert a reversible inhibitor of a target into an irreversible inhibitor of that target, as was the case in the ensuing example for imatinib and the target ABL.

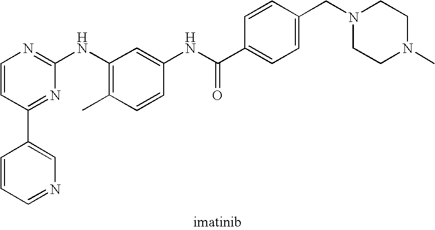

A. cKIT

### Design Method

The coordinates for the x-ray complex of cKIT bound to imatinib (pdbcode 1T46) were obtained from the protein databank (world wide web rcsb.org). The coordinates of imatinib were extracted and all protein Cys residues within 20 angstroms of imatinib when bound to cKIT were identified using Discovery Studio (v2.0.1.7347; Acccelrys Inc., CA). This identified seven residues Cys660, Cys673, Cys674, Cys788, Cys809, Cys884, and Cys906. Then 15 substitutable positions were explored in three-dimensions on the imatinib template (Formula I-1) to determine which could be substituted with a warhead so that the warhead would form a covalent bond with one of the Cys residues (Cys660, Cys673, Cys674, Cys788,Cys809, Cys884 or Cys906) in the cKIT binding site.

Design Method 1.1

In this method, warheads were manually built on the imatinib template and then molecular dynamics was used to assess the capabilities of the warheads to form bonds with the Cys in the binding site of cKit. Acrylamide warheads were built in three dimensions onto the imatinib template using Discovery Studio. The imatinib template is shown in Formula I-1. The structures of the resulting compounds were checked to determine the position of the warheads and to determine if the warheads could reach any of the identified Cys residues in the binding site.

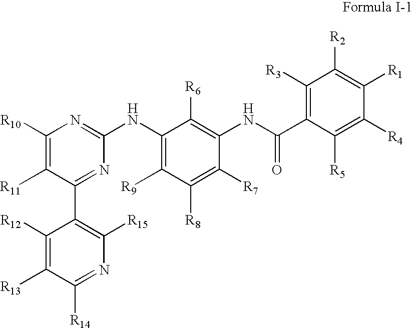

In order to sample the flexibility of the warheads and the side chain positions, a molecular dynamics simulation of the warheads and side chain positions was performed and analyzed to determine if the warhead was within 6 angstroms of any of the Cys residues in the binding site, and whether there were steric clashes between the warheads and the residues. Standard settings were used in the Standard Dynamics Cascade Simulations protocol of Discovery Studio for the molecular dynamics simulations. The MMFF forcefield in Discovery Studio with a 4 ps simulation was used. The coordinates of the non-warhead positions and the Cys main-chain atoms were held fixed during the molecular dynamics simulation.

This simulation identified three template positions which were near Cys788 and two near Cys809 of cKIT. (Table 5).

TABLE 5

| | |
|---|---|
| Distance to | |
| CYS788 or | Can Bond be Formed |

| Position | Placement | CYS809 | CYS788 | CYS809 |
|---|---|---|---|---|
| R₁ | | OK | YES | NO |
| R₂ | | OK | YES | NO |
| R₃ | STERIC | | | |
| | CLASH | | | |
| R₄ | | OK | YES | NO |
| R₅ | STERIC | | | |
| | CLASH | | | |
| R₆ | STERIC | | | |
| | CLASH | | | |
| R₇ | | TOO FAR | | |
| R₈ | | TOO FAR | | |
| R₉ | | TOO FAR | | |
| R₁₀ | | OK | NO | NO |
| R₁₁ | | OK | NO | NO |
| R₁₂ | STERIC | | | |
| | CLASH | | | |
| R₁₃ | | TOO FAR | | |
| R₁₄ | STERIC | | | |
| | CLASH | | | |
| R₁₅ | STERIC | | | |
| | CLASH | | | |

These five template positions were then subject to a final filter that required that the acrylamide reaction product could be formed between the candidate inhibitor and the Cys residue (Cys788 or Cys809), which involved forming a bond of less than 2 angstroms using a standard molecular dynamics simulation. This constraint left three template positions, R₁, R₂ and R₄, but only one Cys residue, Cys788. Of these template positions, the bonds that involved the warheads at positions R₂ and R₄ involved a cis-conformation of the amide group of the warhead, which is less preferred. The bond that involved the warhead at position R₁ involved a trans-conformation of the amide group of the warhead, which is preferred.

Design Method 1.2

In this method, warheads were automatically modeled on the imatinib template and then molecular docking was used to assess their bond forming ability with the Cys in the binding site of cKit.

The warheads were built on the imatinib template using the Accelryes SciTegic Pipeline enumeration protocol, which resulted in 13 virtual compounds from 15 possible virtual compounds. This was due to R₂ and R₄ as well as R₃ and R₅ (Formula I-1) being equivalent due to symmetry, and therefore only R₂ and R₃ were evaluated further. These compounds were then converted into 3D using the ligand preparation protocol in Discovery Studio. These 3D virtual compounds were then docked into the cKit xray structure using the CDOCKER protocol of Discovery Studio. A constrained docking algorithm was used in which the core of imatinib as defined in the xray structure (Formulae I-1) was used as a constraint in the docking procedure. Ten conformations of each virtual compound were produced and the top conformation of each compound was assessed for its proximity by distance to the Cys in the binding site of cKit. After applying the distance filter (<6 angstroms), only two of the compounds, with warheads at R₁ and R₂ were found to be close to a Cys in the binding site. These two compounds were both near to Cys788 and they were then assessed for bond forming capability. The protein and compounds were held fixed, but the side chain of Cys788 and the warhead were not constrained. After the minimization was completed, the newly formed covalent bond and the potential energy were examined. The virtual compound with the warhead at the R₁ position was ranked as the most preferred.

As detailed below, two compounds with an acrylamide at the R₁ position, Compound 1 and Compound 2, were synthesized and shown to inhibit cKit.

### Synthesis of Compounds

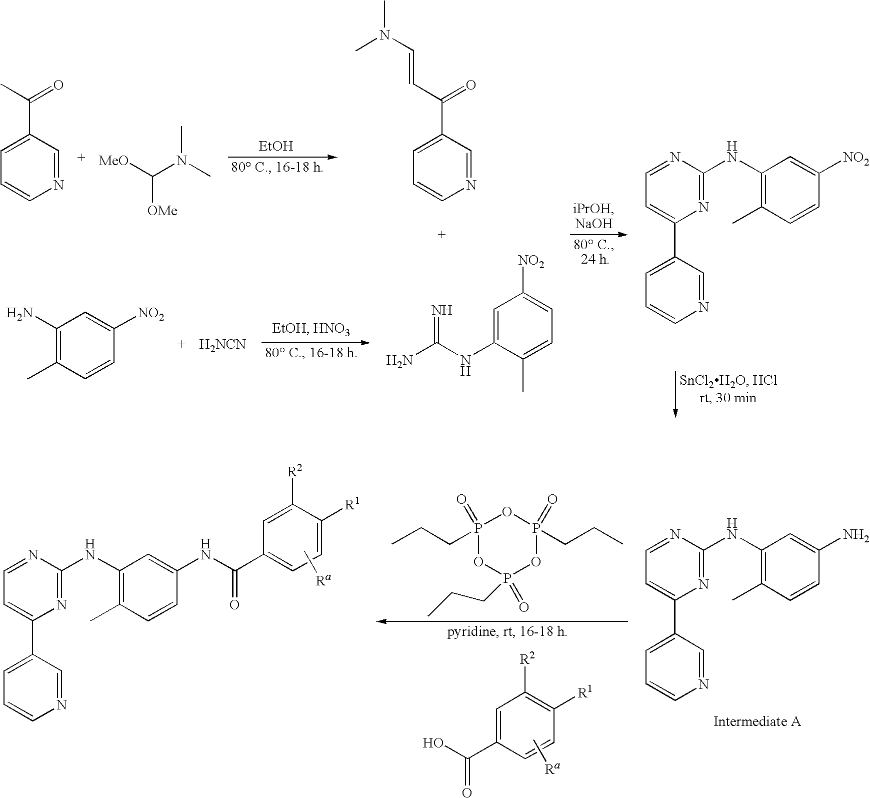

### Synthesis of Intermediate A

Step 1: 3-Dimethylamino-1-pyridin-3-yl-propenone: 3-Acetylpyridine (2.5 g, 20.64 mmol) and N,N-dimethyl-formamide dimethylacetal (3.20 ml, 24 mmol) were refluxed in ethanol (10 mL) overnight. The reaction mixture was cooled to room temperature and evaporated under reduced pressure. Diethyl ether (20 mL) was added to the residue and the mixture was cooled to 0° C. The mixture was filtered to give 3-dimethylamino-1-pyridin-3-yl-propenone (1.9 g, 10.78 mmol) as yellow crystals. (Yield: 52%.) This material was used in subsequent steps without further purification.

Step 2: N-(2-Methyl-5-nitro-phenyl)-guanidinium nitrate: 2-Methyl-5-nitro aniline (10 g, 65 mmol) was dissolved in ethanol (25 mL), and concentrated HNO₃ (4.6 mL) was added to the solution dropwise followed by 50% aqueous solution of cyanamide (99 mmol). The reaction mixture was refluxed overnight and then cooled to 0° C. The mixture was filtered and the residue was washed with ethyl acetate and diethyl ether and dried to provide N-(2-Methyl-5-nitro-phenyl)-guanidinium nitrate (4.25 g, yield: 34%).

Step 3: 2-methyl-5-nitrophenyl-(4-pyridin-3-yl-pyrimidin-2-yl)-amine: To a suspension of 3-dimethylamino-1-pyridin-3-yl-propenone (1.70 g, 9.6 mmol) and N-(2-methyl-5-nitro-phenyl)-guanidinium nitrate (2.47 g, 9.6 mmol) in 2-propanol (20 mL) was added NaOH (430 mg, 10.75 mmol) and the resulting mixture was refluxed for 24 h. The reaction mixture was cooled to 0° C. and the resulting precipitate was filtered. The solid residue was suspended in water and filtered and then washed with 2-propanol and diethyl ether and dried. 0.87 g (2.83 mmol) of 2-methyl-5-nitrophenyl-(4-pyridin-3-yl-pyrimidin-2-yl)-amine was isolated. (Yield: 30%.)

Step 4: 4-Methyl-N-3-(4-pyridin-3-yl-pyrimidin-2-yl)-benzene-1,3-diamine (Intermediate A): A solution of SnCl₂.2H₂O (2.14 g, 9.48 mmol) in concentrated hydrochloric acid (8 mL) was added to 2-methyl-5-nitro-phenyl-(4-pyridin-3-yl-pyrimidin-2-yl)-amine (0.61 g, 1.98 mmol) with vigorous stirring. After 30 min of stirring the mixture was poured onto crushed ice, made alkaline with K₂CO₃, and extracted three times with ethyl acetate (50 ml). Organic phases were combined, dried over MgSO₄, and evaporated to dryness. Recrystallization from dichloromethane resulted in 252.6 mg (0.91 mmol) of 4-methyl-N-3-(4-pyridin-3-yl-pyrimidin-2-yl)-benzene-1,3-diamine (yield: 46%) as an off-white solid.

### Synthesis of Compound 1

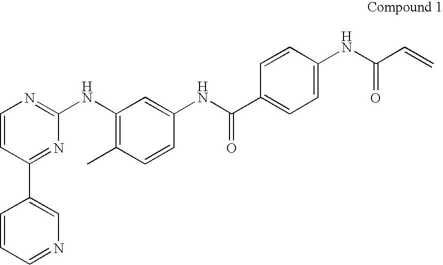

Step 1: 4-(acrylamido)benzoic acid A solution of 4-aminobenzoic acid (1.40 g, 10 mmol) in DMF (10 mL) and pyridine (0.5 ml) was cooled to 0° C. To this solution was added of acryloyl chloride (0.94 g, 10 mmol) and the resulting mixture was stirred for 3 hours. The mixture was poured into 200 ml of water and the white solid obtained was filtered, washed with water and ether. Drying under high vacuum provided 1.8 g of the desired product which was used in the next step without purification.

Step 2: 4-(Acrylamido)benzoic acid (82 mg, 0.43 mmol) and Intermediate A (100 mg, 0.36 mmol) were dissolved in pyridine (4 ml) under nitrogen and stirred. To this solution was added 1-propane phosphonic acid cyclic anhydride (0.28 g, 0.43 mmol) and the resulting solution was stirred overnight at room temperature. The solvent was evaporated to a small volume and then poured into a 50 ml of cold water. The solid formed was filtered and a yellow powder was obtained. Purification of the crude product by column chromatography (95:5 CHCl₃: MeOH) provided 30 mg of 4-acrylamido-N-(4-methyl-3-(4-(pyridin-3-yl)pyrimidin-2-ylamino)phenyl)benzamide (Compound 1) as a white powder. MS (M+H+): 251.2; ¹H NMR (DMSO-D₆, 300 MHz) δ (ppm): 10.42 (s, 1H), 10.11 (s, 1H), 9.26 (d, 1H, J=2.2 Hz), 8.99 (s, 1H), 8.68 (dd, 1H, J=3.0 and 1.7 Hz), 8.51 (d, 1H, J=5.2 Hz), 8.48 (m, 1H), 8.07 (d, 1H, J=1.7 Hz), 7.95 (d, 2H, J=8.8 Hz), 7.79 (d, 2H, J=8.8 Hz), 7.45 (m, 3H), 7.19 (d, 1H, J=8.5 Hz), 6.47 (dd, 1H, J=16.7 and 9.6 Hz), 6.30 (dd, H, J=16.7 and 1.9 Hz), 5.81 (dd, 1H, J=9.9 and 2.2 Hz), 2.22 (s, 3H).

### Synthesis of Compound 2

### 4-Acrylamido-N-(4-methyl-3-(4-pyridin-3-yl)pyrimidin-2-ylamino)phenyl-3-(trifluoromethyl)benzamide

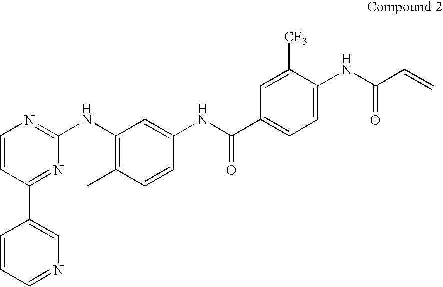

### 1) Methyl 4-acrylamido-3-nitrobenzoate

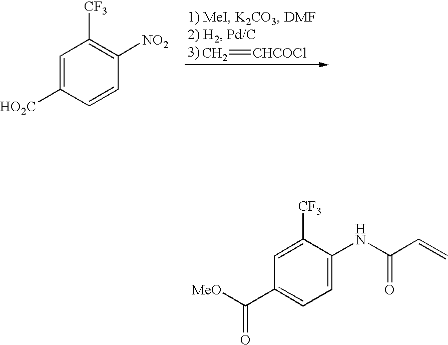

Methyl iodide (1.4 g, 9.86 mmol) was added dropwise to a stirred solution of 4-nitro-3-(trifluoromethyl)benzoic acid (1.0 g, 4.25 mmol) and potassium carbonate (1.5 g, 10.85 mmol) in 30 mL DMF at room temperature. The mixture was stirred at rt overnight. Diethyl ether (120 mL) was added and the mixture was washed with water, was dried over Na₂SO₄, was filtered and was concentrated under reduced pressure to give 1.0 g of crude methyl 4-nitro-3-(trifluoromethyl)benzoate. A solution of 0.87 g (3.49 mmol) of methyl 4-nitro-3-(trifluoromethyl)benzoate and 0.2 g 10% Pd/C in 30 mL methanol was stirred under hydrogen (40 psi) at rt overnight. The mixture was filtered and was concentrated under reduced pressure to give 0.8 g of crude methyl 4-amino-3-(trifluoromethyl)benzoate as a white solid. Acryloyl chloride (0.35 mL, 3.65 mmol) was added to a solution of 0.8 g of methyl 4-amino-3-(trifluoromethyl)benzoate and triethylamine (0.9 g, 8.9 mmol) in 40 mL of dichloromethane at 0° C. After stirring for 3 hours at rt this solution was washed with successively saturated aqueous NaHCO₃, and saturated aqueous NaCl. The dichloromethane solution was dried over Na₂SO₄ and was concentrated in vacuo to give a crude product, which was further purified by chromatography over silica gel using 1% CH₃OH—CH₂Cl₂ to give 0.818 mg of the title compound as a white solid.

### 2) 4-Acrylamido-3-nitrobenzoic acid

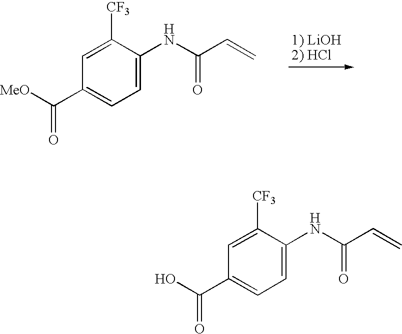

To a stirred solution of methyl 4-acrylamido-3-nitrobenzoate (0.8 g, 2.93 mmol) in 20 mL THF was added 20 mL of 1 N LiOH solution. The resulting solution was acidified to pH 1 with 10% aqueous HCl and was then extracted with three 40-mL portions of ethyl acetate. The combined ethyl acetate extract was washed with saturated aqueous NaCl, was dried over Na₂SO₄, was filtered and was concentrated to dyness in vacuo to give 0.75 g of the title compound as a white solid.

### 3) 4-Acrylamido-N-(4-methyl-3-(4-pyridin-3-yl)pyrimidin-2-ylamino)phenyl-3-(trifluoromethyl)benzamide

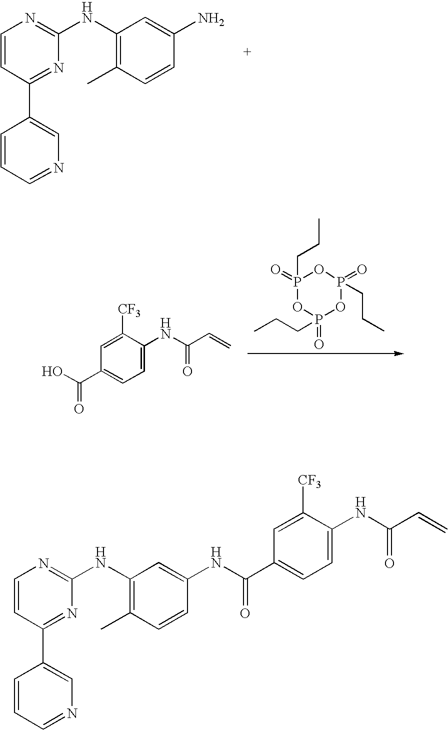

To a stirred solution of N-(4-methyl-3-(4-pyridin-3-yl)pyrimidin-2-ylamino)phenylamine (87 mg, 0.31 mmol) and 4-acrylamido-3-(trifluoromethyl)benzoic acid (95 mg, 0.37 mmol) in 10 mL pyridine was added 250 mg (0.39 mmol) propylphosphonic anhydride. The resulting solution was stirred at rt for 72 hr. The solvent was removed in vacuo and the residue was stirred with 50 mL water to give a yellow solid that was isolated by filtration. Purification of the crude product by silica gel chromatography using 5% CH₃OH—CH₂Cl₂ gave 101 mg of the title compound. ¹H NMR (DMSO-d₆, 300 MHz) δ (ppm): 10.41 (s, 1H), 9.90 (s, 1H), 9.28 (d, 1H), 8.98 (s, 1H), 8.69 (d, 1H), 8.68 (dd, 1H), 8.49 (m, 1H), 8.29 (s, 1H), 8.24 (d, 1H), 8.08 (d, 1H), 7.79, (m, 3H), 7.23 (d, 1H), 6.59 (dd, 1H), 6.28 (dd, 1H), 5.81 (dd, 1H), 2.24 (s, 3H).

cKIT Inhibition Assay

Compounds were assayed as inhibitors of c-KIT using human recombinant c-KIT (obtained from Millipore, catalog number 14-559) and monitoring the phosphorylation of a fluorescein-labeled peptide substrate (1.5 μM). Reactions were carried out in 100 mM HEPES (pH 7.5), 10 mM MnCl₂, 1 mM DDT, 0.015% Brij-35, and 300 μM ATP, with and without test compound. The reaction was started by adding the ATP and incubating for 1 hour at room temperature. The reaction was terminated by the addition of stop buffer containing 100 mM HEPES (pH 7.5), 30 mM EDTA, 0.015% Brij-35, and 5% DMSO. Phosphorylated and unphosphorylated substrate was separated by charge using electrophoretic mobility shift. Product formed was compared to control wells to determine inhibition or enhancement of enzyme activity. c-KIT inhibition data for Compound 1 and Compound 2 is provided in Table 6.

TABLE 6
c-KIT Inhibition Data

| Compound # | % Inhibition | Concentration (μM) |
|---|---|---|
| 1 | 82 | 1 |
| 2 | 88 | 1 |

B. PDGFR

Design Method

Using the coordinates for the x-ray complex of cKIT bound to imatinib (pdbcode 1T46) as described above, a homology model of PDGFR-alpha kinase (Uniprot code: P16234) was generated. The homology model was built using the Build Homology module in Discovery Studio using the cKIT-PDGFRα alignment shown. Then the 15 substitutable positions on the imatinib template were explored in three-dimensions to determine which could be substituted with a warhead so that the warhead would form a covalent bond with the Cys in the binding site. The methodology identified three template positions, R₁, R₂, and R₄, and Cys814 capable of forming a covalent bond with an acrylamide warhead. Of these template positions, the bonds that involved the warheads at positions R₂ and R₄ involved a cis-conformation of the amide group of the warhead, which is less preferred. The bond that involved the warhead at position R₁ involved a trans-conformation of the amide group of the warhead, which is preferred.

| | |
|---|---|
| CKIT | GNNYVYIDPTQLPYDHKWEFPRNRLSFGKTLGAGAFGKVVEATAYGLIKSDAAMTVAVKMLKP |
| PDGFRALPHA | GHEYIYVDPMQLPYDSRWEFPRDGLVLGRVLGSGAFGKVVEGTAYGLSRSQPVMKVAVKMLKP |
| CKIT | SAHLTEREALMSELKVLSYLGNHMNIVNLLGACTIGGPTLVITEYCCYGDLLNFLRRKR---- |
| PDGFRALPHA | TARSSEKQALMSELKIMTHLGPHLNIVNLLGACTKSGPIYIITEYCFYGDLVNYLHKNRDSFL |
| CKIT | ---------------------------DSFLALDLEDLLSFSYQVAKGMAFLASKNCIHRDLA |
| PDGFRALPHA | QRSLYDRPASYKKKSMLDSEVKNLLSDDNSEGLTLLDLLSFTYQVARGMEFLASKNCVHRDLA |
| CKIT | ARNILLTHGRITKICDFGLARDIKNDSNYVVKGNARLPVKWMAPESIFNCVYTFESDVWSYGI |
| PDGFRALPHA | ARNVLLAQGKIVKICDFGLARDIMHDSNYVSKGSTFLPVKWMAPESIFDNLYTTLSDVWSYGI |
| CKIT | FLWELFSLGSSPYPGMPVDSKFYKMIKEGFRMLSPEHAPAEMYDIMKTCWDADPLKRPTFKQI |
| PDGFRALPHA | LLWEIFSLGGTPYPGMMVDSTFYNKIKSGYRMAKPDHATSEVYEIMVKCWNSEPEKRPSFYHL |
| CKIT | VQLIEKQISESTN |
| PDGFRALPHA | SEIVE-------- |

CKIT: human CKIT (SEQ ID NO: 1)
PDGFRALPHA: human PDGF Receptor Alpha (SEQ ID NO: 2)

### PDGFR Inhibition Assay

### Method A:

Compounds were assayed as inhibitors of PDGFR in a manner substantially similar to the method described by Invitrogen Corp (Invitrogen Corporation, 1600 Faraday Avenue, Carlsbad, Calif., CA; world wide web invitrogen.com/downloads/Z-LYTE_Brochure_{—}1205.pdf) using the Z′-LYTE™ biochemical assay procedure or similar biochemical assay. The Z′-LYTE™ biochemical assay employs a fluorescence-based, coupled-enzyme format and is based on the differential sensitivity of phosphorylated and non-phosphorylated peptides to proteolytic cleavage.

Compound 1 was tested at 0.1 μM and 1 μM in duplicate. Compound 1 showed a mean inhibition of PDGFR-α of 76% at 1 μM and 29% at 0.1 μM.

### Method B

Briefly, 10× stock of PDGFRα (PV3811) enzyme, 1.13×ATP (AS001A) and Y12-Sox peptide substrates (KCZ1001) was prepared in 1× kinase reaction buffer consisting of 20 mM Tris, pH 7.5, 5 mM MgCl₂, 1 mM EGTA, 5 mM β-glycerophosphate, 5% glycerol (10× stock, KBOO2A) and 0.2 mM DTT (DS001A). 5 μL of enzyme were pre-incubated in a Corning (#3574) 384-well, white, non-binding surface microtiter plate (Corning, N.Y.) for 30 min at 27° C. with a 0.5 volume of 50% DMSO and serially diluted compounds prepared in 50% DMSO. Kinase reactions were started with the addition of 45 μL of the ATP/Y9 or Y12-Sox peptide substrate mix and monitored every 30-9 seconds for 60 minutes at λₑₓ360/λₑₘ485 in a Synergy⁴ plate reader from BioTek (Winooski, Vt.). At the conclusion of each assay, progress curves from each well were examined for linear reaction kinetics and fit statistics (R², 95% confidence interval, absolute sum of squares). Initial velocity (0 minutes to 20+ minutes) from each reaction was determined from the slope of a plot of relative fluorescence units vs time (minutes) and then plotted against inhibitor concentration to estimate IC₅₀ from log [Inhibitor] vs Response, Variable Slope model in GraphPad Prism from GraphPad Software (San Diego, Calif.). [PDGFRα]=2-5 nM, [ATP]=60 μM and [Y9-Sox peptide]=10 μM (ATP K_{Mapp}=61 μM)

PDGFR inhibition data for Compound 1 and Compound 2 are set forth in Table 7.

TABLE 7
PDGFR Inhibition Data

| Compound # | % Inhibition | Conc. (μM) | IC₅₀ (nM) |
|---|---|---|---|
| 1 | 76 | 1 | 172.94 |
| 2 | 91 | 1 | 2.2 |

### PDGFR Mass Spectral Analysis of Compound 1

Mass spectral analysis of PDGFR-α in the presence of Compound 1 was performed. PDGFR-α protein (supplied from Invitrogen: PV3811) was incubated with 1 μM, 10 μM, and 100 μM Compound 1 for 60 minutes. Specifically, 1 μL of 0.4 μg/μL PDGFR-α (Invitrogen PV3811) stock solution (50 mM Tris HCl ph 7.5, 150 mM NaCl, 0.5 mM EDTA, 0.02% Triton X-100, 2 mM DTT, 50% glycerol) was added to 9 μL of Compound 1 in 10% DMSO (final concentration of 1 μM, 10 μM, and 100 μM). After 60 minutes, 9 μL of 50 mM ammonium bicarbonate, 3.3 μL of 6 mM iodoacetamide in 50 mM ammonium bicarbonate, and 1 μL of 35 ng/μL trypsin was added to stop the reaction.

The tryptic digest was analyzed by mass spectrometer (MALDI-TOF) at 10 μM. Of the five cysteine residues found in the PDGFR-α protein, four of the cysteine residues were identified as being modified by iodoacetamide, while the fifth cysteine residue was modified by the compound 1. Mass spectral analysis of the tryptic digests was consistent with Compound 1 being covalently bound to PDGFR-α protein at Cys814. MS/MS analysis of the tryptic digests confirmed presence of the Compound 1 at Cys814.

### EOL-1 Cellular Proliferation Assay

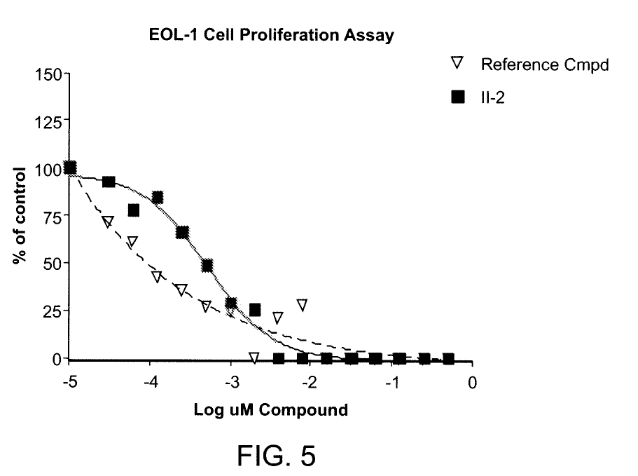

EOL-1 cells purchased from DSMZ (ACC 386) were maintained in RPMI (Invitrogen #21870)+10% FBS+1% penicillin/streptomycin (Invitrogen # 15140-122). For cell proliferation assays, cells in complete media were plated in 96 well plates at a density of 2×10⁴ cells/well and incubated in duplicate with compound ranging from 500 nM to 10 pM for 72 hours. Cell proliferation was assayed by measuring metabolic activity with Alamar Blue reagent (Invitrogen cat #DAL1100). After 8 hours incubation with Alamar Blue at 37° C., absorbance was read at 590 nm and the IC₅₀ of cellular proliferation was calculated using GraphPad. Dose response inhibition of cell proliferation of EOL-1 cells with reference compound and Compound 2 is depicted in FIG. 5.

### EOL-1 Cell Washout Assay

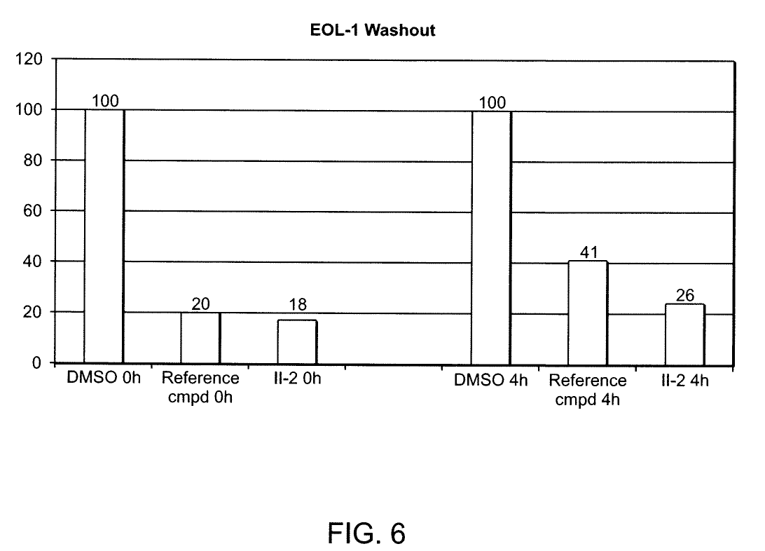

EOL-1 cells were grown in suspension in complete media and compound was added to 2×10⁶ cells per sample for 1 hour. After 1 hour, the cells were pelleted, the media was removed and replaced with compound-free media. Cells were washed every 2 hours and resuspended in fresh compound-free media. Cells were collected at specified timepoints, lysed in Cell Extraction Buffer and 15 μg total protein lysate was loaded in each lane. PDGFR phosphorylation was assay by western blot with Santa Cruz antibody sc-12910. The results of this experiment are depicted in FIG. 6 where it is shown that relative to DMSO control and to a reversible reference compound, Compound 2 maintained enzyme inhibition of PDGFR in EOL-1 cells after “washout” after 0 hours and 4 hours.

C. CSF1R

Design Method

Using the coordinates for the x-ray complex of cKIT bound to imatinib (pdbcode 1T46) as described above, a homology model of CSF1R kinase (Uniprot code: P07333) was generated. The homology model was built using the Build Homology module in Discovery Studio using the cKIT-CSF1R alignment shown. Then 15 substitutable positions on the imatinib template were explored in three-dimensions, to determine which could be substituted with a warhead so that the warhead would form a covalent bond with the Cys in the binding site. The methodology identified two template positions (R₁ and R₂) and Cys774 that could form a bond with an acrylamide warhead.

| | |
|---|---|
| CKIT | GNNYVYIDPTQLPYDHKWEFPRNRLSFGKTLGAGAFGKVVEATAYGLIKSDAAMTVAVKMLKP |
| CSF1R | GNSYTFIDPTQLPYNEKWEFPRNNLQFGKTLGAGAFGKVVEATAFGLGKEDAVLKVAVKMLKS |
| CKIT | SAHLTEREALMSELKVLSYLGNHMNIVNLLGACTIGGPTLVITEYCCYGDLLNFLRRKR---- |
| CSF1R | TAHADEKEALMSELKIMSHLGQHENIVNLLGACTHGGPVLVITEYCCYGDLLNFLRRKRPPGL |
| CKIT | --------DSFLALDLEDLLSFSYQVAKGMAFLASKNCIHRDLAARNILLTHGRITKICDFGL |
| CSF1R | EYSYNPSHNPEEQLSSRDLLHFSSQVAQGMAFLASKNCIHRDVAARNVLLTNGHVAKIGDFGL |
| CKIT | ARDIKNDSNYVVKGNARLPVKWMAPESIFNCVYTFESDVWSYGIFLWELFSLGSSPYPGMPVD |
| CSF1R | ARDIMNDSNYIVKGNARLPVKWMAPESIFDCVYTVQSDVWSYGILLWEIFSLGLNPYPGILVN |
| CKIT | SKFYKMIKEGFRMLSPEHAPAEMYDIMKTCWDADPLKRPTFKQIVQLIEKQISESTN |
| CSF1R | SKFYKLVKDGYQMAQPAFAPKNIYSIMQACWALEPTHRPTFQQICSFLQEQAQEDRR |

CKIT: human CKIT (SEQ ID NO: 1)
CSF1R: human SCF1R (SEQ ID NO: 3)

### CSF1R Inhibition Assay

Compounds were assayed as inhibitors of PDGFR in a manner substantially similar to the method described by Invitrogen Corp (Invitrogen Corporation, 1600 Faraday Avenue, Carlsbad, Calif., CA) using the Z′-LYTET™ biochemical assay procedure or similar biochemical assay. The 2×CSF1R (FMS)/Tyr 01 Peptide Mixture was prepared in 50 mM HEPES pH 7.5, 0.01% BRIJ-35, 10 mM MgCl₂, 1 mM EGTA. The final 104 Kinase Reaction consisted of 0.2-67.3 ng CSF1R (FMS) and 2 μM Tyr 01 Peptide in 50 mM HEPES pH 7.5, 0.01% BRIJ-35, 10 mM MgCl₂, 1 mM EGTA. After the 1 hour Kinase Reaction incubation, 5 μL of a 1:128 dilution of Development Reagent B was added.

Compound 1 showed 72% inhibition against CSF at 10 μM and Compound 2 showed 89% inhibition against CSF1R at 10 μM.

### Mass Spectral Analysis Data

Mass spectral analysis was used to determine whether Compound 2 was a covalent modifier of CSF1R. CSF1R (0.09 μg/μl) was incubated with Compound 2 (Mw 518.17) for 3 hrs at 10× excess prior to tryptic digestion. Iodoacetamide was used as the alkylating agent after compound incubation. For tryptic digests, a 2 μl aliquot (0.09 μg/μl) was diluted with 10 μl of 0.1% TFA prior to micro C18 Zip Tipping directly onto the MALDI target using alpha cyano-4-hydroxy cinnamic acid as the matrix (5 mg/ml in 0.1% TFA:Acetonitrile 50:50).

For tryptic digests, the instrument was set in Reflectron mode with a pulsed extraction setting of 1800. Calibration was done using the Laser Biolabs Pep Mix standard (1046.54, 1296.69, 1672.92, 2093.09, 2465.20). For CID/PSD analysis the peptide was selected using cursors to set ion gate timing and fragmentation occurred at a laser power about 20% higher and He was used as the collision gas for CID. Calibration for fragments was done using the P14R fragmentation calibration for the Curved field Reflectron. Database searching of the tryptic digest of CSF identified it correctly. Incorporating the Compound 2 modification (518.17) also identified the target peptide expected NCIHR (MH+642.31+518.17=1160.48) as the only modified peptide present. PSD analysis of this peptide signal (1160.50) gave enough fragments to allow for a database MS/MS ion search which verified the sequence of this peptide.

D. ABL

Design Method

Using the coordinates for the x-ray complex of cKIT bound to imatinib (pdbcode 1T46) as described above, a homology model of ABL kinase (Uniprot code: P00519) was generated. The homology model was built using the Build Homology module in Discovery Studio using the cKIT-ABL alignment shown. Then in three-dimensions, the 15 substitutable positions on the imatinib template were explored to place an acrylamide warhead to form a covalent bond with the Cys in the binding site. The methodology identified no template positions or a suitable Cys that could be modified.

| | |
|---|---|
| CKIT | GNNYVYIDPTQLPYDHKWEFPRNRLSFGKTLGAGAFGKVVEATAYGLIKSDAAMTVAVKMLKP |
| ABL | ----GAMDPSSPNYD-KWEMERTDITMKHKLGGGQYGEVYEG-----VWKKYSLTVAVKTLKE |
| CKIT | SAHLTEREALMSELKVLSYLGNHMNIVNLLGACTIGGPTLVITEYCCYGDLLNFLRRKRDSFL |
| ABL | DT--MEVEEFLKEAAVMKEIK-HPNLVQLLGVCTREPPFYIITEFMTYGNLLDYLRECNR--Q |
| CKIT | ALDLEDLLSFSYQVAKGMAFLASKNCIHRDLAARNILLTHGRITKICDFGLARDIKNDSNYVV |
| ABL | EVSAVVLLYMATQISSAMEYLEKKNFIHRDLAARNCLVGENHLVKVADFGLSRLMTGDT-YTA |
| CKIT | KGNARLPVKWMAPESIFNCVYTFESDVWSYGIFLWELFSLGSSPYPGMPVDSKFYKMIKEGFR |
| ABL | HAGAKFPIKWTAPESLAYNKFSIKSDVWAFGVLLWEIATYGMSPYPGIDLS-QVYELLEKDYR |
| CKIT | MLSPEHAPAEMYDIMKTCWDADPLKRPTFKQIVQLIEKQISESTN------------- |
| ABL | MERPEGCPEKVYELMRACWQWNPSDRPSFAEIHQAFETMFQESSISDEVEKELGKRGT |

CKIT: human CKIT (SEQ ID NO: 1)
ABL: human ABL (SEQ ID NO: 4)

### Example 2

### Irreversible Nilotinib

Nilotinib is a potent reversible inhibitor of ABL, cKIT, PDGFR and CSF1R kinase. Using the structure-based design algorithm described herein, nilotinib was rapidly and efficiently converted into an irreversible inhibitor that was shown to inhibit cKIT and PDGFR.

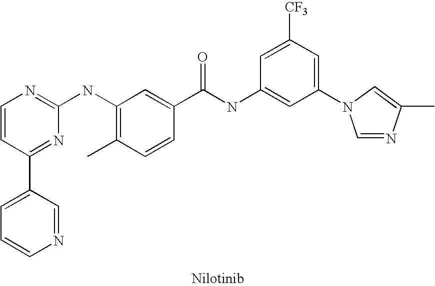

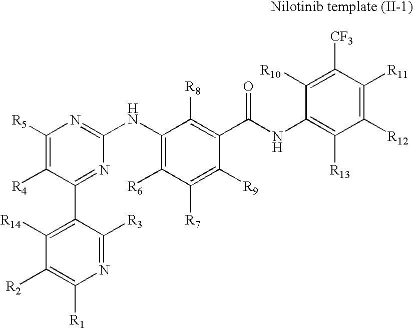

### A. ABL

The coordinates for the x-ray complex of nilotinib bound to Abl (pdbcode 3CS9) was obtained from the protein databank (world wide web rcsb.org). The coordinates of nilotinb were extracted and all protein Cys residues within 20 angstroms of nilotinib when bound to ABL were identified. Then, 14 substitutable positions on the nilotinib template (II-1) were explored in three-dimensions to determine which could be substituted with a chloroacetamide warhead to form a covalent bond with the Cys in the binding site. The methodology identified no template positions or a suitable Cys that could be modified

### B. PDGFRa

A homology model of PDGFR alpha kinase (Uniprot code: P16234) was produced using the x-ray structure of nilotinib bound to ABL as a template (pdbcode 3CS9). The homology model was built using the Build Homology module in Discovery Studio using the ABL-PDGFRa alignment shown. Then, 14 substitutable positions on the nilotinib template (II-1) were explored in three-dimensions to determine which could be substituted with a warhead to form a covalent bond with the Cys in the binding site. The methodology identified one template position (R₁₁) and one Cys (Cys814) capable of forming a covalent bond with a chloroacetamide warhead. Compound 3, which contains a chloroacetamide at R₁₁, was synthesized.

| | |
|---|---|
| PDGFRALPHA | GHEYIYVDPMQLPYDSRWEFPRDGLVLGRVLGSGAFGKVVEGTAYGLSRSQPVMKVAVKMLKP |
| ABL | ----GAMDPSSPNYD-KWEMERTDITMKHKLGGGQYGEVYEGVWKKYS-----LTVAVKTLKE |
| PDGFRALPHA | TARSSEKQALMSELKIMTHLGPHLNIVNLLGACTKSGPIYIITEYCFYGDLVNYLHKNRDSFL |
| ABL | DT--MEVEEFLKEAAVMKEIK-HPNLVQLLGVCTREPPFYIITEFMTYGNLLDYLRECN---- |
| PDGFRALPHA | SHHPEKPKKELDIFGLNPADESTRSYVILSFENNGDYMDMKQADTTQYVPMLERKEVSKYSDI |
| ABL | --------------------------------------------------------------- |
| PDGFRALPHA | QRSLYDRPASYKKKSMLDSEVKNLLSDDNSEGLTLLDLLSFTYQVARGMEFLASKNCVHRDLA |
| ABL | -----------------------------RQEVSAVVLLYMATQISSAMEYLEKKNFIHRDLA |
| PDGFRALPHA | ARNVLLAQGKIVKICDFGLARDIMHDSNYVSKGSTFLPVKWMAPESIFDNLYTTLSDVWSYGI |
| ABL | ARNCLVGENHLVKVADFGLSRLMTGDT-YTAHAGAKFPIKWTAPESLAYNKFSIKSDVWAFGV |
| PDGFRALPHA | LLWEIFSLGGTPYPGMMVDSTFYNKIKSGYRMAKPDHATSEVYEIMVKCWNSEPEKRPSFYHL |
| ABL | LLWEIATYGMSPYPGIDLS-QVYELLEKDYRMERPEGCPEKVYELMRACWQWNPSDRPSFAEI |
| PDGFRALPHA | SEIVE--------------------- |
| ABL | HQAFETMFQESSISDEVEKELGKRGT |

PDGFRALPHA: human PDGF Receptor Alpha (SEQ ID NO: 2)
ABL: human ABL (SEQ ID NO: 4)

### C. CSF1R

A homology model of CSF1R kinase (Uniprot code: P07333) was produced using the x-ray structure of nilotinib bound to ABL as a template (pdbcode 3CS9). The homology model was built using the Build Homology module in Discovery Studio using the ABL-CSF1R alignment shown. Then, 14 substitutable positions on the nilotinib template (II-1) were explored in three-dimensions to determine which could be substituted with a warhead to form a covalent bond with the Cys in the binding site. The methodology identified one template position (R₁₁) and one Cys (Cys774) that could form a bond with a chloroacetamide warhead.

| | |
|---|---|
| CSF1R | GNSYTFIDPTQLPYNEKWEFPRNNLQFGKTLGAGAFGKVVEATAFGLGKEDAVLKVAVKMLKS |
| ABL | ----GAMDPSSPNYD-KWEMERTDITMKHKLGGGQYGEVYEGVWKKYS-----LTVAVKTLKE |
| CSF1R | TAHADEKEALMSELKIMSHLGQHENIVNLLGACTHGGPVLVITEYCCYGDLLNFLRRKRP--- |
| ABL | DT--MEVEEFLKEAAVMKEIK-HPNLVQLLGVCTREPPFYIITEFMTYGNLLDYLRECN---- |
| CSF1R | -------------------------------------------------PGLEY--------- |
| ABL | --------------------------------------------------------------- |
| CSF1R | ---------SYNP------------SHNPEEQLSSRDLLHFSSQVAQGMAFLASKNCIHRDVA |
| ABL | -----------------------------RQEVSAVVLLYMATQISSAMEYLEKKNFIHRDLA |
| CSF1R | ARNVLLTNGHVAKIGDFGLARDIMNDSNYIVKGNARLPVKWMAPESIFDCVYTVQSDVWSYGI |
| ABL | ARNCLVGENHLVKVADFGLSRLMTGDT-YTAHAGAKFPIKWTAPESLAYNKFSIKSDVWAFGV |
| CSF1R | LLWEIFSLGLNPYPGILVNSKFYKLVKDGYQMAQPAFAPKNIYSIMQACWALEPTHRPTFQQI |
| ABL | LLWEIATYGMSPYPGIDLS-QVYELLEKDYRMERPEGCPEKVYELMRACWQWNPSDRPSFAEI |
| CSF1R | CSFLQEQAQEDRR------------- |
| ABL | HQAFETMFQESSISDEVEKELGKRGT |

CSF1R: human CSF1R (SEQ ID NO: 3)
ABL: human ABL (SEQ ID NO: 4)

D. cKIT

A homology model of cKIT kinase (Uniprot code: P10721) was produced using the x-ray structure of nilotinib bound to ABL as a template (pdbcode 3CS9). The homology model was built using the Build Homology module in Discovery Studio using the ABL-cKIT alignment shown. Then, 14 substitutable positions on the nilotinib template (II-1) were explored in three-dimensions to determine which could be substituted with a chloroacetamide warhead to form a covalent bond with the Cys in the binding site. This constraint left one template position (R₁₁) and one Cys (Cys788).

| | |
|---|---|
| CKIT | GNNYVYIDPTQLPYDHKWEFPRNRLSFGKTLGAGAFGKVVEATAYGLIKSDAAMTVAVKMLKP |
| ABL | ----GAMDPSSPNYD-KWEMERTDITMKHKLGGGQYGEVYEG-----VWKKYSLTVAVKTLKE |
| CKIT | SAHLTEREALMSELKVLSYLGNHMNIVNLLGACTIGGPTLVITEYCCYGDLLNFLRRKRDSFL |
| ABL | DT--MEVEEFLKEAAVMKEIK-HPNLVQLLGVCTREPPFYIITEFMTYGNLLDYLRECNR--Q |
| CKIT | ALDLEDLLSFSYQVAKGMAFLASKNCIHRDLAARNILLTHGRITKICDFGLARDIKNDSNYVV |
| ABL | EVSAVVLLYMATQISSAMEYLEKKNFIHRDLAARNCLVGENHLVKVADFGLSRLMTGDT-YTA |
| CKIT | KGNARLPVKWMAPESIFNCVYTFESDVWSYGIFLWELFSLGSSPYPGMPVDSKFYKMIKEGFR |
| ABL | HAGAKFPIKWTAPESLAYNKFSIKSDVWAFGVLLWEIATYGMSPYPGIDLS-QVYELLEKDYR |
| CKIT | MLSPEHAPAEMYDIMKTCWDADPLKRPTFKQIVQLIEKQISESTN------------- |
| ABL | MERPEGCPEKVYELMRACWQWNPSDRPSFAEIHQAFETMFQESSISDEVEKELGKRGT |

CKIT: human CKIT (SEQ ID NO: 1)
ABL: human ABL (SEQ ID NO: 4)

### Synthesis of Compound 3

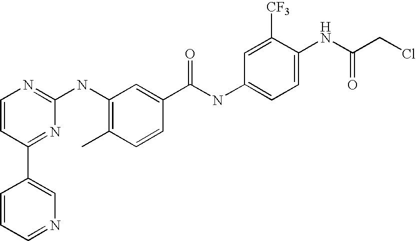

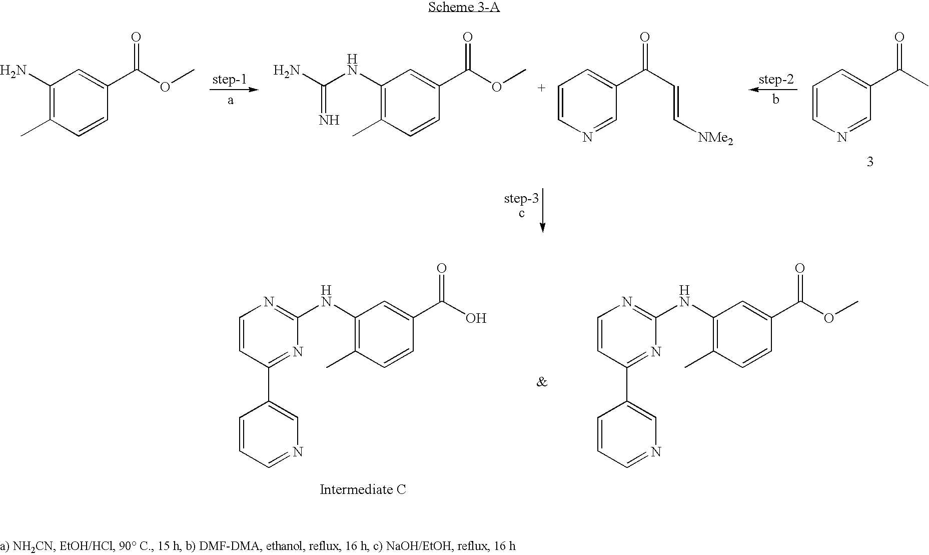

Step-1: To a stirred solution of the aniline ester (5 g, 30.27 mmol) in ethanol (12.5 mL) was added conc. HNO₃ (3 mL), followed by 50% aq. solution of cyanamide (1.9 g, 46.0 mmol) at rt. The reaction mixture was heated at 90° C. for 16 h and then cooled to 0° C. A solid precipitated out which was filtered, washed with ethyl acetate (10 mL), diethyl ether (10 mL), and dried to give the corresponding guanidine (4.8 g, 76.5%) as a light pink solid which was used without further purifications.
Step-2: A stirred solution of 3-acetyl pyridine (10.0 g, 82.56 mmol) and N,N-dimethylformamide dimethyl acetal (12.8 g, 96.00 mmol) in ethanol (40 mL) was refluxed for 16 h. It was then cooled to rt and concentrated under reduced pressure to get a crude mass. The residue was taken in ether (10 mL), cooled to 0° C. and filtered to get the corresponding enamide (7.4 g, 50.8%) as a yellow crystalline solid.
Step-3: A stirred mixture of the guanidine derivative (2 g, 9.6 mmol), the enamide derivative (1.88 g, 10.7 mmol) and NaOH (0.44 g, 11.0 mmol) in ethanol (27 mL) was refluxed at 90° C. for 48 h. The reaction mixture was then cooled and concentrated under reduced pressure to get a residue. The residue was taken in ethyl acetate (20 mL) and washed with water (5 mL). The organic and aqueous layers were separated and treated separately to get the corresponding ester and Intermediate C respectively. The aq. layer was cooled and acidified with 1.5 N HCl (pH˜3-4) when a white solid precipitated out. The precipitate was filtered, dried and excess water was removed by azeotropic distillation over toluene (2×10 mL) to get Intermediate C (0.5 g) as a pale yellow solid. ¹H NMR (DMSO-d₆, 400 MHz) δ (ppm): 2.32 (s, 3H), 7.36 (d, J=10.44 Hz, 1H), 7.53 (d, J=6.84 Hz, 1H), 760-7.72 (m, 2H), 8.26 (s, 1H), 8.57 (d, J=6.84 Hz, 1H), 8.64 (d, J=10.28 Hz, 1H), 8.70-8.78 (bs, 1H), 9.15 (s, 1H), 9.35 (s, 1H). The organic extract was washed with brine (3 mL), dried (Na₂SO₄) and concentrated under reduced pressure to get the ester of Intermediate C as crude solid. It was further purified by column chromatography (SiO₂, 60-120 mesh, MeOH/CHCl₃: 10/90) to get the ester of Intermediate C (0.54 g) as a yellow solid.

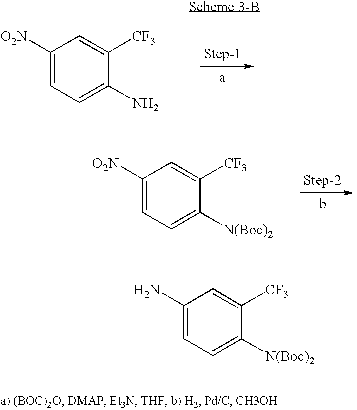

Step-1: To a stirring solution of the nitroaniline (0.15 g, 0.7 mmol) in THF (0.3 mL) was added Et₃N (0.11 mL, 0.73 mmol) and DMAP (0.05 g, 0.44 mmol). To it was added BOC anhydride (0.33 mL, 1.52 mmol) and the reaction was allowed to reflux for 5 h. The reaction mixture was then cooled, diluted with THF (15 mL) and washed with brine (5 mL). The organic phase was separated, dried over Na₂SO₄, filtered and concentrated under reduced pressure to get a crude mass. The crude product was further purified by column chromatography (SiO₂, 230-400 mesh, Hexane/EtOAc: 8/2) to get corresponding di-Boc protected aniline (0.25 g, 88%) as a white crystalline solid which was taken for next step without further purification.
Step-2: A solution of Boc protected aniline (0.25 g, 0.62 mmol) in MeOH (5 mL) was hydrogenated (H₂, 3 Kg) over 10% Pd/C (0.14 g, 0.13 mmol) at 20° C. for 12 h. The reaction mixture was passed through a short pad of Celite®, concentrated under reduced pressure to get the corresponding aniline as an off-white solid (0.18 g, 77.6%). 1H NMR (CD₃OD, 400 MHz) δ (ppm): 1.36 (s, 18H), 6.84-6.87 (m, 1H), 6.95-6.97 (m, 2H).

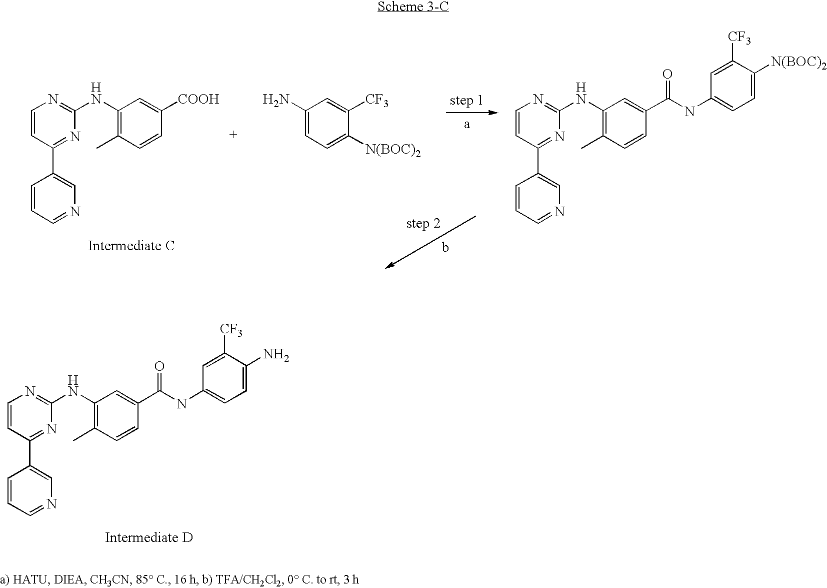

Step 1: Coupling of Intermediate C with diboc protected aniline in the presence of HATU, DIEA in acetonitrile can provide the corresponding amide
Step 2: Deprotection of the Boc groups to give Intermediate D can be accomplished by treating the amide with TFA in methylene chloride at 0° C. and then warming up to room temperature.

To a stirred solution of Intermediate D (0.1 g, 0.22 mmol) in THF (10 mL) at 0° C. was added Et₃N (0.033 g, 0.32 mmol) under N₂. Chloroacetyl chloride (0.029 g, 0.26 mmol) was added dropwise with stirring and the reaction mixture was allowed to come to room temperature and was stirred for 12 h. The reaction mixture was concentrated under reduced pressure to give a residue, which was taken in EtOAc (10 mL). This solution was washed with water (2 mL) and the aqueous layer was again extracted with EtOAc (2×10 mL). The EtOAc fractions were combined and were washed with brine (2 mL). Following drying over Na₂SO₄ and filtration the EtOAc solution and was concentrated under reduced pressure to give crude residue, which was then purified by column chromatography (SiO₂, 60-120 mesh, CHCl₃/MeOH: 9/1) to give III-14 (50 mg, 43%) as a pale yellow solid. 1H NMR (DMSO-d₆) δppm: 2.34 (s, 3H), 4.30 (s, 2H), 7.42-7.48 (m, 4H), 7.73-7.75 (m, 1H), 8.05-8.10 (m, 1H), 8.24 (d, J=2.2 Hz, 1H), 8.29 (s, 1H), 8.43 (d, J=8.04 Hz, 1H), 8.54 (d, J=5.16 Hz, 1H), 8.67 (dd, J=1.6 & 4.76 Hz, 1H), 9.16 (s, 1H), 9.26 (d, J=2.2 Hz, 1H), 9.89 (s, 1H), 10.50 (s, 1H); LCMS: m/e 541.2 (M+1)

### Target Inhibition

The capacity of Compound 3 to inhibit cKIT or PDGFR was assessed using the cKIT inhibition assay or PDGFR inhibition assay described in Example 1. The data showed that Compound 3 was a potent inhibitor of cKIT (IC₅₀=0.7 nM) and PDGFR (IC₅₀=9 nM). (Table 8)

TABLE 8
Compound 3 Inhibition Data

| Target | IC₅₀ (nM) |
|---|---|
| cKIT | 0.7 |
| PDGFR | 9 |

### PDGFR Mass Spectral Analysis

Mass spectral analysis of PDGFR-α in the presence of Compound 3 was performed. PDGFR-α (43 pmols) was incubated with Compound 3 (434 pmols) for 3 hours at 10× access prior to tryptic digestion. Iodoacetamide was used as the alkylating agent after compound incubation. For tryptic digests a 5 μl aliquot (7 pmols) was diluted with 10 μl of 0.1% TFA prior to micro C18 Zip Tipping directly onto the MALDI target using alpha cyano-4-hydroxy cinnamic acid as the matrix (5 mg/ml in 0.1% TFA:Acetonitrile 50:50).

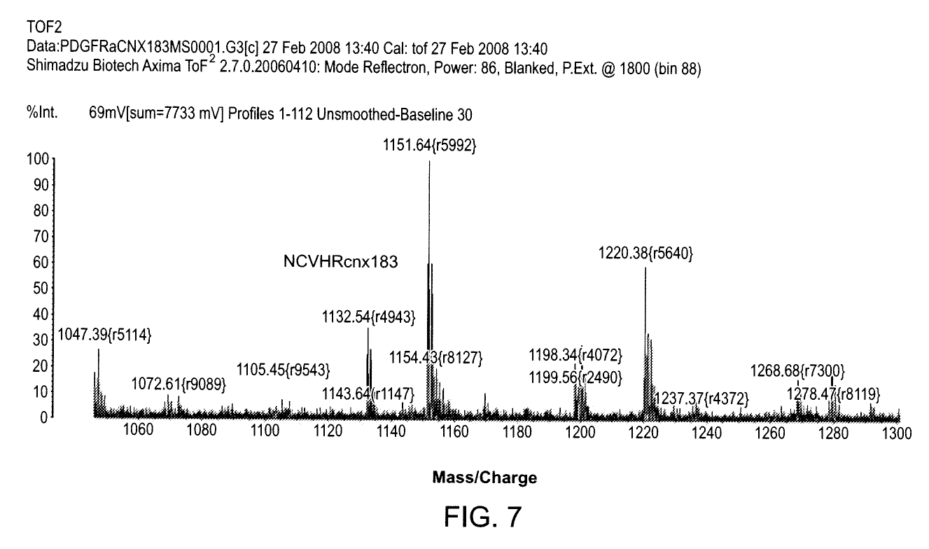

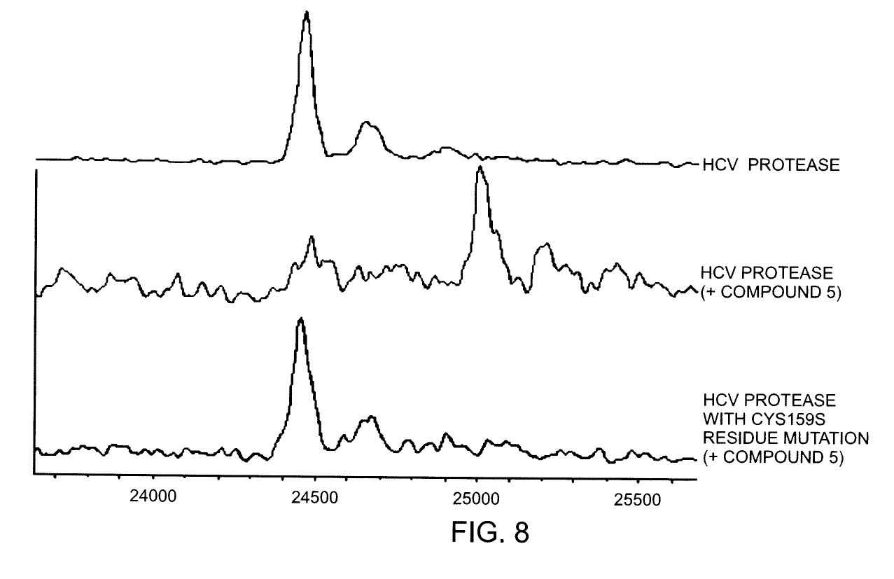

The tryptic digest was analyzed by mass spectrometer (MALDI-TOF). Mass spectral analysis of the tryptic digests was consistent with Compound 3 being covalently bound to PDGFR-α protein at Cys814. (FIG. 7) MS/MS analysis of the tryptic digests confirmed presence of the Compound 3 at Cys814.

c-KIT Mass Spectral Analysis

Mass spectral analysis of c-KIT in the presence of Compound 3 was performed. Specifically, c-KIT kinase (86 pmols) was incubated with Compound 3 (863 pmols) for 3 hours at 10× access prior to tryptic digestion. Iodoacetamide was used as the alkylating agent after compound incubation. For tryptic digests a 5 μl aliquot (14 pmols) was diluted with 10 ul of 0.1% TFA prior to micro C18 Zip Tipping directly onto the MALDI target using alpha cyano-4-hydroxy cinnamic acid as the matrix (5 mg/ml in 0.1% TFA:acetonitrile 50:50).

The tryptic digest was analyzed by mass spectrometer (MALDI-TOF). Mass spectral analysis of the tryptic digests was consistent with Compound 3 being covalently bound to c-KIT protein at two target cysteine residues Cys788 (major) and: Cys808 (minor).

cKIT Washout Assay

GIST430 cells (See, Bauer et al., Cancer Research, 66(18):9153-9161 (2006)) were seeded in a 6 well plate at a density of 8×10⁵ cells/well and treated with 1 μM compound 3 diluted in complete media for 90 minutes the next day. After 90 minutes, the media was removed and cells were washed with compound-free media. Cells were washed every 2 hours and resuspended in fresh compound-free media. Cells were collected at specified time-points, lysed in Cell Extraction Buffer (Invitrogen FNN0011) supplemented with Roche complete protease inhibitor tablets (Roche 11697498001) and phosphatase inhibitors (Roche 04 906 837 001) and 10 μg total protein lysate was loaded in each lane. c-KIT phosphorylation was assayed by western blot with pTyr (4G10) antibody and total kit antibody from Cell Signaling Technology. The results are depicted in Table 9 where it is shown that Compound 3 maintains c-KIT enzyme inhibition in GIST430 cells after “washout” at 0 hours and 6 hours.

TABLE 9
cKIT Wash Out Assay

| Treatment | Relative cKIT phosphorylation (%) |
|---|---|
| DMSO control | 100 |
| Coumpound 3 | 11 |
| 0 hours washout | |
| Compound 3 | 15 |
| 6 hours washout | |

### Example 3

### Irreversible VX-680

VX-680 is a potent reversible inhibitor of FLT3 kinase. Using the structure-based design algorithm described herein, VX-680 was rapidly and efficiently converted into an irreversible inhibitor of FLT-3.

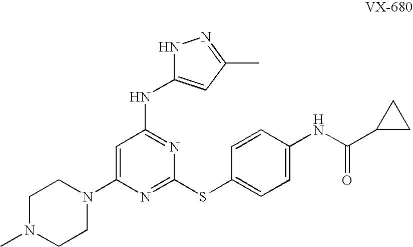

The binding mode of VX-680 to Flt3 was determined by inference from the binding mode of VX-680 with the related Aurora Kinase, as the crystal structure of the Aurora Kinase complex with VX-680 has been determined. A homology model of FLT3 was built using the x-ray structure of Aurora Kinase (pdbcode 2F4J) using the protein modeling component in Accelrys Discovery Studio (Discovery Studio v2.0.1.7347, Accelrys Inc). The alignment used for the model building was based upon the structural alignment of the x-ray complexes of FLT3 and Aurora kinase. The high structural similarity between these two proteins, and the high similarity of the binding site positions further supported the homology modeling strategy.

Structural alignment between FLT3 (1RJB) and Aurora Kinase/VX-680 complex (2FB4) with 256 structurally equivalent positions with an RMSD of 3k

| | | |
|---|---|---|
| Chain 1: | 230 | PNYDKWEMERTDITMKHKLGGGQYGEVYEGVWKK-----YSLTVAVKTLKEDTMEVEEFLKEAAVMKEI- |
| Chain 2: | 598 | EYDLKWEFPRENLEFGKVLGSGAFGKVMNATAYGISKTGVSIQVAVKMLKE----REALMSELKMMTQLG |
| Chain 1: | 294 | KHPNLVQLLGVCTREPPFYIITEFMTYGNLLDYLRECNRQEVNAVVLLYMATQISSAMEYLEKKNFIHRD |
| Chain 2: | 670 | SHENIVNLLGACTLSGPIYLIFEYCCYGDLLNYLRSKREKFLTFEDLLCFAYQVAKGMEFLEFKSCVHRD |
| Chain 1: | 364 | LAARNCLVGENHLVKVADFGLSRLMTGDTY-TAPAGAKFPIKWTAPESLAYNKFSIKSDVWAFGVLLWEI |
| Chain 2: | 812 | LAARNVLVTHGKVVKICDFGLARDIMSDSNYVVRGNARLPVKWMAPESLFEGIYTIKSDVWSYGILLWEI |
| Chain 1: | 433 | ATYGMSPYPGIDLS-QVYELLEKDYRMERPEGCPEKVYELMRACWQWNPSDRPSFAEIHQAFETMF |
| Chain 2: | 882 | FSLGVNPYPGIPVDANFYKLIQNGFKMDQPFYATEEIYIIMQSCWAFDSRKRPSFPNLTSFLGCQL |

Chain 1: human Aurora Kinase (SEQ ID NO: 5)
Chain 2: human RAF (SEQ ID NO: 6)

The homology model of Flt3 with VX680 identified six Cys residues in Flt3 that are within 20 angstroms of bound VX680 (Cys694, Cys695, Cys681, Cys828, Cys807, and Cys790). Then, 7 substitutable positions on the VX-680 template (Formula III-1) were explored in three-dimensions to determine which could be substituted with a warhead to covalent bond with one of the identified Cys residues in the FLT3 binding site. The warheads were built in three dimensions onto the VX-680 template using Discovery Studio, and the structures of the resulting compounds were checked to determine if the warheads could reach a Cys in the binding site.

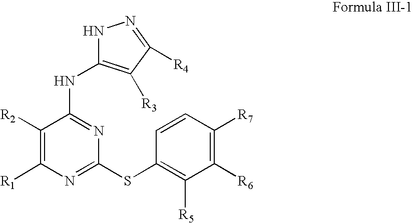

In order to sample the flexibility of the warheads and the side chain positions a standard molecular dynamics simulation of the warheads and side chain positions was performed, and checked to see if the warhead was within 6 angstroms of any of the identified Cys residues in the binding site. Standard settings were used in the Standard Dynamics Cascade Simulations protocol of Accelrys Discovery Studio v2.0.1.7347 (Accelrys Inc) protocol for molecular dynamics simulations. The coordinates of the non-warhead positions and the Cys main-chain atoms were held fixed during the molecular dynamics simulation.

This identified 3 template positions (R₄, R₆, and R₇) which were near Cys828. (Table 10) These template positions were subject to a final filter that required that the acrylamide reaction product could be formed between the candidate inhibitor and Cys828, which involved forming a bond of less than 2 angstroms using a standard molecular dynamics simulation. This constraint was successfully satisfied for all three positions. Compound 4, which contained an acrylamide at the R₇ position was synthesized.

TABLE 10

| Position | Placement | Distance from Cys | Can Bond be Formed |
|---|---|---|---|
| R₁ | | Too Far | |
| R₂ | Steric Clash | Clash | |
| R₃ | Steric Clash | | |
| R₄ | | OK | Yes |
| R₅ | | OK | No |
| R₆ | | OK | Yes |
| R₇ | | OK | Yes |

### Synthesis of Compound 4

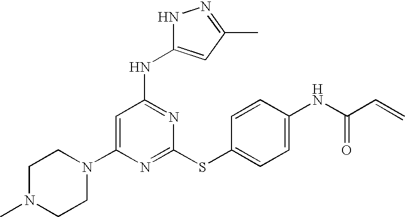

### Step 1,4,6-dichloro-2-methylsulfonyl pyrimidine

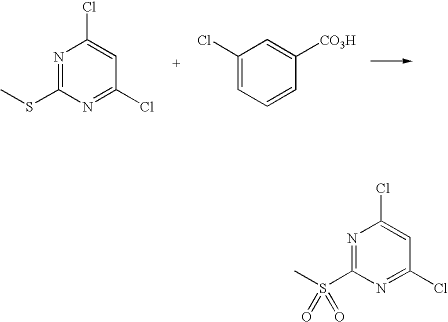

4,6-Dichloro-2-(methylthio)pyridine (24 g, 0.123 mol) was dissolved in 500 ml of CH₂Cl₂, under stirring and ice bath. Meta-chloroperoxybenzoic acid (about 0.29 mol) was added slowly in a period of 40 min. The reaction mixture was stirred for 4 h, was diluted with CH₂Cl₂, and was then treated with 50% Na₂S₂O₃/NaHCO₃ solution. The organic phase was washed with saturated aqueous NaCl, was dried over MgSO₄, and was then filtered. Removal of solvent under vacuum yielded about 24 g of the title compound as a light purple colored solid.

### Step 2. Tert-Butyl N-(4-mercaptophenyl)carbamate

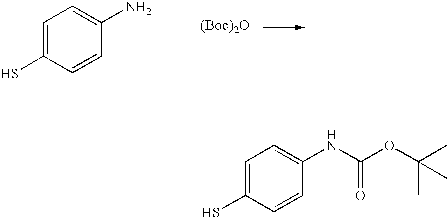

4-Aminothiophenol (25 g, 0.2 mol) was dissolved in 250 ml of EtOAc. The solution was cooled with an ice bath and di-t-butyldicarbonate (48 g, 0.22 mol) was added dropwise with stirring. After stirring for 1 h, saturated NaHCO₃ in water (200 ml) was added. The reaction mixture was stirred for overnight. The organic phase was washed with water, saturated aqueous NaCl solution, was dried over MgSO₄, and was then filtered. Removal of organic solvent under vacuum yielded about 68 g of yellow oil, which was treated with hexane to yield about 50 g of the title compound as a yellow solid.

### Step 3. Tert-butyl 4-(4,6-dichloropyrimidin-2-ylthio)phenylcarbamate

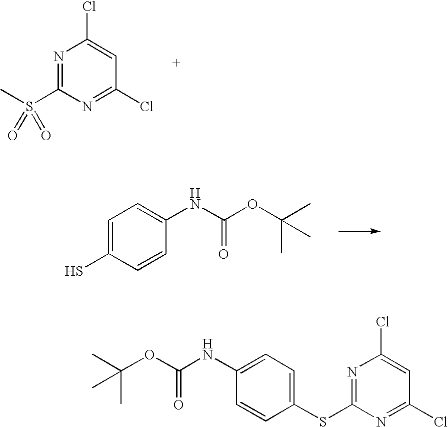

A mixture of tert-butyl N-(4-mercaptophenyl)carbamate (5 g, 0.022 mol) and 4,6-dichloro-2-methylsulfonylpyrimidine (5 g, 0.026 mol) in 150 ml of t-BuOH was heated at reflux for 1 h and then NaOAc (0.5 g) was added. The reaction was heated at reflux for an additional 14 h. Solvent was removed under vacuum and the residue was dissolved in ethyl acetate. The organic phase was washed successively with K₂CO₃ solution and saturated aqueous NaCl, was dried over MgSO₄, and was then filtered. Removal of the solvent yielded about 5 g of the title compound as yellow solid.

### Step 4. Tert-butyl 4-(4-chloro-6-(3-methyl-1H-pyrazol-5-ylamino)pyrimidin-2-ylthio)phenylcarbamate

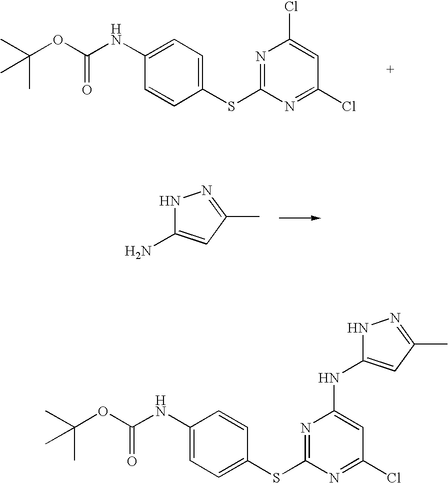

A solution of tert-butyl 4-(4,6-dichloropyrimidin-2-ylthio)phenylcarbamate (100 mg, 0.27 mmol), 3-methyl-5-amino-1H-pyrazol (28.7 mg, 0.3 mmol), diisopropylethylamine (41.87 mg), and NaI (48.6 mg, 0.324 mmol) in 1 ml of DMF was heated at 85° C. for 4 h. Following cooling and dilution with 20 mL of ethyl acetate, the organic phase was washed successively with water and saturated aqueous NaCl, was dried over MgSO₄, and was then filtered. Removal of solvent in vacuum yielded about 120 mg of crude product, which was purified by silica gel (30% EtOAc/hexanes) to yield 64 mg of the title compound.

### Step 5. Tert-butyl 4-(4-(3-methyl-1H-pyrazol-5-ylamino)-6-(4-methylpiperazin-1-yl)pyrimidin-2-ylthio)phenylcarbamate

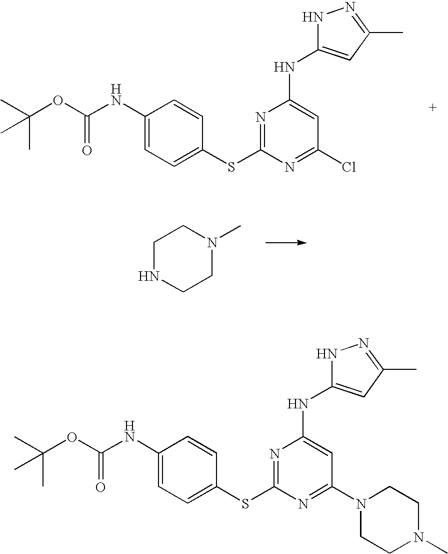

A mixture of tent-butyl 4-(4-chloro-6-(3-methyl-1H-pyrazol-5-ylamino)pyrimidin-2-ylthio)phenylcarbamate (61 mg, 0.14 mmol) and 1 ml of methylpiperazine was heated at 110° C. for 2 h. The reaction mixture was diluted with 20 mL ethyl acetate. The organic phase was washed with water, was dried over MgSO4, and was then filtered. Removal of solvent in vacuum yielded about 68.2 mg of crude product as light brown solid, which was purified by silica gel (30% EtOAc/hexanes) to give 49.5 mg of the title compound. MS (M+H⁺): 497.36.

### Step 6. 2-(4-Aminophenylthio)-N-(3-methyl-1H-pyrazol-5-ylamino)-6-(4-methylpiperazin-1-yl)pyrimidin-4-amine

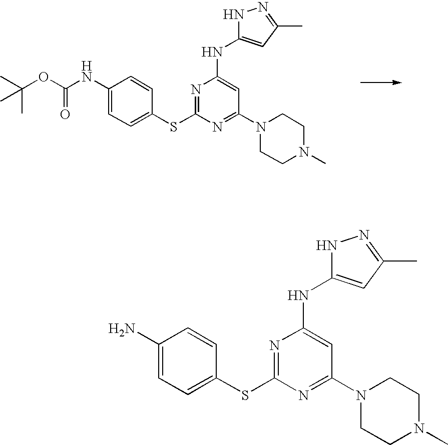

A solution of tert-butyl 4-(4-(3-methyl-1H-pyrazol-5-ylamino)-6-(4-methylpiperazin-1-yl)pyrimidin-2-ylthio)phenylcarbamate (44.5 mg) in 5 ml of MeOH was treated with 2 ml of 5N HCl. When TLC was showed that no starting material remained, the reaction mixture was diluted with ethyl acetate. The organic phase was washed with NaHCO₃, and saturated aqueous NaCl, was dried over MgSO₄, and was then filtered. Removal of solvent in vacuum gave about 32.1 mg of the title compound. ¹H NMR (300 MHz, DMSO-d₆): δ 11.68 (s, 1H), 9.65 (s, 1H), 9.25 (s, 1H), 7.60 (d, 2H), 7.45 (d, 2H), 6.00 (s, 1H), 5.43 (s, 1H), 2.38 (m, 4H), 2.20 (m, 2H), 2.05 (m, 2H), 1.52 (s, 6H), MS (M+H⁺): 397.18.

### Step 7. N-(4-(4-(3-Methyl-1H-pyrazol-5-ylamino)-6-(4-methylpiperiazin-1-yl)pyrimidin-2ylthio)phenyl)acrylamide

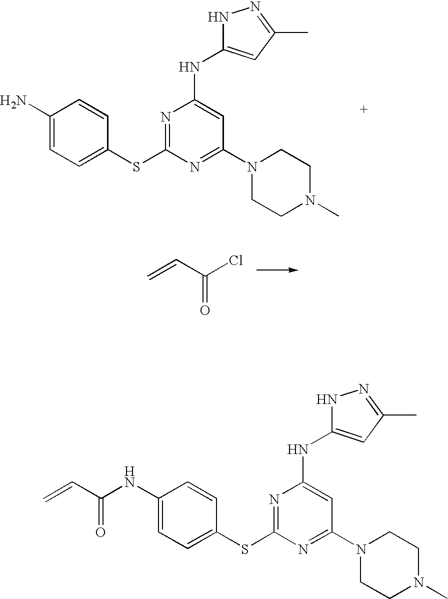

Acryloyl chloride (6.85 mL, 7.33 mg, 0.081 mmol) was added to a solution of 2-(4-aminophenylthio)-N-(3-methyl-1H-pyrazol-5-yl)-6-(4-methylpiperazin-1-yl)pyrimidin-4-amine (32.1 mg, 0.081 mmol) in 3 ml of CH₂Cl₂ at 0° C. After 30 min the reaction mixture was diluted with ethyl acetate. The organic phase was washed with NaHCO₃ solution, saturated aqueous NaCl solution, was dried over MgSO₄, and was then filtered. Removal of solvent yielded the crude product, which was purified by silica gel to give 20 mg of the product. MS (M+H⁺): 451.36.

### Biochemical Testing

Compound 4 had an IC50 of 2.2 nM for inhibition of FLT3 phosphorylation in the FLT3 biochemical assay. VX-680 had an IC50 of 10.7 nM in the assay.

### FLT3 Biochemical Assay

A continuous-read kinase assay was used to measure activity of compounds against active FLT-3 enzyme. The assay was performed in a mannor substantially similar to the method described by the vendor (Invitrogen, Carlsbad, Calif., world wide web invitrogen.com/content.cfm?pageid=11338). Briefly, 10× stocks of KDR from Invitrogen or BPS Bioscience (PV3660 or 40301) or FLT-3 (PV3182) enzymes, 1.13×ATP (AS001A) and Y9-Sox or Y12-Sox peptide substrates (KCZ1001) were prepared in 1× kinase reaction buffer consisting of 20 mM Tris, pH 7.5, 5 mM MgCl₂, 1 mM EGTA, 5 mM 3-glycerophosphate, 5% glycerol (10× stock, KBOO2A) and 0.2 mM DTT (DS001A). 5 μL of enzyme were pre-incubated in a Corning (#3574) 384-well, white, non-binding surface microtiter plate (Corning, N.Y.) for 30 min at 27° C. with a 0.5 pit volume of 50% DMSO and serially diluted compounds prepared in 50% DMSO. Kinase reactions were started with the addition of 45 pit of the ATP/Y9 or Y5-Sox peptide substrate mix and monitored every 30-90 seconds for 60 minutes at λₑₓ360/λₑₘ485 in a Synergy⁴ plate reader from BioTek (Winooski, Vt.). At the conclusion of each assay, progress curves from each well were examined for linear reaction kinetics and fit statistics (R², 95% confidence interval, absolute sum of squares). Initial velocity (0 minutes to 20+ minutes) from each reaction was determined from the slope of a plot of relative fluorescence units vs time (minutes) and then plotted against inhibitor concentration to estimate IC₅₀ from Log [Inhibitor] vs Response, Variable Slope model in GraphPad Prism from GraphPad Software (San Diego, Calif.).

[Reagent] used in optimized protocols:
[PDGFRα]=2-5 nM, [ATP]=60 μM and [Y9-Sox peptide]=10 μM (ATP Kₘₐₚₚ=61 μM)
[FLT-3]=15 nM, [ATP]=500 μM and [Y5-Sox peptide]=10 μM (ATP K_{Mapp}=470 μM)

### Mass Spectral Analysis

Flt3 was incubated with Compound 4 for 3 hrs at 100× excess prior to tryptic digestion. Iodoacetamide was used as the alkylating agent after compound incubation. For tryptic digests a 5 ul aliquot (7 pmols) was diluted with 10 ul of 0.1% TFA prior to micro C18 Zip Tipping directly onto the MALDI target using alpha cyano-4-hydroxy cinnamic acid as the matrix (5 mg/ml in 0.1% TFA:Acetonitrile 50:50).

The mass spec instrument was set in Reflectron mode with a pulsed extraction setting of 1800. Calibration was done using the Laser Biolabs Pep Mix standard (1046.54, 1296.69, 1672.92, 2093.09, 2465.20). For CID/PSD analysis the peptide was selected using cursors to set ion gate timing and fragmentation occurred at a laser power about 20% higher and He was used as the collision gas for CID. Calibration for fragments was done using the P14R fragmentation calibration for the Curved field Reflectron.

The modified form of the tryptic peptide with the sequence ICDFGLAR with Compound 4 attached formed a peak at 1344.73. The control digest did not show evidence of the 1344 peak that represents the Compound 4 modified peptide.

### Example 4

### Irreversible Boceprevir

Boceprevir is a potent reversible inhibitor of Hepatitis C Virus (HCV) protease. Using the structure-based design algorithm described herein, boceprevir was rapidly and efficiently converted from a reversible inhibitor into an irreversible inhibitor of HCV protease.

The coordinates for the x-ray complex of boceprevir bound to HCV protease (pdbcode 2008) were obtained from the protein data bank. The coordinates of boceprevir were extracted and all protein Cys residues within 20 angstroms of boceprevir were identified. This identified five residues Cys 16, Cys47, Cys52, Cys145 and Cys159. Then, 4 substitutable positions on the boceprevir template (Formula IV-1) were explored in three dimensions to determine which could be substituted with a warhead so that the warhead would form a covalent bond with the Cys in the boceprivir binding site. Acrylamide warheads were built in three dimensions onto the boceprevir template (Formula IV-1) using Accelrys Discovery Studio v2.0.1.7347 (Accelrys Inc, CA) and the structures of the resulting compounds were checked to see if the warheads could reach one of the identified Cys residues in the HCV protease binding site.

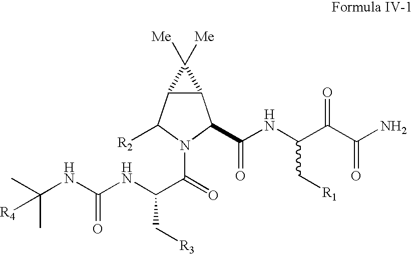

In order to sample the flexibility of the warheads and the side chain positions we performed a standard molecular dynamics simulation of the warheads and side chain positions and checked to see if the warhead was within 6 angstroms of any of the identified Cys residues in the binding site. This identified 2 template positions (R₁ and R₃) which were near Cys159. (Table 11) These two template positions were then subject to a final filter that required the reaction product to be formed between the candidate irreversible inhibitor and Cys 159, which involved forming a bond of less than 2 angstroms using a standard molecular dynamics simulation. This constraint left one template position, R₃. Compound 5 was synthesized and shown to have an IC₅₀_{_{—}}_{APP} of 1.3 μM in a biochemical assay (HCV Protease FRET Assay) and was shown to inhibit HCV replication in a replicon cellular assay with and EC50 of 230 nM.

TABLE 11

| Position | Placement | Distance to CYS | Can Bond be Formed |
|---|---|---|---|
| R₁ | | OK | No |
| R₂ | Steric Clash | Clash | |
| R₃ | | OK | Yes |
| R₄ | | Too Far | |

### Synthesis of Compound 5

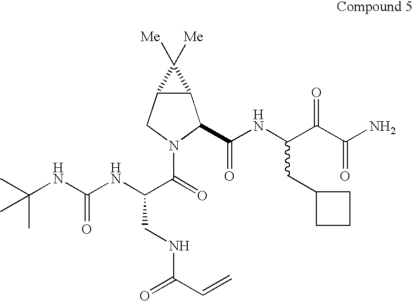

Compound 5 was prepared according to the steps and intermediates as described below.

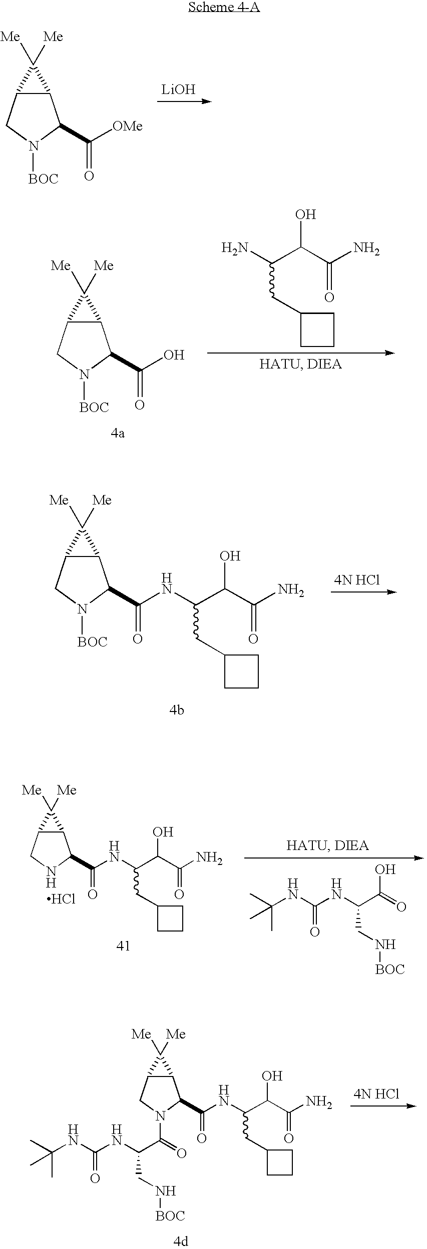

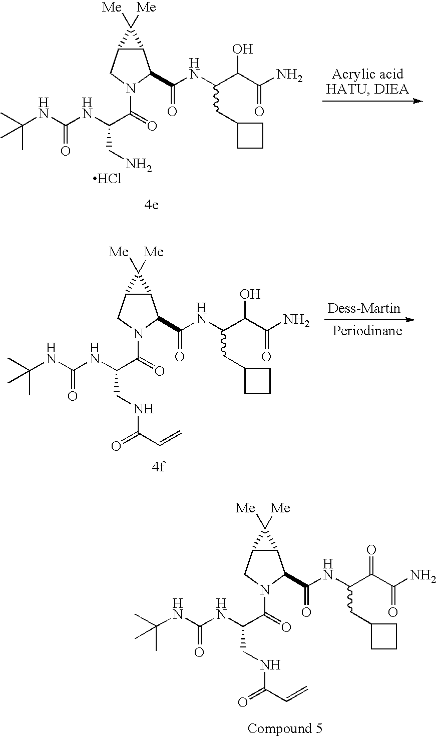

### Step 1: Intermediate 4a

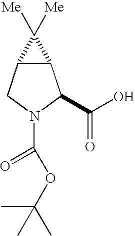

To a solution of (1R,2S,5S)-3-tert-butyl 2-methyl 6,6-dimethyl-3-azabicyclo[3.1.0]hexane-2,3-dicarboxylate (0.30 g, 1.1 mmol) in 4 mL THF/MeOH (1:1) was added 1 N aqueous LiOH solution (2.0 mL). After stirring at r.t. for 10 hours, the reaction mixture was neutralized with 1.0 N HCl. The organic solvents were evaporated under vacuum, and the remaining aqueous phase was acidified to pH-3 using 1.0 N HCl and was extracted with EtOAc. The organic layer was washed with brine, and was dried over anhydrous magnesium sulfate. After removal of solvent, 0.28 g of Intermediate 1a was obtained: MS m/z: 254.2 (ES−).

### Step 2: Intermediate 4b

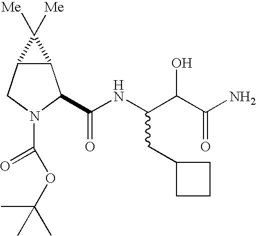

To a solution of the product of step 1 (0.28 g, 1.0 mmol) and 3-amino-4-cyclobutyl-2-hydroxybutanamide (0.27 g, 1.3 mmol) in 10.0 ml of anhydrous acetonitrile was added HATU (0.45 g, 1.2 mmol) and DIEA (0.5 ml, 3.0 mmol) at r.t. under stirring. TLC analysis indicated completion of the coupling reaction had occurred after 10 hours. A 50-ml portion of EtOAc was added in and the mixture was washed with aqueous NaHCO₃ and brine. The organic layer was separated and was dried over Na₂SO₄. After removal of solvent, the crude product was subject to chromatography on silica gel (eluents: EtOAc/hexane). A total of 0.4 g of the title compound was obtained (88%). MS m/z: 432.2 (ES+, M+Na).

### Step 3: Intermediate 4c

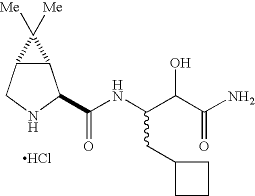

The product from step 2 (0.40 g, 1.0 mmol) was dissolved in 5 mL 4 N HCl in dioxane. The mixture was stirred at r.t. for 1 hour. After removal of solvents, a 10-mL portion of DCM was poured in followed by evaporation to dryness. This process of DCM addition followed by evaporation was repeated four times to give a residue solid which was used directly for the next step: MS m/z: 310.1 (M+H⁺).

### Step 4: Intermediate 4d

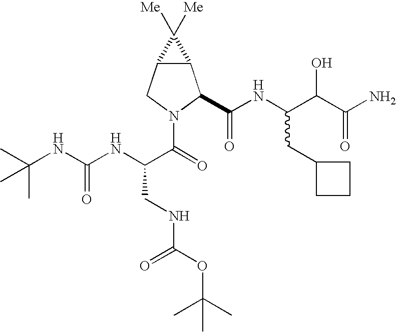

To a solution of the product from step 3 (0.10 g, 0.28 mmol) and (S)-3-(tert-butoxycarbonylamino)-2-(3-tert-butylureido)propanoic acid (0.10 g, 0.33 mmol) in 3.0 mL of anhydrous acetonitrile was added HATU (125 mg, 0.33 mmol) and DIEA (0.17 mL, 1.0 mmol) at r.t. under stirring. After one hour, 15 mL of EtOAc was added in and the mixture was washed with aqueous NaHCO₃ and brine. The organic layer was separated and was dried over Na₂SO₄. After removal of solvent, the crude product was subject to chromatography on silica gel (eluents: EtOAc/hexane) to afford 103 mg of the title compound (60%). MS m/z: 595.2 (M+H⁺).

### Step 5: Intermediate 4e

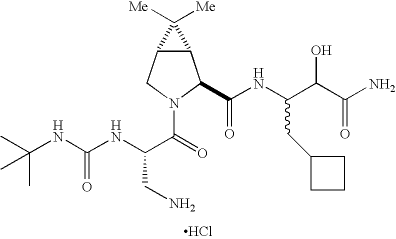

The product from step 4 (75 mg, 0.12 mmol) was dissolved in 3 mL of 4 N HCl in dixoxane and the reaction was stirred for 1 hour at RT. After removal of solvents, a 3-mL portion of DCM was poured in followed by evaporation to dryness. This process of DCM addition followed by evaporation was repeated three times to give a light brown solid and was used directly for the next step. MS m/z: 495.2 (M+H⁺).

### Step 6: Intermediate 4f

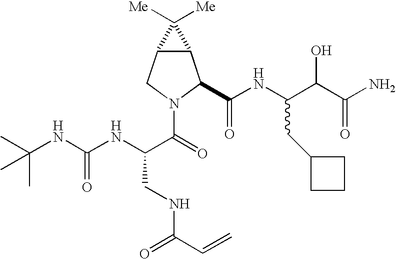

Acrylic acid (13.6 mg, 0.19 mmol) was coupled with the product from step 5 with HATU (65 mg, 0.17 mmol) following the procedure described in step 2 to afford the title compound (60 mg, crude). MS m/z: 549.3 (M+H⁺).

Step 7: The crude product from step 6 (60 mg, 0.11 mmol) was dissolved in 5 ml of dichloromethane followed by the addition of the Dess-Martin periodinane (60 mg, 0.15 mmol). The resulting solution was stirred for 1 h at room temperature. The solvent was then removed and the residue was subject to chromatography on silica gel (eluents: EtOAc/Heptanes) to provide 13 mg of Compound 5. MS m/z: 547.2 (M+H⁺).

### Mass Spectral Analysis

Mass spectrometric analysis of HCV in the presence of Compound 5 was performed using the following protocol: HCV NS3/4A wild type (wt) was incubated for 1 hr at a 10× fold access of Compound 5 to protein. 2 μl aliquots of the samples were diluted with 10 μl of 0.1% TFA prior to micro C4 ZipTipping directly onto the MALDI target using Sinapinic acid as the desorption matrix (10 mg/ml in 0.1% TFA:Acetonitrile 50:50). The instrument was set in linear mode using a pulsed extraction setting of 24,500 and apomyoglobin as the standard to calibrate the instrument. Compared to the protein without Compound 5, the protein incubated with Compound 5 reacted significantly to produce a new species which is approximately 547 Da heavier than HCV protease and consistent with the mass of Compound 5 at 547 Da.

Additional analysis of Compound 5 using a mutated form of HCV protease in which Cys159 was mutated to Ser, showed that Compound 5 did not modify the mutant HCV protease.

### Single Chain HCV Protease (wt) Peptide Expression and Purification

The single-chain proteolytic domain (NS4A₂₁₋₃₂-GSGS-NS₃₃₋₆₃₁) was cloned into pET-14b (Novagen, Madison, Wis.) and transformed into DH10B cells (Invitrogen). The resulting plasmid was transferred into Escherichia coli BL21 (Novagen) for protein expression and purification as described previously (1, 2). Briefly, the cultures were grown at 37° C. in LB medium containing 100 μg/mL of ampicillin until the optical density at 600 nm (OD600) reached 1.0 and were induced by addition of isopropyl-β-D-thiogalactopyranoside (IPTG) to 1 mM. After an additional incubation at 18° C. for 20 h, bacteria were harvested by centrifugation at 6,000×g for 10 min and resuspended in a lysis buffer containing 50 mM Na₃PO₄, pH 8.0, 300 mM NaCl, 5 mM 2-mercaptoethanol, 10% glycerol, 0.5% Igepal CA630, and a protease inhibitor cocktail consisting of 1 mM phenylmethylsulfonyl fluoride, 0.5 μg/mL leupeptin, pepstatin A, and 2 mM benzamidine. Cells were lysed by freezing and thawing, followed by sonication. Cell debris was removed by centrifugation at 12,000×g for 30 min. The supernatant was further clarified by passing through a 0.45-μm filter (Corning) and then loaded onto a HiTrap chelating column charged with NiSO₄ (Amersham Pharmacia Biotech). The bound protein was eluted with an imidazole solution in a 100-to-500 mM linear gradient. Selected fractions were run through Ni²⁺ column chromatography and were analyzed on a 10% sodium dodecyl sulfate (SDS)-polyacrylamide gel. The purified protein was resolved by electrophoresis in a 12% SDS-PAGE gel and then transferred onto a nitrocellulose membrane. The protein was analyzed by Western blot analysis using monoclonal antibodies against NS3. Proteins were visualized by using a chemiluminescence kit (Roche) with horseradish peroxidase-conjugated goat anti-mouse antibodies (Pierce) as secondary antibodies. The protein was aliquoted and stored at −80° C.

### Cloning and Expression of HCV Protease and C159S Variant

Mutant DNA fragments of NS4A/NS3 were generated by PCR and cloned into pET expression vector. After transformation into BL21 competent cells, the expression was induced with IPTG for 2 hours. The His-tagged fusion proteins were purified using affinity column followed by size exclusion chromatography.

### Biochemical Assay

An HCV Protease FRET Assay for HCV NS3/4A 1b Enzyme (IC50_APP) was used. The following protocol was used to generate “apparent” IC50 (IC50_APP) values. Without wishing to be bound by any particular theory, it is believed that IC50_APP, contrasted with IC50 values, may provide a more useful indication of time-dependent inhibition, and are thus more representative of binding affinity. The protocol is a modified FRET-based assay (v_{—}03) developed to evaluate compound potency, rank-order and resistance profiles against wild type and C159S, A156S, A156T, D168A, D168V, R155K mutants of the HCV NS3/4A 1b protease enzyme as follows: 10× stocks of NS3/4A protease enzyme from Bioenza (Mountain View, Calif.) and 1.13× 5-FAM/QXL™520 FRET peptide substrate from Anaspec (San Jose, Calif.) were prepared in 50 mM Tris-HCl, pH 7.5, 5 mM DTT, 2% CHAPS and 20% glycerol. 5 μl of each enzyme were added to Corning (#3575) 384-well, black, microtiter plates (Corning, N.Y.) after spotting a 0.5 μL volume of 50% DMSO and serially diluted compounds prepared in 50% DMSO. Protease reactions were immediately started after enzyme addition with the addition of 45 μL of the FRET substrate and monitored for 60-90 minutes at λex485/λem520 in a Synergy4 plate reader from BioTek (Winooski, Vt.). At the conclusion of each assay, progress curves from each well were examined for linear reaction kinetics and fit statistics (R², 95% confidence intervals, absolute sum of squares). Initial velocity (0 minutes to 15+ minutes) from each reaction was determined from the slope of a plot of relative fluorescence units vs. time (minutes) and then plotted against inhibitor concentration as a percent of the no inhibitor and no enzyme controls to estimate apparent IC50 from log [Inhibitor] vs Response, Variable Slope model in GraphPad Prism from GraphPad Software (San Diego, Calif.).

Compound 5 had inhibited HCV protease with an IC50 of 1.3 μM in this assay.

### Replicon Assay

The compounds were assayed to evaluate the antiviral activity and cytotoxicity of compounds using replicon-derived luciferase activity. This assay used the cell line ET (luc-ubi-neo/ET), which is a human Huh7 hepatoma cell line that contains an HCV RNA replicon with a stable luciferase (Luc) reporter and three cell culture-adaptive mutations.

The ET cell line was grown in Dulbecco's modified essential media (DMEM), 10% fetal bovine serum (FBS), 1% penicillin-streptomycin (pen-strep), 1% glutamine, 1% non essential amino acid, 400 μg/mL G418 in a 5% CO2 incubator at 37° C. All cell culture reagents were obtained from Invitrogen(Carlsbad). Cells were trypsinized (1% trypsin:EDTA) and plated out at 5×10³ cells/well in white 96-well assay plates (Costar) dedicated to cell number (cytotoxicity) or antiviral activity assessments. Compounds were added at six 3-fold concentrations each and the assay was run in DMEM, 5% FBS, 1% pen-strep, 1% glutamine, 1% non essential amino acid. Human interferon alpha-2b (PBL Biolabs, New Brunswick, N.J.) was included in each run as a positive control compound. Cells were processed 72 hr post compound addition when the cells are still subconfluent. Antiviral activity was measured by analyzing replicon-derived luciferase activity using the Steady-Glo Luciferase Assay System (Promega, Madison, Wis.) according to manufacturer's instruction. The number of cells in each well was determined by Cell Titer Blue Assay (Promega). Compound profile was derived by calculating applicable EC₅₀ (effective concentration inhibiting virus replication by 50%), EC₉₀ (effective concentration inhibiting virus replication by 90%), IC₅₀ (concentration decreasing cell viability by 50%) and SI₅₀ (selective index: EC₅₀/IC₅₀) values.

Compound 5 inhibited activity in this assay with an EC₅₀_{_{—}}_{APP} of 230 nM.

### Example 5

### Irreversible Inhibitor of Hepatitis C Virus Protease

Compound V-1 is a potent reversible inhibitor of HCV protease (IC₅₀_{_{—}}_{APP} of 0.4 nM in the biochemical assay described in Example 4.)

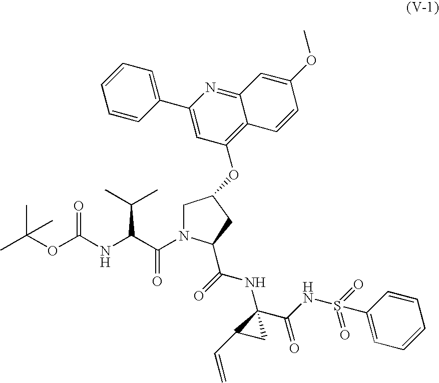

The coordinates for the x-ray complex of boceprevir bound to HCV protease (pdbcode 2008) were obtained from the protein databank (world wide web rcsb.org). The crystal structure of HCV protease has been determined with over 10 small molecules peptide-based inhibitors bound to it, and there are significant structural similarities in their binding modes. The structure of boceprevir was used to model-build the structure of V-1 in HCV protease using Discovery Studio.

All protein Cys residues within 20 angstroms of V-1 in the model were identified. This identified five residues Cys16, Cys47, Cys52, Cys145 and Cys159. Then, 4 substitutable positions on V-1 (using the V-2 template) were explored in three dimensions to determine which could be substituted with a warhead so that the warhead would form a covalent bond with an identified Cys residue in the HCV protease binding site. Warheads were built in three dimensions onto the template (Formula V-2) using a Discovery Studio (Accelrys Inc, CA) and the structures of the resulting compounds were checked to see if the warheads could reach one of the identified Cys residues in the binding site.

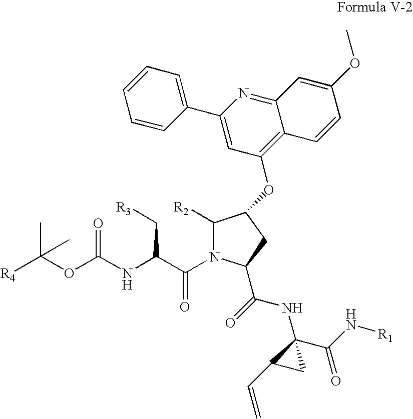

In order to sample the flexibility of the warheads and the side chain positions a standard molecular dynamics simulation of the warheads and side chain positions was performed and checked to see if the warhead was within 6 angstroms of any of the identified Cys residues in the binding site. This identified two template positions (R₁ and R₃) which were near Cys159. These two template positions were then subject to a final filter that required that the acrylamide reaction product could be formed with between the candidate inhibitor and Cys 159, which involved forming a bond of less than 2 angstroms using a standard molecular dynamics simulation. This constraint left one template position, R₃.

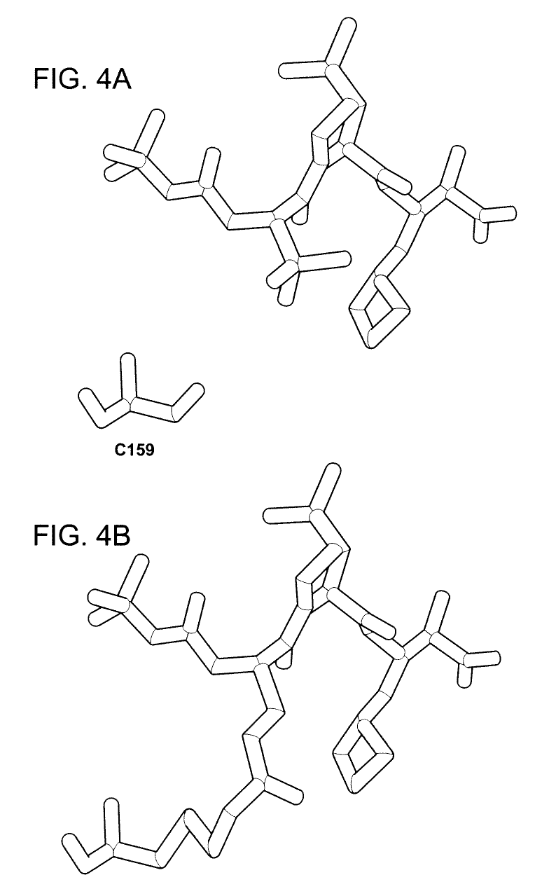

Compound 6 was synthesized and shown to be potent inhibitor of HCV protease (IC50 0.4 nM) and shown to modify HCV protease on Cys159 (FIG. 4).

### Synthesis of Compound 6

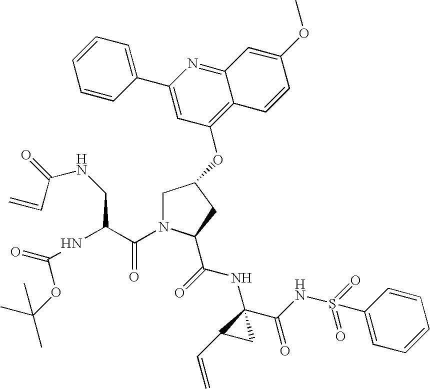

N-[(1,1-dimethylethoxy)carbonyl]-3-[(2-propenoyl)amino]-L-alanyl-(4R)-4-[(7-methoxy-2-phenyl-4-quinolinyl)oxy]-L-prolyl-1-amino-2-ethenyl-N-(phenylsulfonyl)-(1R,2S)-cyclopropanecarboxamide: The title compound was prepared according to the steps and intermediates as described below.

### Intermediate 5-1

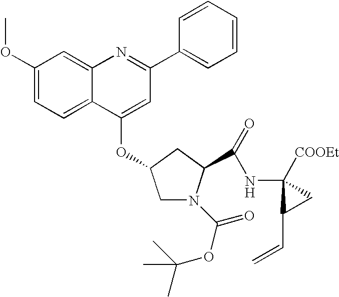

Ethyl-1-[[[(2S,4R)-1-[(1,1-dimethylethoxy)carbonyl]-4-[(7-methoxy-2-phenyl-4-quinolinyl)oxy]-2-pyrrolidinyl]carbonyl]amino]-2-ethenyl-(1R,2S)-cyclopropanecarboxylate: To a solution of (1R,2S)-1-amino-2-vinylcyclopropane carboxylic acid ethyl ester toluenesulfonic acid (2.29 g, 7.0 mmol) and N-Boc (2S,4R)-(2-phenyl-7-methoxy quinoline-4-oxo)proline (3.4 g, 7.3 mmol) in 100 ml of DCM was added HATU (3.44 g, 9.05 mmol) and then DIEA (3.81 ml, 21.9 mmol) under stirring. The mixture was stirred at r.t. for two hours. After the complete consumption of starting materials, the reaction mixture was washed with brine twice and dried over MgSO₄. After removal of solvent, the crude product was subject to chromatography on silica gel (hexane:EtOAc=2:1). 3.45 g of the title compound was obtained: R_{f} 0.3 (EtOAc:hexane=2:1); MS m/z: 602.36 (M+H⁺).

### Intermediate 5-2

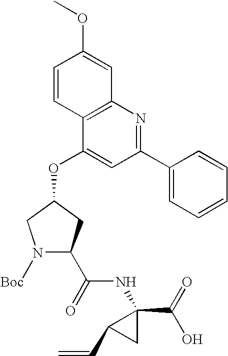

1-[[[(2S,4R)-1-[(1,1-dimethylethoxy)carbonyl]-4-[(7-methoxy-2-phenyl-4-quinolinyl)oxy]-2-pyrrolidinyl]carbonyl]amino]-2-ethenyl-(1R,2S)-cyclopropanecarboxylic acid: To a solution of the product of Intermediate 5-1 (1.70 g, 2.83 mmol) in 140 ml of THF/H₂O/MeOH (9:5:1.5) was added lithium hydroxide monohydrate (0.95 g, 22.6 mmol). After stirring at r.t. for 24 hours, the reaction mixture was neutralized with 1.0 N HCl. The organic solvents were evaporated under vacuum, and the remaining aqueous phase was acidified to pH˜3 using 1.0 N HCl and was extracted with EtOAc. The organic layer was washed with brine, and was dried over anhydrous magnesium sulfate. After removal of solvent, 1.6 g of the title compound was obtained: R_{f} 0.2 (EtOAc:MeOH=10:1); MS m/z: 574.36 (M+H⁺).

### Intermediate 5-3

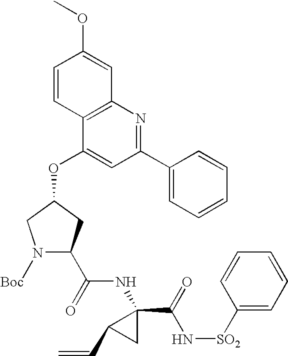

N-(1,1-dimethylethoxy)carbonyl)-(4R)-4-[(7-methoxy-2-phenyl-4-quinolinyl)oxy]-L-prolyl-1-amino-2-ethenyl-N-(phenylsulfonyl)-(1R,2S)-cyclopropanecarboxamide: To a solution of the product of Intermediate 5-2 (1.24 g, 2.16 mmol) in 20 ml of DMF was added HATU (0.98 g, 2.58 mmol) and DIEA (1.43 ml, 8.24 mmol), the mixture was stirred for one hour before adding a solution of benzenesulfonamide (1.30 g, 8.24 mmol), DMAP (1.0 g, 8.24 mmol) and DBU (1.29 g, 8.4 mmol) in 15 ml of DMF. Stirring continued for additional four hours. The reaction mixture was diluted with EtOAc and was washed with aqueous NaOAc buffer (pH˜5, 2×10 ml), NaHCO₃ solution and brine. After drying over MgSO₄ and removal of solvent a pure product precipitated by adding one portion of DCM. The filtrate was concentrated and the residue was subjected to chromatography on silica gel using hexane/EtOAc (1:1˜1:2). A total of 0.76 g of the title compound was obtained: R_{f} 0.3 (EtOAc:hexane=3:1), MS m/z: 713.45 (M+H⁺), 735.36 (M+Na⁺).

### Intermediate 5-4

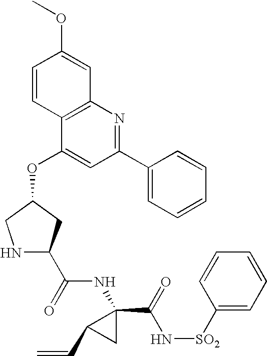

(4R)-4-[(7-methoxy-2-phenyl-4-quinolinyl)oxy]-L-prolyl-1-amino-2-ethenyl-N-(phenylsulfonyl)-(1R,2S)-cyclopropanecarboxamide: To a solution of the product from Intermediate 5-3 in 30 ml of DCM was added dropwise 15 ml of TFA. The mixture was stirred at r.t. for two hrs. After removal of solvents, a 20-ml portion of DCM was poured in followed by evaporation to dryness. This process of DCM addition followed by evaporation was repeated four times. Toluene (20 ml) was added and then removed by evaporation to dryness. Two repeats of this cycle gave a residue that solidified into 0.9 g white powder as TFA salt of the title compound. A small sample of the TFA salt was neutralized with NaHCO₃ to obtain the title compound: R_{f} 0.4 (DCM:MeOH=10:1); MS m/z: 613.65 (M+H⁺).

### Intermediate 5-5

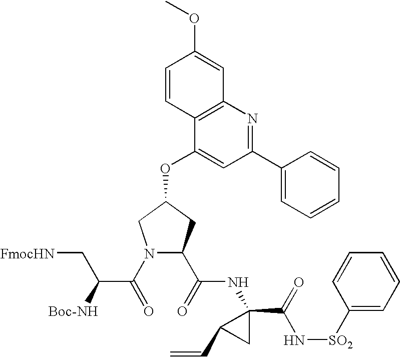

N-[(1,1-dimethylethoxy)carbonyl]-3-[[(9H-fluoren-9-ylmethoxy)carbonyl]amino]-L-alanyl-(4R)-4-[(7-methoxy-2-phenyl-4-quinoliny)oxy]-L-prolyl-1-amino-2-ethenyl-N-(phenylsulfonyl)-(1R,2S)-cyclopropanecarboxamide: To a solution of the product of Intermediate 5-4 (0.15 g, 0.178 mmol) and N-Boc-3-(Fmoc)amino-L-alanine (0.107 g, 0.25 mmol) in 3.0 ml of DMF was added HATU (85.1 mg, 0.224 mmol) and NMM (90.5 mg, 0.895 mmol) at r.t. under stirring. TLC analysis indicated completion of the coupling reaction had occurred after one hour. A 20-ml portion of EtOAc was poured in and the mixture was washed with a buffer (pH˜4, AcONa/AcOH), NaHCO₃ and brine, and was dried over MgSO₄. After removal of solvent, the crude oil product was subject to chromatography on silica gel (eluents: EtOAc/hexane). A total of 0.12 g of the title compound was obtained: R_{f} 0.4 (EtOAc:hexane=1:1); MS m/z: 1021.56 (M+H⁺).

### Intermediate 5-6

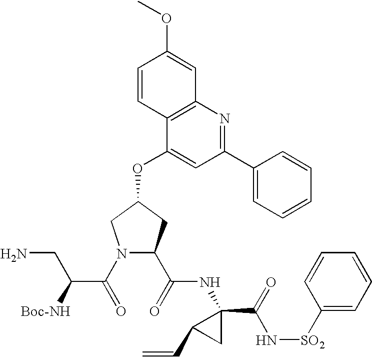

N-[(1,1-dimethylethoxy)carbonyl]-3-amino-L-alanyl-(4R)-4-[(7-methoxy-2-phenyl-4-quinolinyl)oxy]-L-prolyl-1-amino-2-ethenyl-N-(phenylsulfonyl)-(1R,2S)-cyclopropanecarboxamide: A solution of 110 mg of the product of Intermediate 5-5 (0.108 mmol) in 1 ml of DMF with 12% piperidine was stirred for 1.5 hours at r.t. and then was evaporated to dryness under high vacuum. The residue was trituated with hexane/ether (4:1) to yield 70 mg of the title compound: R_{f} 0.25 (EtOAc:MeOH=10:1); MS m/z: 798.9 (M+H⁺).

### Compound 6

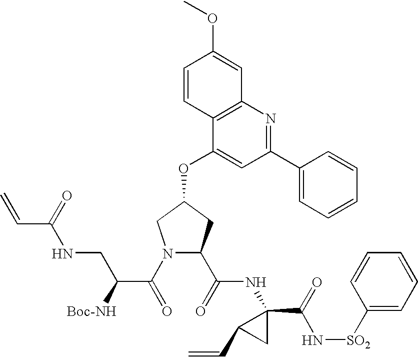

N-[(1,1-dimethylethoxy)carbonyl]-3-[(2-propenoyl)amino]-L-alanyl-(4R)-4-[(7-methoxy-2-phenyl-4-quinolinyl)oxy]-L-prolyl-1-amino-2-ethenyl-N-(phenylsulfonyl)-(1R,2S)-cyclopropanecarboxamide: Acryloyl chloride (11 uL, 0.132 mmol) was added dropwise at 0° C. to a stirred solution of 69 mg of the product from Intermediate 5-6 in 3 ml of DCM containing 3 eq. of triethylamine. The reaction mixture was stirred at r.t. for 1.5 hrs and then was diluted with 10 ml of DCM. The resulting solution was washed twice with brine and was dried over magnesium sulfate. Removal of solvent afforded the crude product, which was purified by chromatography on silica gel eluting first with hexane/EtOAc (1:3˜1:5) and then with DCM-methanol (50:1˜25:1)). A total of 36 mg of the title compound was obtained: R_{f} 0.25 (DCM:MeOH=25:1); MS m/z: 892.55 (M+H⁺).

### Mass Spectral Analysis

Mass spectral analysis showed modification of WT protease but not C159S mutant which supports specific modification of the targeted Cys by Compound 6.

Mass spectrometric analysis of HCV wild type or HCV variant C159S in the presence of test compound was performed. 100 pmols of HCV wild type (Bioenza CA) was incubated with compound for 1 hr and 3 hrs at 10-fold access of Compound 6 to protein. 1 μl aliquots of the samples (total volume of 4.24 ul) were diluted with 10 μl of 0.1% TFA prior to micro C4 ZipTipping directly onto the MALDI target using Sinapinic acid as the desorption matrix (10 mg/ml in 0.1% TFA:Acetonitrile 50:50). Analyses were performed on a Shimadzu Biotech Axima TOF² (Shimadzu Instruments) matrix-assisted-laser desorption/ionization Time-of-Flight (MALDI-TOF) mass spectrometer. The same procedure was carried out on 100 pmols of HCV C159S mutant of HCV protease for 3 hrs at 10-fold excess of Compound 6 to protein.

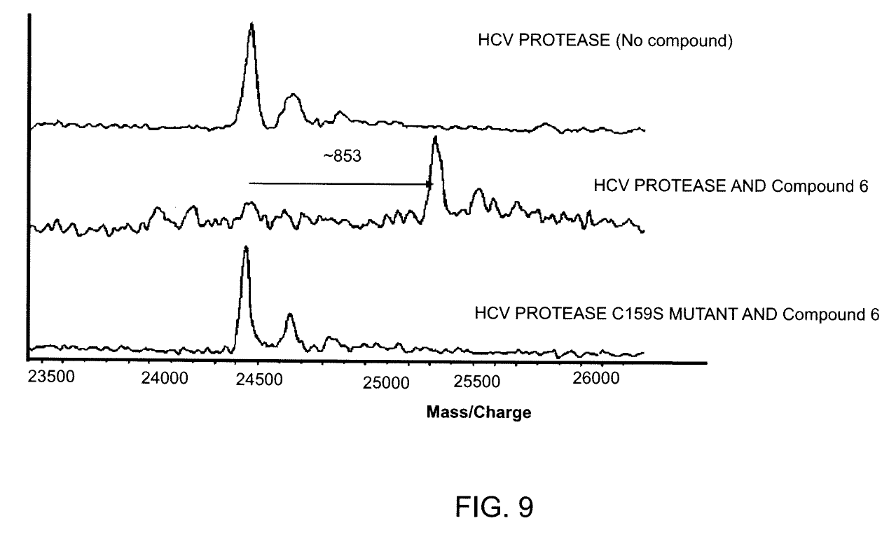

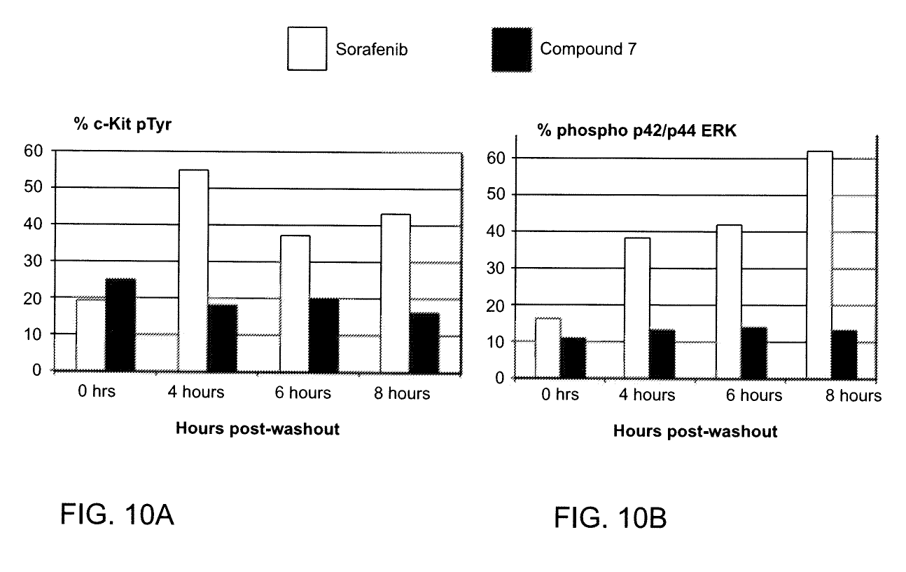

Intact HCV protein occurred at MH+ of 24465 with corresponding sinapinic (matrix) adducts occurring about 200 Da higher. A stoichiometric incorporation of Compound 6 (MW of 852 Da) occurred, producing a new mass peak which is approximately 850-860 Da higher (MH+ of 25320-25329). (FIG. 9) This is consistent with incorporation of a single molecule of Compound 6. Significant reaction occurred even after 1 hr at the 10× concentration of compound with nearly complete conversion after 3 hrs at the 10× concentration. The C159S variant form of the enzyme did not show any evidence of modification which confirms that the compound is modifying the Cys 159.

The mass spectral analysis confirmed that addition of Compound 6 to HCV protease resulted in a mass shift of 853 Daltons, demonstrating that an adduct of HCV protease with the compound was formed. Also, Compound 6 did not form an adduct with a mutated form of HCV protease in which Cys159 was changed to serine, as expected based upon the differential reactivities of Cys and Ser with the acrylamide warhead. These data demonstrate that the methods described herein have been used to design a specific irreversible inhibitor of HCV protease.

### Biochemical and Cellular Data

Compound 6 was tested in the biochemical and replicon assays described in Example 4. Compound 6 had an IC₅₀_{_{—}}_{APP} in the biochemical assay of 2.8 nM, and an EC50 in the replicon assay of 174 nM.

### Example 6

### Irreversible Sorafenib

Sorafenib is a potent reversible inhibitor of cKIT kinase domain. Using the design algorithm described herein, sorafenib was rapidly and efficiently convert into an irreversible inhibitor of cKIT.

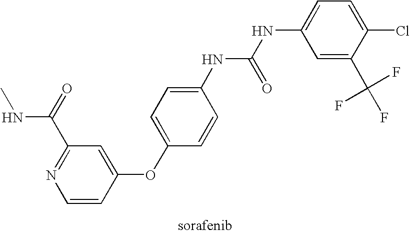

A homology model of cKIT kinase (Uniprot code: P10721) was produced using the x-ray structure of sorafenib bound to B-Raf as a template (pdbcode 1UWH). The homology model was built using the Build Homology module in Discovery Studio using the cKIT-B-RAF alignment shown below. Then, 10 substitutable positions on the sorafenib template (Formula VI-1) were explored in three dimensions to determine which could be substituted with a warhead so that the warhead would form a covalent bond with the Cys in the binding site. The methodology identified two template position (R₉ and R₁₀) and one Cys (Cys788) capable of forming a covalent bond using an acrylamide warhead. However the bond involving the R₉ position involved adoption of the cis-amide which is less preferred, while the bond involving the R₁₀ position was able to form the trans amide which is more preferred. Compound 7 was synthesized which tested the importance of having a warhead at the R₁₀ position.

| | |
|---|---|
| RAF | DDWEIPDGQITVGQRIGSGSFGTVYKGKWHGD--------VAVKMLNVTAPTPQQLQAFKNEVGVL |
| CKIT | HKWEFPRNRLSFGKTLGAGAFGKVVEATAYGLIKSDAAMTVAVKMLKPSAHL-TEREALMSELKVL |
| RAF | RKT-RHVNILLFMGYST-APQLAIVTQWCEGSSLYHHLHIIE-TK----------------FEMIK |
| CKIT | SYLGNHMNIVNLLGACTIGGPTLVITEYCCYGDLLNFLRRKRDSFICSKTSPAIMEDDELALDLED |
| RAF | LIDIARQTAQGMDYLHAKSIIHRDLKSNNIFLHEDLTVKIGDFGLATVLSG------------SIL |
| CKIT | LLSFSYQVAKGMAFLASKNCIHRDLAARNILLTHGRITKICDFGLARDIKNDSNYVVKGNARLPVK |
| RAF | WMAPEVIRMQDKNPYSFQSDVYAPGIVLYELMT-GQLPYSNINNRDQIIFMVGRGYLSPDLSKVRS |
| CKIT | WMAPESIFN---CVYTFESDVWSYGIFLWELFSLGSSPYPGMPVDSKFYKMIKEGFRMLSP----E |
| RAF | NCPKAMKRLMAECLKKKRDERPLFPQILASIELLARSLPK-- |
| CKIT | HAPAEMYDIMKTCWDADPLKRPTFKQIVQLIEKQISESTNHI |

RAF: human RAF (SEQ ID NO: 7)
CKIT: human CKIT (SEQ ID NO: 1)

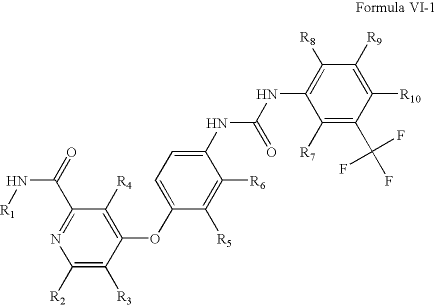

### Synthesis of Compound 7

### 4-(4-(3-(4-Acrylamido-3-(trifluoromethyl)phenyl)ureido)phenoxy)-N-methylpicolinamide

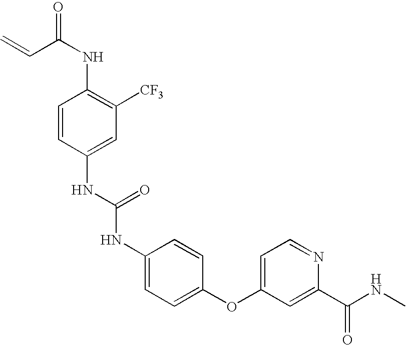

### Step 1. C,C′-Bis-tert-butyl N-4-amino-2-trifluoromethylphenyl)iminodicarbonate

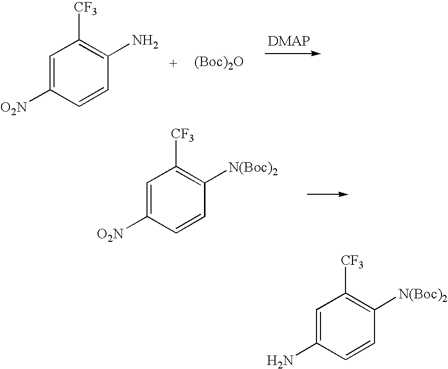

To a stirred solution of 4-nitro-2-trifluoromethylaniline (4.12 g, 20 mmol) in 1,4-dioxane (50 mL) was added 4-DMAP (1.22 g, 10 mmol) and Boc anhydride (13.13 g, 50 mmol) at room temperature. The reaction mixture was heated at 110° C. for 2 h. The reaction mixture was cooled, concentrated under reduced pressure and the residue was dissolved in EtOAc (25 mL). It was washed with 10% citric acid solution (5 mL), water (5 mL) and saturated aqueous NaCl (2 mL). Drying over Na₂SO₄ followed by concentration under reduced pressure offered a residue which was purified by column chromatography (SiO₂, 60-120, pet ether/ethyl acetate, 6/4) to give 5.3 g (13 mmol) of the bis-Boc intermediate as faint yellow solid. This material was dissolved in 50 mL methanol. To this solution under nitrogen atmosphere was added acetic acid (3 mL) followed by iron powder (1.71 g, 19.4 g-atom). The reaction mixture was heated at 70° C. for 2 h, was cooled to room temperature and was filtered through a Celite® bed. The filtrate was concentrated under reduced pressure and the residue was diluted with EtOAc (30 mL). It was washed with water (2 mL) and saturated aqueous NaCl (2 mL) and dried over Na₂SO₄. Concentration under reduced pressure gave a residue, which was further purified by column chromatography (SiO₂, 60-120, pet ether/ethyl acetate, 6/4) to give 3.19 g of the title compound as an off-white solid.

### Step 2. 4-(4-(3-(4-amino-3-(trifluoromethyl)phenyl)ureido)phenoxy)-N-methylpicolinamide

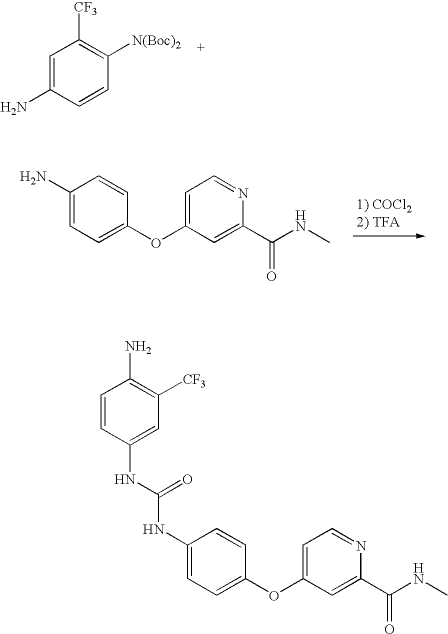

To a stirred solution of C,C′-bis-tert-butyl N-4-amino-2-trifluoromethylphenyl)iminodicarbonate (0.5 g, 1.32 mmol) and Et₃N (0.6 mL, 5.97 mmol) in toluene (5 mL) was added phosgene (20% solution in toluene, 0.91 mL, 1.85 mmol). The reaction mixture heated at reflux for 16 h and then was cooled to rt. 4-(4-Aminophenoxy)-N-methyl-2-pyridinecarboxamide, (0.32 g, 1.32 mmol) was added and the reaction mixture was heated at reflux for 2 h. After that the reaction mixture was quenched with water (5 mL) in a fume-hood, extracted with EtOAc (2×20 mL). The ethyl acetate extract was washed with saturated aqueous NaCl (15 mL), was dried over Na₂SO₄ and was concentrated under reduced pressure to 0.62 g of the title compound.

### Step 3. 4-(4-(3-(4-Acrylamido-3-(trifluoromethyl)phenyl)ureido)phenoxy)-N-methylpicolinamide

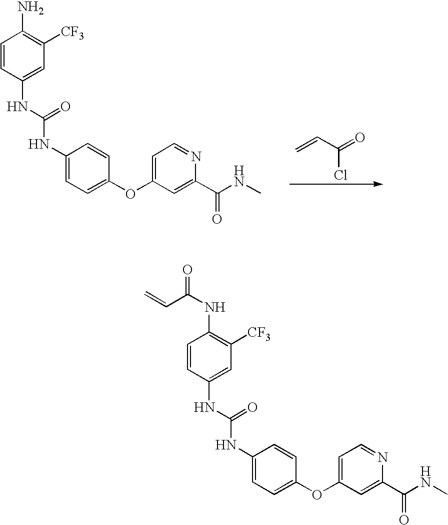

To a stirred solution of 4-(4-(3-(4-amino-3-(trifluoromethyl)phenyl)ureido)phenoxy)-N-methylpicolinamide (0.1 g, 0.22 mmol) and pyridine (0.035 g, 0.45 mmol) in DMF (5 mL) was added acryloyl chloride (0.03 g, 0.33 mmol) at 0° C. The reaction was allowed to come to rt and further stirred for 12 h, quenched with ice-cold water (10 mL) and extracted with EtOAc (2×20 mL). The ethyl acetate extract was washed with saturated aqueous NaCl solution (5 mL), dried over Na₂SO₄ and concentrated under reduced pressure to get crude CNX-43. The crude product was purified initially by neutral alumina column chromatography and then by prep. HPLC to give 18 mg of the title compound as a white solid. ¹H NMR (MeOD) δ ppm: 2.94 (s, 3H), 5.82 (d, J=10.0 Hz, 1H), 6.37 (dd, J=1.76 & 17.16 Hz, 1H), 6.50 (dd, J=10.28 & 17.16 Hz, 1H), 7.06 (dd, J=2.6 & 5.94 Hz, 1H), 7.11-7.15 (m, 2H), 7.45 (d, J=8.64 Hz, 1H), 7.56-7.61 (m, 3H), 7.67 (dd, J=2.24 & 8.48 Hz, 1H), 8.0 (s, 1H), 8.45-8.55 (m, 1H); LCMS: m/e 501 (M+2)

### Biochemical Testing

Sorafenib had an IC50 of 50.5 nM against inhibition of cKIT phosphorylation while Compound 7 had an IC50 of 31 nM against inhibition cKIT phosphorylation. Biochemical testing was performed using the assays described in Example 1 for cKIT.

### GIST882 Cellular Assay

GIST882 cells were seeded in a 6 well plate at a density of 8×10⁵ cells/well in complete media. The next day cells were treated with 1 uM compound diluted in complete media for 90 minutes. After 90 minutes, the media was removed and cells were washed with compound-free media. Cells were washed every 2 hours and resuspended in fresh compound-free media. Cells were collected at specified timepoints, lysed in Cell Extraction Buffer (Invitrogen FNN0011) supplemented with Roche complete protease inhibitor tablets (Roche 11697498001) and phosphatase inhibitors (Roche 04 906 837 001) and lysates were sheared by passing through a 28.5 gauge syringe 10 times each. Protein concentrations were measured and 10 μg total protein lysate was loaded in each lane. cKIT phosphorylation was assayed by western blot with pTyr (4G10) antibody and total kit antibody from Cell Signaling Technology.

Sorafenib and Compound 7 were tested for cellular activity in a GIST882 cell line at 1 micromolar. Both compounds inhibited cKIT autophosphorylation and also downstream signaling of ERK. In order to understand whether there was a prolonged inhibition with the irreversible inhibitor the cells were washed free of compound. For the reversible inhibitor, Sorafenib, the inhibitory activity of ckit and downstream signaling was overcome whereas the irreversible inhibition of Compound 7 persisted for at least 8 hours. This data supports the superiority in duration of action of the irreversible inhibitor Comopund 7 over the reversible inhibitor Sorafenib.

### Mass Spectral Analysis

c-KIT (15 pmols) was incubated with Compound 7 (150 pmols) for 3 hrs at 10× access prior to tryptic digestion. Iodoacetamide was used as the alkylating agent after compound incubation. A control sample was also prepared which did not have the addition of Compound 7. For tryptic digests a 2 μl aliquot (3.3 pmols) was diluted with 10 μl of 0.1% TFA prior to micro C18 Zip Tipping directly onto the MALDI target using alpha cyano-4-hydroxy cinnamic acid as the matrix (5 mg/ml in 0.1% TFA:Acetonitrile 50:50).

### Instrumental:

For tryptic digests the instrument was set in Reflectron mode with a pulsed extraction setting of 2200. Calibration was done using the Laser Biolabs Pep Mix standard (1046.54, 1296.69, 1672.92, 2093.09, 2465.20). For CID/PSD analysis the peptide was selected using cursors to set ion gate timing and fragmentation occurred at a laser power about 20% higher and He was used as the collision gas for CID. Calibration for fragments was done using the P14R fragmentation calibration for the Curved field Reflectron.

The peptide that was expected to be modified by Compound 7 has the sequence NCIHR, and was observed at MH+ of 1141.5. (The monoisotopic mass of Compound 7 was 499.15.) In comparison, the control digest of cKIT which did not include Compound 7 showed the complete absence of this mass peak. The data also suggested that there may have been modification of a peptide peptide that has the sequence ICDFGLAR.

### Example 7

### Irreversible Inhibitor of Hepatitis C Virus protease

As described in Example 5, Compound V-1 is a potent reversible inhibitor of HCV protease. Using a model-built structure of V-1 in HCV protease (see, Example 5), all protein Cys residues within 20 angstroms of V-1 in the model were identified. This identified five residues Cys16, Cys47, Cys52, Cys145 and Cys159. Then, 4 substitutable positions on V-1 that could be substituted with an enone warhead so that the warhead would form a covalent bond with an identified Cys residue in the HCV protease binding site were explored in three dimensions. The warheads were built in three dimensions onto the template (Formula V-2) using Discovery Studio (Accelrys Inc, CA) and the structures of the resulting compounds were checked to see if the warheads could reach one of the identified Cys residues in the binding site.

In order to sample the flexibility of the warheads and the side chain positions a standard molecular dynamics simulation of the warheads and side chain positions was performed and checked to see if the warhead was within 6 angstroms of any of the identified Cys residues in the binding site. This identified two template positions (R₁ and R₃) which were near Cys159. These two template positions were then subject to a final filter that required that the enone reaction product could be formed with between the candidate inhibitor and Cys159, which involved forming a bond of less than 2 angstroms using a standard molecular dynamics simulation. This constraint left one template position, R₃.

Compound 8 was synthesized and shown to be potent inhibitor of HCV protease (IC₅₀_{_{—}}_{APP}<0.5 nM) and shown to modify HCV protease on Cys159.

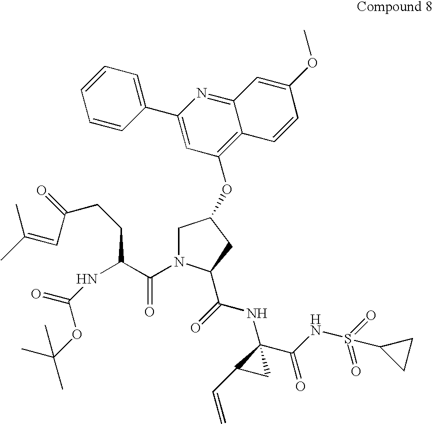

### Synthesis of Compound 8

### Compound 8

tert-butyl-(S)-1-((2S,4R)-2-((1R,2S)-1-(cyclopropylsulfonylcarbamoyl)-2-vinylcyclopropylcarbamoyl)-4-(7-methoxy-2-phenylquinolin-4-yloxy)pyrrolidin-1-yl)-7-methyl-1,5-dioxooct-6-en-2-ylcarbamate: The title compound was prepared according to the steps and intermediates as described below:

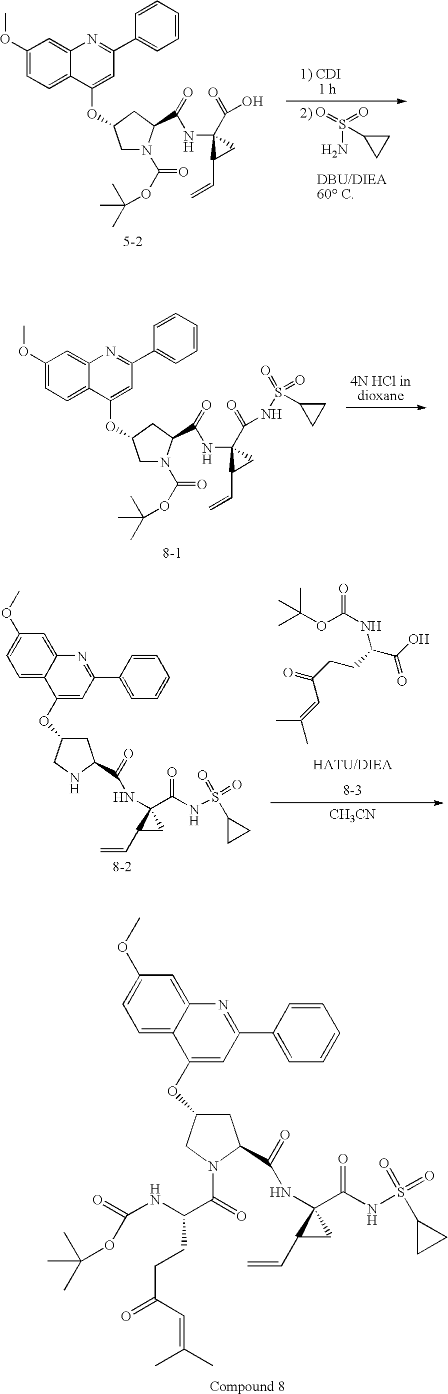

### Intermediate 8-1:

To a solution of Intermediate 5-2 (0.9 g, 1.57 mmol) in 6 ml of DMF was added CDI (0.28 g, 1.7 mmol). The mixture was stirred for one hour before adding a solution of cyclopropylsulfonamide (0.25 g, 2.0 mmol), DBU (0.26 ml, 1.8 mmol) and DIEA (0.9 ml, 5 mmol) in 2 ml of DMF. The resulting mixture was stirred at 60° C. for 10 hours. The reaction mixture was diluted with EtOAc and was washed with aqueous NaOAc buffer (pH˜5, 2×10 ml), NaHCO₃ solution and brine. After drying over Na2SO₄ and removal of solvent, the residue was subjected to chromatography on silica gel using hexane/EtOAc (1:1˜1:2). A total of 0.8 g of Intermediate 8-1 was obtained: R_{f} 0.3 (EtOAc:hexane=3:1), MS m/z: 677.2 (M+H⁺).

### Intermediate 8-2:

Intermediate 8-1 (0.8 g, 1.18 mmol) was dissolved in 5 ml of 4N HCl in dixoxane and the reaction was stirred for 1 hour at RT. After removal of solvents, a 20-ml portion of DCM was poured in followed by evaporation to dryness. This process of DCM addition followed by evaporation was repeated three times to give Intermediate 8-2 as its HCl salt. MS m/z: 577.2 (M+H⁺).

### Intermediate 8-3:

To a solution of N-Boc-pyroglutamic acid (0.23 g 1.0 mmol) in 10.0 ml of anhydrous THF was added 2-methylprop-1-enyl)magnesium bromide (0.5 M in THF, 5 mL, 2.5 mmol) at −78° C. slowly. The reaction mixture was stirred for 1 h at −78° C. 1 N HCl (2.5 ml) aqueous solution was added and the mixture was slowly warmed up to RT. The pH was adjusted to ˜3 by 1 N HCl. The THF was then removed under vacuum and the remaining aqueous was extracted by DCM (3×20 mL). The organic layer was dried over Na₂SO₄, filtered and the solvent was removed to provide the crude product.

### Compound 8

### tert-butyl-(S)-14(2S,4R)-2-((1R,2S)-1-(cyclopropylsulfonylcarbamoyl)-2-vinylcyclopropylcarbamoyl)-4-(7-methoxy-2-phenylquinolin-4-yloxy)pyrrolidin-1-yl)-7-methyl-1,5-dioxooct-6-en-2-ylcarbamate: The title compound was made by coupling Intermediate 8-2 and Intermediate 8-3 using HATU following the coupling reaction described for Intermediate 5-5 in the synthesis of Compound 6.

A total of 70 mg of the title compound was obtained (65%): R_{f} 0.5 (EtOAc);

MS m/z: 844.2 (M+H⁺).

### Biochemical Data

Compound 8 was tested in the biochemical assay described in Example 4, and shown to be potent inhibitor of HCV protease (IC₅₀_{_{—}}_{APP}<0.5 nM)

### Mass Spectral Analysis

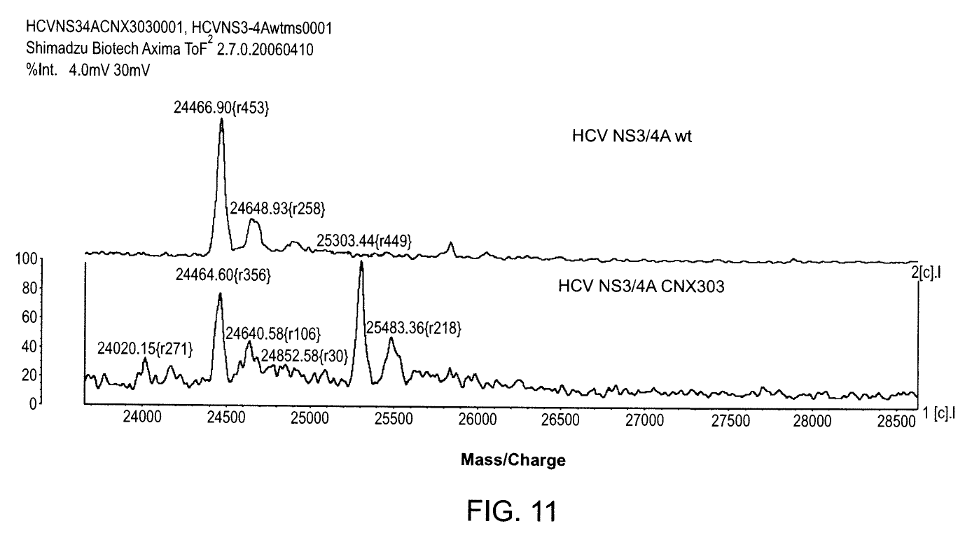

Mass spectrometric analysis was performed as described in Example 5. The analysis confirmed that addition of Compound 8 to HCV protease resulted in a mass shift of approximately 844 Daltons, demonstrating that an adduct of HCV protease with the compound was formed. (FIG. 11)

### Example 8

### Improved Potency Through Covalency

This example demonstrates application of the design algorithm and method to design potent irreversible inhibitors starting from reversible inhibitors with moderate or weak potency.

8.A. Inhibitor of Btk kinase

Compound 9 is a weak reversible inhibitor of Btk kinase (IC₅₀ 8.6 μM in the biochemical assay, and). Using the structure-based design algorithm described herein, Compound 9 was rapidly and efficiently converted into an irreversible inhibitor of Btk.

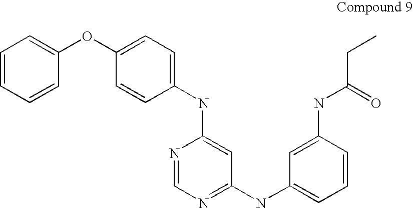

The binding mode of Compound 9 in Btk was obtained through the docking method using the Btk apo structure (pdb code: 1K2P) and the co-crystal structure of EGFR inhibitor (pdb code: 2RGP) with the protein modeling component in Discovery Studio (Discovery Studio v2.0.1.7347, Accelrys Inc).

The binding model of Compound 9 with Btk identified five Cys residues that were within 20 angstroms (Cys464, Cys481, Cys502, Cys506, and Cys527) of Compound 9. In the three dimensional structures, however, four (Cys464, Cys502, Cys506, and Cys527) out of the five cysteines were blocked by side chains or the protein backbone. Those cysteines are not easily accessible due to the steric clashes. Therefore, only one cysteine (Cys481) was reachable and within a preferred distance. One substitutable position on the Compound 9 template was explored in three dimensions (R₁ in Formula VIII-1). The warhead (acrylamide) was built onto the Compounds 9 template using Discovery Studio, and the structure of the resulting compound was docked into the Btk using Accelrys Discovery Studio v2.0.1.7347 (Accelrys Inc). The final three dimensional structure was checked to determine if the warhead could reach a Cys in the binding (was no more than 6 angstroms from a Cys).

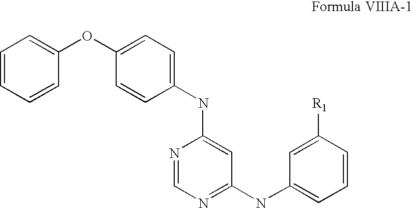

This approach confirmed that the selected R₁ position on the Compounds 9 template was near Cys481, and that the distance was less than 6 angstrom. The acrylamide reaction product was formed between the candidate inhibitor and Cys481, which involved forming a bond of less than 2 angstroms using a standard molecular dynamics simulation. This constraint was successfully satisfied for this position.

Using the methods and assays described below, Compound 10, which contains an acrylamide at the R₁ position, was synthesized and shown to be a potent inhibitor of Btk kinase with an IC₅₀ 1.8 nM in the biochemical assay. This is a significant improvement in potency relative to Compound 9 (IC₅₀ 8.6 μM). The activity of Compound 10 was also assessed in a Ramos cellular assay. Because Compound 9 was such a week inhibitor of Btk in the biochemical assay, it was not expected to have any inhibitory activity in the cellular assay. However, when used at a concentration of 1 μM, Compound 10 showed 85% inhibition of Btk signaling in Ramos cells. These data show that the algorithm and method of the invention were used to improve the potency of a weak reversible inhibitor (Compound 9) and that the potent irreversible inhibitor (Compound 10) had activity in cells.

### Synthesis of Compound 10

### N-{3-[6-(4-phenoxyphenylamino)-pyrimidin-4-ylamino]phenyl}-2 propenamide

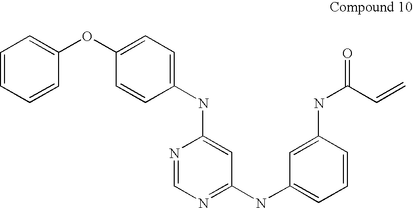

A solution of 3-[6-(4-phenoxyphenylamino)-pyrimidin-4-ylamino]phenylamine (250 mg, 0.7 mmol) and triethylamine (180 mg, 1.75 mmol) in 5 mL of THF was stirred at RT. Acryloyl chloride (80 mg, 0.9 mmol) was added into the reaction mixture and it was stirred at RT for 1 h. The solvent was removed by vacuum evaporation and the crude product was purified by flash chromatography on silica gel with EtOAc/DCM solvent system to afford 115 mg (40% yield) of the title compound as a light colored solid. MS (m/z): MH⁺=424. ¹H NMR (DMSO): 9.14 (s, 1H), 9.10 (s, 1H), 8.22 (s, 1H), 7.89 (s, 1H), 7.52 (d, 2H, J=9.0 Hz), 7.35-6.92 (m, 11H), 6.42 (dd, 1H, J₁=10.1 Hz, J₂=16.9 Hz), 622 (dd, 1H, J₁=1.9 Hz, J₂=16.9 Hz), 6.12 (s, 1H), 5.70 (dd, 1H, J₁=1.9 Hz, J₂=10.1 Hz) ppm.

### Omnia Assay Protocol for Potency Assessment Against Btk

The protocol below describes continuous-read kinase assays to measure inherent potency of compounds against active forms of Btk enzyme. The mechanics of the assay platform are best described by the vendor (Invitrogen, Carlsbad, Calif.) on the world wide web at invitrogen.com/site/us/en/home/Products-and-Services/Applications/Drug-Discovery/Target-and-Lead-Identification-and-Validation/KinaseBiology/KB-Misc/Biochemical-Assays/Omnia-Kinase-Assays.html. Briefly, 10× stocks of 5 nM Btk from Invitrogen, 1.13×40 μM ATP (AS001A) and 10 μM (ATP KMapp ˜36 mM) Tyr-Sox conjugated peptide substrates (KCZ 1001) were prepared in 1× kinase reaction buffer consisting of 20 mM Tris, pH 7.5, 5 mM MgCl₂, 1 mM EGTA, 5 mM β-glycerophosphate, 5% glycerol (10× stock, KBOO2A) and 0.2 mM DTT (DS001A). 5 μL of enzyme was pre-incubated in a Corning (#3574) 384-well, white, non-binding surface microtiter plate (Corning, N.Y.) for 30 min. at 27° C. with a 0.5 μL volume of 50% DMSO and serially diluted compounds prepared in 50% DMSO. Kinase reactions were started with the addition of 45 μL of the ATP/Tyr-Sox peptide substrate mix and monitored every 30-90 seconds for 60 minutes at λₑₓ360/λₑₘ485 in a Synergy⁴ plate reader from BioTek (Winooski, Vt.). At the conclusion of each assay, progress curves from each well were examined for linear reaction kinetics and fit statistics (R², 95% confidence interval, absolute sum of squares). Initial velocity (0 minutes to ˜30 minutes) from each reaction was determined from the slope of a plot of relative fluorescence units vs time (minutes) and then plotted against inhibitor concentration to estimate IC₅₀ from log [Inhibitor] vs Response, Variable Slope model in GraphPad Prism from GraphPad Software (San Diego, Calif.).

### Btk Ramos Cellular Assay

Compounds CNX-85 was assayed in Ramos human Burkitt lymphoma cells. Ramos cells were grown in suspension in T225 flasks, spun down, resuspended in 50 mls serum-free media and incubated for 1 hour. Compound was added to Ramos cells in serum free media to a final concentration of 1, 0.1, 0.01, or 0.001 μM. Ramos cells were incubated with compound for 1 hour, washed again and resuspended in 100 ul serum-free media. Cells were then stimulated with 1 μg of goat F(ab′)2 Anti-Human IgM and incubated on ice for 10 minutes to activate B cell receptor signaling pathways. After 10 minutes, the cells were washed once with PBS and then lysed on ice with Invitrogen Cell Extraction buffer. 16 μg total protein from lysates were loaded on gel and blots were probed for phosphorylation of the Btk substrate PLCγ2.

8.B. Inhibitor of HCV Protease

Compound 11 is a weak reversible inhibitor of HCV protease (IC₅₀ of 165 nM in the biochemical assay). Using the structure-based design algorithm described herein, Compound 11 was rapidly and efficiently converted into an irreversible inhibitor of HCV protease.

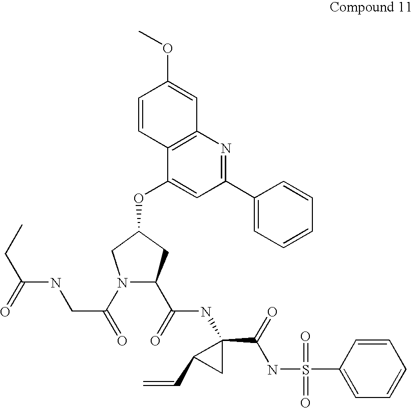

The crystal structures of HCV Protease in complex with over 10 small molecules peptide-based inhibitors have been determined, and there are significant structural similarities in the binding modes of the inhibitors. The x-ray structure of the complex with boceprevir (pdbcode 2OC8) was obtained from the protein databank (world wide web rcsb.org) and used to model-build the structure of Compound 11 in HCV protease using Discovery Studio.

All Cys residues of HCV protease within 20 angstroms in the docked compound in the model were identified. This identified five residues Cys 16, Cys47, Cys52, Cys145 and Cys159. Then, one substitutable position on the Compound 11 template (R₁ in Formula VIIIB-1) was explored in three dimensions to determine if it could be substituted with a warhead so that the warhead would form a covalent bond with an identified Cys residue in the HCV protease binding site. The warhead (acrylamide) was built in three dimensions onto the template (Formula VIIIB-1) and the rest of the reversible inhibitor was kept unchanged. The structure of the resulting compound was checked to see if the warhead could reach one of the identified Cys residues in the binding site.

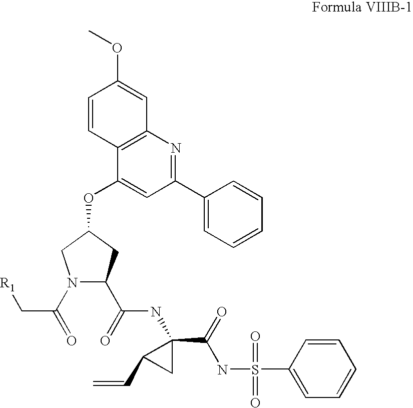

In order to sample the flexibility of the warhead and the side chain positions, a standard molecular dynamics simulation of the warhead and side chain positions was performed and checked to see if the warhead was within 6 angstroms of any of the identified Cys residues in the binding site. This approach confirmed that the R₁ position on the template was near Cys159. This position was then subject to a final filter that required that the acrylamide reaction product could be formed between the candidate inhibitor and Cys 159, which involved forming a bond of less than 2 angstroms using a standard molecular dynamics simulation. These constraints were met by Compound 12.

As described below, Compound 12 was synthesized and shown to be potent inhibitor of HCV protease.

### Synthesis of Compound 12

### (2S,4R)-1-(2-acrylamidoacetyl)-4-(7-methoxy-2-phenylquinolin-4-yloxy)-N-((1R,2S)-1-(phenylsulfonylcarbamoyl)-2-vinylcyclopropyl)pyrrolidine-2-carboxamide

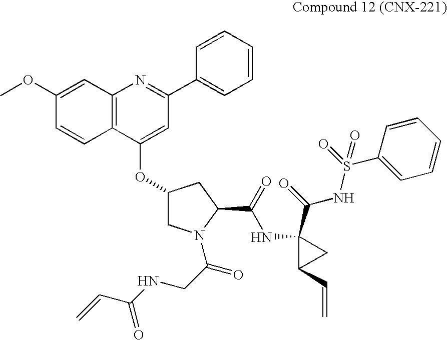

The title compound was prepared according to the steps and intermediates as described below.

### Intermediate 8.B-1

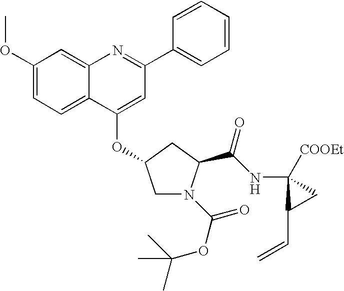

Ethyl-1-[[[(2S,4R)-1-[(1,1-dimethylethoxy)carbonyl]-4-[(7-methoxy-2-phenyl-4-quinolinyl)oxy]-2-pyrrolidinyl]carbonyl]amino]-2-ethenyl-(1R,2S)-cyclopropanecarboxylate: To a solution of (1R,2S)-1-amino-2-vinylcyclopropane carboxylic acid ethyl ester toluenesulfonic acid (2.29 g, 7.0 mmol) and N-Boc (2S, 4R)-(2-phenyl-7-methoxy quinoline-4-oxo)proline (3.4 g, 7.3 mmol) in 100 ml of DCM was added HATU (3.44 g, 9.05 mmol) and then DIEA (3.81 ml, 21.9 mmol) under stirring. The mixture was stirred at r.t. for two hours. After the complete consumption of starting materials, the reaction mixture was washed with brine twice and dried over MgSO₄. After removal of solvent, the crude product was subject to chromatography on silica gel (hexane:EtOAc=2:1). 3.45 g of the title compound was obtained: R_{f} 0.3 (EtOAc:hexane=2:1); MS m/z: 602.36 (M+H⁺).

### Intermediate 8.B-2

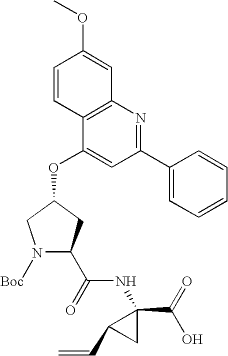

1-[[[(2S,4R)-1-[(1,1-dimethylethoxy)carbonyl]-4-[(7-methoxy-2-phenyl-4-quinolinyl)oxy]-2-pyrrolidinyl]carbonyl]amino]-2-ethenyl-(1R,2S)-cyclopropanecarboxylic acid: To a solution of the product of Intermediate 8.B-1 (1.70 g, 2.83 mmol) in 140 ml of THF/H₂O/MeOH (9:5:1.5) was added lithium hydroxide monohydrate (0.95 g, 22.6 mmol). After stirring at r.t. for 24 hours, the reaction mixture was neutralized with 1.0 N HCl. The organic solvents were evaporated under vacuum, and the remaining aqueous phase was acidified to pH˜3 using 1.0 N HCl and was extracted with EtOAc. The organic layer was washed with brine, and was dried over anhydrous magnesium sulfate. After removal of solvent, 1.6 g of the title compound was obtained: R_{f} 0.2 (EtOAc:MeOH=10:1); MS m/z: 574.36 (M+H⁺).

### Intermediate 8.B-3

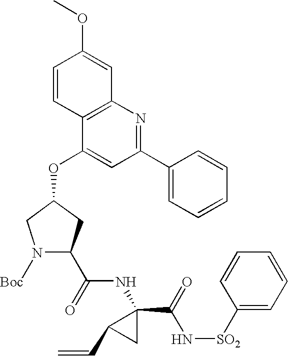

N-(1,1-dimethylethoxy)carbonyl)-(4R)-4-[(7-methoxy-2-phenyl-4-quinolinyl)oxy]-L-prolyl-1-amino-2-ethenyl-N-(phenylsulfonyl)-(1R,2S)-cyclopropanecarboxamide: To a solution of the product of Intermediate 8.B-2 (1.24 g, 2.16 mmol) in 20 ml of DMF was added HATU (0.98 g, 2.58 mmol) and DIEA (1.43 ml, 8.24 mmol), the mixture was stirred for one hour before adding a solution of benzenesulfonamide (1.30 g, 8.24 mmol), DMAP (1.0 g, 8.24 mmol) and DBU (1.29 g, 8.4 mmol) in 15 ml of DMF. Stirring continued for additional four hours. The reaction mixture was diluted with EtOAc and was washed with aqueous NaOAc buffer (pH˜5, 2×10 ml), NaHCO₃ solution and brine. After drying over MgSO₄ and removal of solvent a pure product precipitated by adding one portion of DCM. The filtrate was concentrated and the residue was subjected to chromatography on silica gel using hexane/EtOAc (1:1˜1:2). A total of 0.76 g of the title compound was obtained: R_{f} 0.3 (EtOAc:hexane=3:1), MS m/z: 713.45 (M+H⁺), 735.36 (M+Na⁺).

### Intermediate 8.B-4

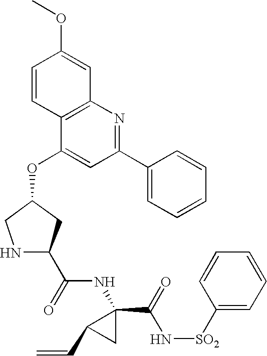

(4R)-4-[(7-methoxy-2-phenyl-4-quinolinyl)oxy]-L-prolyl-1-amino-2-ethenyl-N-(phenylsulfonyl)-(1R,2S)-cyclopropanecarboxamide: To a solution of the product from Intermediate 8.B-3 in 30 ml of DCM was added dropwise 15 ml of TFA. The mixture was stirred at r.t. for two hrs. After removal of solvents, a 20-ml portion of DCM was poured in followed by evaporation to dryness. This process of DCM addition followed by evaporation was repeated four times. Toluene (20 ml) was added and then removed by evaporation to dryness. Two repeats of this cycle gave a residue that solidified into 0.9 g white powder as TFA salt of the title compound. A small sample of the TFA salt was neutralized with NaHCO₃ to obtain the title compound: R_{f} 0.4 (DCM:MeOH=10:1); MS m/z: 613.65 (M+H⁺).

### Intermediate 8.B-5

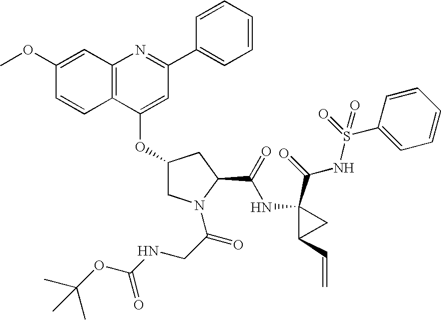

(2S,4R)-1-(2-t-butoxycarbonylaminoacetyl)-4-(7-methoxy-2-phenylquinolin-4-yloxy)-N-((1R,2S)-1-(phenylsulfonylcarbamoyl)-2-vinylcyclopropyl)pyrrolidine-2-carboxamide: To a solution of the product of Intermediate 8.B-4 (0.10 g, 0.15 mmol) and N-Boc-glycine (0.035 g, 0.20 mmol) in 3.0 mL of acetonitrile was added HATU (85.1 mg, 0.22 mmol) and DIEA (0.09 mL, 0.5 mmol) at RT under stirring. The reaction mixture was stirred for 2 h. LC-MS and TLC analysis indicated completion of the coupling reaction. A 20-mL of EtOAc was poured in and the mixture was washed with a buffer (pH˜4, AcONa/AcOH), NaHCO₃ and brine, and was dried over Na₂SO₄. After removal of solvent, the crude product was subject to chromatography on silica gel (eluents: EtOAc/hexane). A total of 0.11 g of the title compound was obtained: R_{f} 0.2 (EtOAc:hexane=2:1); MS m/z: 770.3 (M+H⁺).

### Intermediate 8.B-6

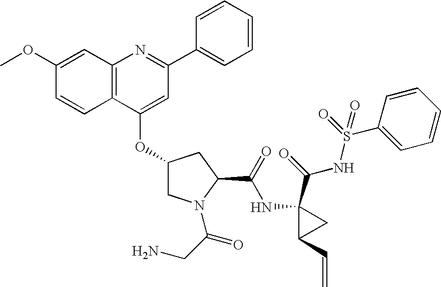

(2S,4R)-1-(2-aminoacetyl)-4-(7-methoxy-2-phenylquinolin-4-yloxy)-N-((1R,2S)-1-(phenylsulfonylcarbamoyl)-2-vinylcyclopropyl)pyrrolidine-2-carboxamide: The product from Intermediate 8.B-5 (0.11 g, 0.13 mmol) was dissolved in 2 mL of 4N HCl in dixoxane and the reaction was stirred for 1 hour at RT. After removal of solvents, a 3-mL portion of DCM was poured in followed by evaporation to dryness. This process of DCM addition followed by evaporation was repeated three times to give the compound Intermediate 6 as its HCl salt (0.10 g). MS m/z: 670.2 (M+H⁺).

### Compound 12 (CNX-221)

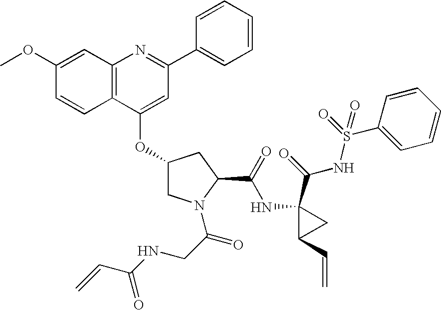

(2S,4R)-1-(2-acrylamidoacetyl)-4-(7-methoxy-2-phenylquinolin-4-yloxy)-N-((1R,2S)-1-(phenylsulfonylcarbamoyl)-2-vinylcyclopropyl)pyrrolidine-2-carboxamide (1-27): The title compound was made by coupling Intermediate 8.B-6 and acrylic acid using HATU following the coupling reactions described for Intermediate 8.B-5. A total of 0.10 g of the title compound was obtained 87%: R_{f} 0.5 (5% MeOH in DCM); MS m/z: 724.3 (M+H⁺).

### Biochemical and Cellular Data

Compound 12 was tested in the replicon assay described in Example 4. Compound 12 had an EC50 of 204 nM in the assay, whereas reversible Compound 11 had an EC50 of greater than 3000 nM in the assay.

### HCV Protease FRET Assay for Wild Type and Mutated NS3/4A 1b Enzymes (IC₅₀)

The protocol is a modified FRET-based assay (v_{—}02) from In Vitro Resistance Studies of HCV Serine Protease Inhibitors, 2004, JBC, vol. 279, No. 17, pp 17508-17514. Inherent potency of compounds was assessed against A156S, A156T, D168A, and D168V mutants of the HCV NS3/4A 1b protease enzyme as follows: 10× stocks of NS3/4A protease enzyme from Bioenza (Mountain View, Calif.) and 1.13×5-FAM/QXL™520 FRET peptide substrate from Anaspec (San Jose, Calif.) were prepared in 50 mM HEPES, pH 7.8, 100 mM NaCl, 5 mM DTT and 20% glycerol. 5 μL of each enzyme were pre-incubated in a Corning (#3573) 384-well, black, non-treated microtiter plate (Corning, N.Y.) for 30 min at 25° C. with a 0.5 μL volume of 50% DMSO and serially diluted compounds prepared in 50% DMSO. Protease reactions were started with the addition of 45 μL of the FRET substrate and monitored for 120 minutes at λₑₓ487/λₑₘ514 through Quad⁴ monochromoters in a Synergy⁴ plate reader from BioTek (Winooski, Vt.). At the conclusion of each assay, progress curves from each well were examined for linear reaction kinetics and fit statistics (R², absolute sum of squares). Initial velocity (0 minutes to 30+ minutes) from each reaction was determined from the slope of a plot of relative fluorescence units vs time (minutes) and then plotted against inhibitor concentration to estimate IC₅₀ from log [Inhibitor] vs Response, Variable Slope model in GraphPad Prism from GraphPad Software (San Diego, Calif.). Results are presented in Table 12.

TABLE 12

| Compound tested | Enzyme/Assay | IC₅₀ (nM) |
|---|---|---|
| Compound 12 | WT | 0.30 |
| | HCV D168A | 17 |
| Reversible Compound 11 | WT | 165 |
| | HCV D168A | Greater than 3000 |

The data show that Compound 12 which contains a warhead that covalently bind HCV protease is a potent inhibitor or wild type and mutant HCV protease, whereas the reversible Compound 11 is not.

The teachings of all patents, published applications and references cited herein are incorporated by reference in their entirety. While this invention has been particularly shown and described with references to example embodiments thereof, it will be understood by those skilled in the art that various changes in form and details may be made therein without departing from the scope of the invention encompassed by the appended claims.

What is claimed is:

1. A method for designing an inhibitor that covalently binds a target polypeptide, comprising:
A) providing a structural model of a reversible inhibitor bound to a binding site in a target polypeptide, wherein the reversible inhibitor makes non-covalent contacts with the binding site;
B) identifying a Cys residue in the binding site of the target polypeptide that is adjacent to the reversible inhibitor when the reversible inhibitor is bound to the binding site;
C) producing structural models of candidate inhibitors that covalently bind the target polypeptide, wherein each candidate inhibitor contains a warhead that is bonded to a substitutable position of the reversible inhibitor, the warhead comprising a reactive chemical functionality and optionally a linker that positions the reactive chemical functionality within bonding distance of the Cys residue in the binding site of the target polypeptide;
D) determining the substitutable positions of the reversible inhibitor that result in the reactive chemical functionality of the warhead being within bonding distance of the Cys residue in the binding site of the target polypeptide when the candidate inhibitor is bound to the binding site;
E) for a candidate inhibitor that contains a warhead that is within bonding distance of the Cys residue in the binding site of the target polypeptide when the candidate inhibitor is bound to the binding site, forming a covalent bond between the sulfur atom of the Cys residue in the binding site and the reactive chemical functionality of the warhead when the candidate inhibitor is bound to the binding site, wherein a covalent bond length of less than about 2 Å indicates that the candidate inhibitor is an inhibitor that covalently binds a target polypeptide.

2. The method of claim 1, wherein the Cys residue is not conserved in the protein family that comprises the target polypeptide.

3. The method of claim 1, wherein the polypeptide has catalytic activity.

4. The method of claim 3, wherein the binding site is a binding site for a substrate or cofactor.

5. The method of claim 3, wherein the Cys residue is not a catalytic residue.

6. The method of claim 1, further comprising:
F) determining whether the binding site is occluded when a covalent bond is formed between the sulfur atom of the Cys residue in the binding site and the reactive chemical functionality of the warhead.

7. The method of claim 1, wherein the covalent bond formed in E) is formed using a computational method in which the warhead and the side chain of the Cys residue are flexible and the remainder of the structures of the candidate inhibitor and the binding site are fixed.

8. The method of claim 1, wherein in B) each Cys residue in the binding site of the target polypeptide that is adjacent to the reversible inhibitor when the reversible inhibitor is bound to the binding site is identified.

9. The method of claim 1, wherein the structural models of candidate inhibitors in C) comprise a plurality of models of candidate inhibitors, wherein the warhead is bonded to a different substitutable position in each member of the plurality.

10. The method of claim 1, wherein the warhead has the formula —X-L-Y, wherein
X is a bond or a bivalent C₁-C₆ saturated or unsaturated, straight or branched hydrocarbon chain wherein optionally one, two or three methylene units of the hydrocarbon chain are independently replaced by —NR—, —O—, —C(O)—, —OC(O)—, —C(O)O—, —S—, —SO—, —SO₂—, —C(═S)—, —C(═NR)—, —N═N—, or —C(═N₂)—;
L is a covalent bond or a bivalent C₁₋₈ saturated or unsaturated, straight or branched, hydrocarbon chain, wherein one, two, or three methylene units of L are optionally and independently replaced by cyclopropylene, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—;
Y is hydrogen, C₁₋₆ aliphatic optionally substituted with oxo, halogen, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with at 1-4 groups independently selected from -Q-Z, oxo, NO₂, halogen, CN, or C₁₋₆ aliphatic, wherein:
Q is a covalent bond or a bivalent C₁₋₆ saturated or unsaturated, straight or branched, hydrocarbon chain, wherein one or two methylene units of Q are optionally and independently replaced by —NR—, —S—, —O—, —C(O)—, —SO—, or —SO₂—; and
Z is hydrogen or C₁₋₆ aliphatic optionally substituted with oxo, halogen, or CN;
each R group is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur
each R group is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur.

11. The method of claim 1, wherein the target polypeptide is a kinase

12. The method of claim 11, wherein the reversible inhibitor interacts with the ATP binding site.

13. The method of claim 12, wherein the reversible inhibitor interacts with the hinge region of the ATP binding site.

14. The method of claim 11, wherein the kinase is a protein kinase.

15. The method of claim 11, wherein the kinase is a lipid kinase.

16. The method of claim 1, wherein the target polypeptide is a protease.

17. The method of claim 16, wherein the protease is a viral protease.

18. The method of claim 17, wherein the viral protease is HCV protease.

19. The method of claim 16, wherein the protease is a caspase.

20. The method of claim 16, wherein the protease is the proteasome or a component of the proteasome.

21. The method of claim 1, wherein the target polypeptide is a phosphodiesterase.

22. The method of claim 1, wherein the target polypeptide is a deacetylase.

23. The method of claim 1, wherein the target polypeptide is a heat shock protein.

24. The method of claim 1, wherein the target polypeptide is a G protein-coupled receptor.

25. The method of claim 1, wherein the target polypeptide is a transferase.

26. The method of claim 1, wherein the target polypeptide is a metalloenzyme.

27. The method of claim 1, wherein the target polypeptide is a nuclear hormone receptor.

28. The method of claim 1, further comprising refining the structure of the compound to tailor reactivity with the —SH group of the Cys residue.

29. The method of claim 1, wherein the structural model of a reversible inhibitor bound to a binding site in a target polypeptide is a three-dimensional structural model.

30. The method of claim 29, wherein the three-dimensional structural model is produced using structural information obtained from a crystal structure or solution structure.

31. The method of claim 30, wherein the three-dimensional structural model is a homology model.

32. The method of claim 30, wherein the three-dimensional structural model is produced using a computational method.

33. The method of claim 1, wherein the method is performed in silico.

34. The method of claim 1, wherein the method is performed using one or more computational methods.

35. The method of claim 1, wherein the polypeptide has catalytic activity and the reversible inhibitor inhibits the activity of the polypeptide with an IC50 of about 50 μM or less.

36. The method of claim 1, wherein the polypeptide has catalytic activity and the reversible inhibitor inhibits the activity of the polypeptide with a Ki of about 50 μM or less.

37. The method of claim 1, wherein the target polypeptide is a mutant or drug-resistant protein.

38. The method of claim 1, wherein the reversible inhibitor is a potent reversible inhibitor.

39. The method of claim 1, wherein the reversible inhibitor inhibits the target polypeptide with weak or moderate potency.

40. The method of claim 39, wherein the inhibitor that covalently binds a target polypeptide inhibits the target polypeptide with improved potency relative to the reversible inhibitor.

41. A method for designing an inhibitor that covalently binds a target polypeptide, comprising:
A) providing a structural model of a reversible inhibitor bound to a binding site in a target polypeptide, wherein the reversible inhibitor makes non-covalent contacts with the binding site;
B) identifying a Cys residue in the binding site of the target polypeptide that is adjacent to the reversible inhibitor when the reversible inhibitor is bound to the binding site;
C) providing a structural model of a warhead group, the warhead comprising a reactive chemical functionality capable of reacting with the Cys residue and forming a covalent bond between the sulfur atom of the Cys residue in the binding site and the reactive chemical functionality of the warhead group;
D) identifying a substitutable position of the reversible inhibitor to which the warhead group can be bonded, optionally through a linker, such that the bond formed between the sulfur atom of the Cys residue in the binding site and the reactive chemical functionality of the warhead group has a bond length of less than about 2 Å;
E) bonding the warhead group, optionally through the linker, to the substitutable position of the reversible inhibitor.

42. An irreversible inhibitor comprising a chemical moiety that binds to a binding site on a target polypeptide and a warhead containing a conjugated enone.

43. The irreversible inhibitor of claim 42, wherein the warhead has the formula 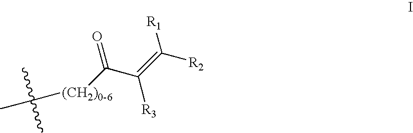
wherein R₁, R₂ and R₃ are independently hydrogen, C₁-C₆ alkyl, or C₁-C₆ alkyl that is substituted with —NRxRy; and
Rx and Ry are independently hydrogen or C₁-C₆ alkyl.

44. A polypeptide conjugate, wherein the conjugate is the reaction product of an irreversible inhibitor that contains a conjugated enone warhead and a polypeptide that comprises a cysteine, and has the formula
X-M-S—CH₂—R
wherein:
X is a chemical moiety that binds to the binding site of a target polypeptide, wherein the binding site of the target polypeptide contains a cysteine residue;
M is a modifier moiety formed by the covalent bonding of an enone-containing warhead with the sulfur atom of said cysteine residue;
S—CH₂ is the side chain sulfur-methylene of said cysteine residue; and
R is the remainder of the target polypeptide.

45. The polypeptide conjugate of claim 44, wherein the conjugate is of the formula: 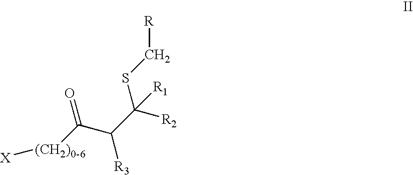
wherein X is a chemical moiety that binds to the binding site of a target polypeptide, wherein the binding site contains a cysteine residue;
S—CH₂ is the side chain of said cysteine residue;
R is the remainder of the target polypeptide;
R₁, R₂ and R₃ are independently hydrogen, C₁-C₆ alkyl, or C₁-C₆ alkyl that is substituted with —NRxRy; and
Rx and Ry are independently hydrogen or C₁-C₆ alkyl.
